(19) United States
(12) Patent Application Publication
(10) Pub. No.: US 2026/0015372 A1
(43) Pub. Date: Jan. 15, 2026

(54) Organophosphorous Compounds and Uses Thereof

(71) Applicant: JAL Therapeutics, LLC, Irvine, CA (US)

(72) Inventors: Kensaku Nakayama, Irvine, CA (US); Mark Maricich, Irvine, CA (US); David Maricich, Irvine, CA (US)

(21) Appl. No.: 18/994,533

(22) PCT Filed: Jul. 20, 2023
(86) PCT No.: PCT/US2023/070627

§ 371 Date: Jan. 14, 2025

Related U.S. Application Data
(60) Provisional application No. 63/390,937, filed on Jul. 20, 2022.

(51) Int. Cl. C07F 9/40; A61K 31/662; A61P 25/28

(52) U.S. Cl. CPC C07F 9/4056; A61K 31/662; A61P 25/28

(57) ABSTRACT

Provided herein are organophosphorous compounds useful as inhibitors of esterase proteins and in treating esterase-related diseases.

### RELATED APPLICATIONS

This application claims the benefit of Patent Cooperation Treaty Application No. PCT/US2023/070627, titled “ORGANOPHOSPHOROUS COMPOUNDS AND USES THEREOF”, filed Jul. 20, 2023, and U.S. Provisional patent application 63/390,937 filed Jul. 20, 2022, the entire contents of which are incorporated by reference herein.

### FIELD

The present disclosure relates to organophosphorous compounds, and their uses including for the treatment of diseases.

### BACKGROUND

Esterases, including cholinesterases, are involved in a variety of roles in the body. Cholinesterases include Butyrylcholinesterase (BCHE) and acetylcholinesterase (ACHE), among others. Cholinesterases act on choline-based esters, some of which include neurotransmitters, such as acetylcholine, which when released from a motor nerve terminal diffuses through the synaptic cleft, binds nicotinic receptors on the motor end plate and results in muscle contraction. ACHE, for example, lyses acetylcholine and results in muscle relaxation. Because of their essential function, chemicals that interfere with the action of cholinesterases are neurotoxins. Therefore, care must be taken when using cholinesterase inhibitors to utilize effective, yet non-neurotoxic, amounts in a therapeutic context. Furthermore, therapeutic use of pan-esterase inhibitors (e.g., non-specific esterase inhibitors) may result in adverse peripheral effects. For example, currently utilized cholinesterase inhibitors are non-specific and present such adverse peripheral effects as a result of their ACHE inhibition. Progression of some diseases may occur concomitantly with differential changes in cholinesterase activity, e.g., ACHE activity decreases while BCHE activity increases.

In view of the role that esterases play in many disease states, including those in the nervous system, there is an urgent and continuing need for small molecule ligands which inhibit or modulate the activity of esterases, and that do so in a selective or targeted manner.

### SUMMARY

Described herein is the compound di-n-butyl 2-naphthylmethylphosphonate (dibutyl (naphthalen-2-ylmethyl)phosphonate),

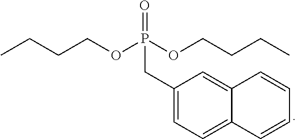 [SMILES: CCCCOP(=O)(Cc1ccc2ccccc2c1)OCCCC]

Also described herein is the compound di-n-butyl 3,5-dimethylbenzylphosphonate (dibutyl (3,5-dimethylbenzyl)phosphonate),

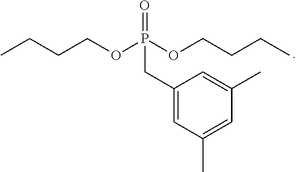 [SMILES: CCCCOP(=O)(Cc1cc(C)cc(C)c1)OCCCC]

Also described herein is the compound di-n-butyl 2 chlorophenyl phosphate,

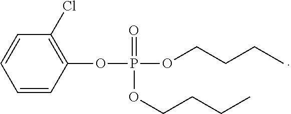 [SMILES: CCCCOP(=O)(OCCCC)Oc1ccccc1Cl]

Also described herein are uses of dibutyl (naphthalen-2-ylmethyl)phosphonate, dibutyl (3,5-dimethylbenzyl)phosphonate or di-n-butyl 2 chlorophenyl phosphate, as well as other organophosphorous compounds, including inhibition of esterases and treatment of related diseases.

In some embodiments, provided herein are compositions, comprising dibutyl (naphthalen-2-ylmethyl)phosphonate, dibutyl (3,5-dimethylbenzyl)phosphonate or di-n-butyl 2 chlorophenyl phosphate.

In some embodiments, provided herein are pharmaceutical compositions comprising dibutyl (naphthalen-2-ylmethyl)phosphonate, dibutyl (3,5-dimethylbenzyl)phosphonate or di-n-butyl 2 chlorophenyl phosphate, and a pharmaceutically acceptable carrier.

In some embodiments, provided herein are methods of treating an esterase-related disease, e.g., a cholinesterase-related disease, e.g., an ACHE-related or BCHE-related disease, in a subject in need thereof, comprising administering to the subject a therapeutically effective amount of dibutyl (naphthalen-2-ylmethyl)phosphonate, dibutyl (naphthalen-2-ylmethyl)phosphonate in a composition or pharmaceutical composition, dibutyl (3,5-dimethylbenzyl)phosphonate, dibutyl (3,5-dimethylbenzyl)phosphonate in a composition or pharmaceutical composition, or di-n-butyl 2 chlorophenyl phosphate in a composition or pharmaceutical composition.

In some embodiments, the esterase-related disease includes a disease associate with the cholinergic anti-inflammatory pathway.

In some embodiments, the esterase-related disease includes an autoimmune disease.

In some embodiments, the esterase-related disease includes an inflammatory disease.

In some embodiments, the esterase-related disease includes a disease associated with cholinergic dysfunction or acetylcholine (ACH) deficiency.

In some embodiments, the esterase-related disease includes, but is not limited to, long COVID disease, or symptoms thereof, which may be associated with inflammation, cholinergic dysfunction, or formation of beta-amyloid plaques, or a combination thereof.

In some embodiments, the long COVID disease includes inflammatory response, cholinergic dysfunction, or Alzheimer's-like symptoms.

In some embodiments, provided herein are methods of treating a disease in a subject in need thereof, comprising administering a therapeutically effective amount of dibutyl (naphthalen-2-ylmethyl)phosphonate, dibutyl (naphthalen-2-ylmethyl)phosphonate in a composition or pharmaceutical composition, dibutyl (3,5-dimethylbenzyl)phosphonate, dibutyl (3,5-dimethylbenzyl)phosphonate in a composition or pharmaceutical composition, or di-n-butyl 2 chlorophenyl phosphate in a composition or pharmaceutical composition to the subject, wherein the disease is selected from inflammatory diseases, diseases associated with cholinergic dysfunction, autoimmune diseases, neurodegenerative disorders including Alzheimer's disease (AD), mild cognitive impairment (MCI), dementia with Lewy Bodies, subcortical vascular dementia, Parkinson's disease (PD), Huntington's disease (HD), and amyotrophic lateral sclerosis (ALS), or a combination thereof.

In some embodiments, provided herein are methods of treating a disease in a subject in need thereof, comprising administering a therapeutically effective amount of dibutyl (naphthalen-2-ylmethyl)phosphonate, dibutyl (naphthalen-2-ylmethyl)phosphonate in a composition or pharmaceutical composition, dibutyl (3,5-dimethylbenzyl)phosphonate, dibutyl (3,5-dimethylbenzyl)phosphonate in a composition or pharmaceutical composition, or di-n-butyl 2 chlorophenyl phosphate in a composition or pharmaceutical composition to the subject, wherein the disease is selected from an inflammatory disease including, type 2 diabetes mellitus, hypertension, insulin resistance, hyperlipidemia, obesity, heart disease, metabolic syndrome, hyperthyroidism, nephrotic syndrome/kidney disease, proliferative diabetic retinopathy, age-related macular degeneration, chronic alcoholism, a viral infection, or a combination thereof.

In some embodiments, the inflammatory disease includes type 2 diabetes mellitus, metabolic syndrome, an ophthalmic disease, or a viral infection, or a combination thereof.

In some embodiments, provided herein are methods of treating a disease associated with cholinergic dysfunction, comprising administering a therapeutically effective amount of dibutyl (naphthalen-2-ylmethyl)phosphonate, dibutyl (naphthalen-2-ylmethyl)phosphonate in a composition or pharmaceutical composition, dibutyl (3,5-dimethylbenzyl)phosphonate, dibutyl (3,5-dimethylbenzyl)phosphonate in a composition or pharmaceutical composition, or di-n-butyl 2 chlorophenyl phosphate in a composition or pharmaceutical composition to the subject.

In some embodiments, the disease associated with cholinergic dysfunction is selected from Alzheimer's disease, dementia with Lewy Bodies, mild cognitive impairment (MCI), myasthenia gravis, multiple sclerosis (MS), autism spectrum disorder (ASD), Parkinson's disease, Huntington's disease, Lambert-Eaton myasthenic syndrome (LEMS), subcortical vascular dementia, opioid addiction, opiate use disorder, bipolar disorder, schizophrenia, metabolic syndrome, or glaucoma, among other cholinergic disorders.

In some embodiments, the disease associated with cholinergic dysfunction includes mild cognitive impairment (MCI), dementia with Lewy Bodies, myasthenia gravis, multiple sclerosis (MS), autism spectrum disorder (ASD), opioid addiction, bipolar disorder, schizophrenia, an autoimmune disorder, or a combination thereof.

In some embodiments, the subject includes a genetic predisposition to increased risk of having AD or increased prevalence of beta-amyloid peptides or formation of beta-amyloid plaques.

In some embodiments, the genetic predisposition includes APOE e4 Gene, other Late-Onset Alzheimer's Genes (ABCA7, CLU, CR1, PICALM, PLD3, TREM2, or SORL1, or a combination thereof), Young-Onset Alzheimer's, Family History and Genetics Mutations, genetic mutations of amyloid precursor protein (APP), Presenilin 1 (PSEN1), Presenilin 2 (PSEN2), and Down Syndrome Mutations.

In some embodiments, the genetic predisposition includes APOE e4 gene, Young-Onset Alzheimer's, or Down Syndrome, or a combination thereof.

In some embodiments, provided herein are methods of modulating differentiation of a stem cell, comprising contacting the stem cell with an effective amount of dibutyl (naphthalen-2-ylmethyl)phosphonate, dibutyl (naphthalen-2-ylmethyl)phosphonate in a composition or pharmaceutical composition, dibutyl (3,5-dimethylbenzyl)phosphonate, dibutyl (3,5-dimethylbenzyl)phosphonate in a composition or pharmaceutical composition, or di-n-butyl 2 chlorophenyl phosphate in a composition or pharmaceutical composition.

In some embodiments, provided herein are methods of modulating (e.g., inhibiting or reducing) expression or production of amyloid-β precursor protein (APP) in a subject in need thereof, comprising administering a therapeutically effective amount of dibutyl (naphthalen-2-ylmethyl)phosphonate, dibutyl (naphthalen-2-ylmethyl)phosphonate in a composition or pharmaceutical composition, dibutyl (3,5-dimethylbenzyl)phosphonate, dibutyl (3,5-dimethylbenzyl)phosphonate in a composition or pharmaceutical composition, or di-n-butyl 2 chlorophenyl phosphate in a composition or pharmaceutical composition to the subject.

In some embodiments, provided herein are methods of modulating (e.g., inhibiting or reducing) expression or production of intracellular neurofibrillary tangles made of hyperphosphorylated tau protein in a subject in need thereof, comprising administering a therapeutically effective amount of dibutyl (naphthalen-2-ylmethyl)phosphonate, dibutyl (naphthalen-2-ylmethyl)phosphonate in a composition or pharmaceutical composition, dibutyl (3,5-dimethylbenzyl)phosphonate, dibutyl (3,5-dimethylbenzyl)phosphonate in a composition or pharmaceutical composition, or di-n-butyl 2 chlorophenyl phosphate in a composition or pharmaceutical composition to the subject.

In some embodiments, CNS/PNS disorders associated with acetylcholine (ACH) deficiency may benefit from the treatment of highly specific BCHE inhibition obtained from the compounds and compositions discussed above and below. ACh deficiency or dysregulation of the cholinergic system may be addressed using highly selective BCHE inhibitors (see e.g., Compounds in Table 1), which are designed to reduce the hydrolysis of ACh (e.g., decreasing the hydrolysis of ACH from elevated levels of BCHE in order to increase ACH neurotransmitter levels) and thus increase its availability as a neurotransmitter. These inhibitors hold potential for the treatment of various diseases associated with ACh deficiency. By reducing the breakdown of Ach, these compounds increase its availability as a neurotransmitter, potentially improving cognitive function, motor control, and ameliorating symptoms in neurodegenerative and behavioral disorders. Furthermore, they may provide benefits in peripheral somatic disorders and peripheral autonomic dysfunctions by compensating for ACh imbalances, including the following.

### Central Nervous System (CNS)/Neurodegenerative Disorders

In the central nervous system (CNS), where ACh is crucial for cognitive functions, these highly selective BCHE inhibitors may be used to alleviate symptoms in neurodegenerative disorders such as Alzheimer's disease, Parkinson's disease, Lewy body dementia, vascular dementia, mild cognitive impairment (MCI), traumatic brain injury, amyotrophic lateral sclerosis (ALS), multiple sclerosis and Huntington's disease. By inhibiting the breakdown of ACh, these compounds may be designed to enhance cholinergic neurotransmission, leading to improved cognitive function, memory, and motor control.

### Central Nervous System (CNS)/Behavioral Disorders

BCHE inhibitors also have implications for behavioral disorders such as schizophrenia, autism, opioid addiction, and bipolar disorder. By increasing ACh levels, compounds may be designed to help modulate cognitive processes, emotional regulation, reward pathways, and social behavior, potentially reducing the severity of symptoms associated with these conditions.

### Peripheral Nervous System (PNS)/Peripheral Somatic Disorders

In addition to their effects in the CNS, these highly selective BCHE inhibitors may impact the peripheral nervous system (PNS) as well. By enhancing ACh availability, these compounds may be designed to be beneficial in peripheral somatic disorders like myasthenia gravis, where ACh receptor function is impaired. By reducing the breakdown of ACh, these compounds may be developed to help compensate for receptor dysfunction, leading to improved muscle strength and reduced fatigue.

### Peripheral Nervous System (PNS)/Peripheral Autonomic Disorders

Moreover, BCHE inhibitors may have a positive impact on peripheral autonomic disorders, including those related to sympathetic and parasympathetic responses. By increasing ACh levels, these inhibitors can influence physiological processes such as insulin secretion, glucose regulation, lipid metabolism, and immune responses. Therefore, they hold potential for treating conditions like type 2 diabetes mellitus, metabolic syndrome, kidney disease, glaucoma, chronic inflammation, postural orthostatic tachycardia syndrome (POTS) and autoimmune diseases.

In some embodiments, specific CNS diseases may be treated with cholinesterase inhibitors that are associated with elevated BCHE.

### Alzheimer's Disease

Current cholinesterase inhibitors, such as donepezil, rivastigmine, and galantamine, are frequently prescribed to manage symptoms and slow the progression of Alzheimer's disease. These medications increase the levels of ACh in the brain, compensating for the cholinergic deficits seen in Alzheimer's. However, these drugs inhibit acetylcholinesterase (ACHE) are also known for severe side effects, in contrast with the compounds of the present disclosure, which may be developed solely as highly selective BCHE inhibitors, and may be inactive towards ACHE, and may be designed to have less side effects.

### Parkinson's Disease

Cholinesterase inhibitors may be used in the treatment of cognitive impairment associated with Parkinson's disease. They may help alleviate cognitive symptoms and improve overall cognitive function.

### Lewy Body Dementia

Lewy body dementia is another neurodegenerative disorder in which cholinesterase inhibitors, particularly donepezil, are often used to manage cognitive symptoms and may improve memory, attention, and other cognitive functions.

### Vascular Dementia

Cholinesterase inhibitors, such as donepezil, rivastigmine, and galantamine, may be prescribed to manage cognitive symptoms in individuals with vascular dementia. These medications may help improve memory, attention, and other cognitive functions.

### Mild Cognitive Impairment (MCI)

Cholinesterase inhibitors, such as donepezil, may be used in the treatment of mild cognitive impairment, which is a condition characterized by cognitive decline that may not be severe enough to meet the criteria for dementia. These medications may help delay or prevent further cognitive decline.

### Traumatic Brain Injury

Cholinesterase inhibitors, such as donepezil, may be used to address cognitive impairment in individuals who have experienced traumatic brain injury. These medications may help improve cognitive function and memory.

### Amyotrophic Lateral Sclerosis (ALS)

Cholinesterase inhibitors, such as rivastigmine, may be used to manage cognitive symptoms associated with ALS. These medications may help improve cognitive function and attention.

In some embodiments, specific PNS diseases may be treated with cholinesterase inhibitors that are associated with elevated BCHE.

### Myasthenia Gravis

Myasthenia gravis is an autoimmune neuromuscular disorder (peripheral somatic disorder) characterized by muscle weakness and fatigue. Cholinesterase inhibitors, such as pyridostigmine, are commonly used to enhance neuromuscular transmission and may improve muscle strength in affected individuals.

In some embodiments, the effects that the compounds of this disclosure have on tau and/or inflammation may be examined.

In some embodiments, provided herein are methods of treating an esterase-related disease, e.g., a cholinesterase-related disease, e.g., an ACHE-related or BCHE-related disease, in a subject in need thereof, comprising administering to the subject a therapeutically effective amount of a compound, or a pharmaceutical composition comprising the compound, and a pharmaceutically acceptable carrier, wherein the compound is selected from:
A) Formula 1:

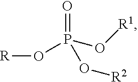

or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2:

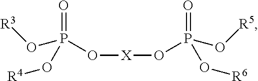

or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A:

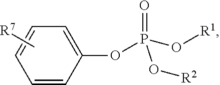

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B:

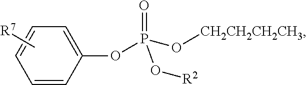

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C:

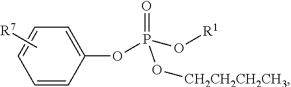

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D:

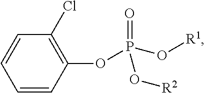

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A:

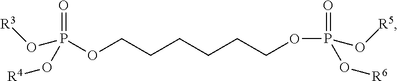

or a pharmaceutically acceptable salt thereof, wherein, R¹ is optionally substituted C₁₋₁₂ alkyl; R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B:

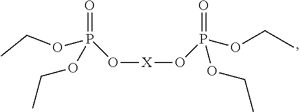

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C:

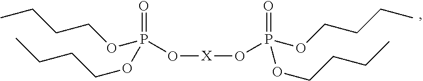

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D:

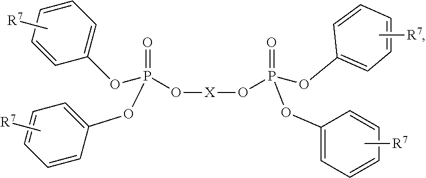

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3:

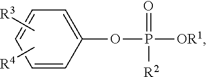

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl; R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
L) Formula 4:

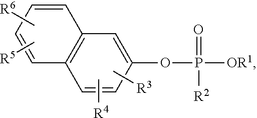

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5:

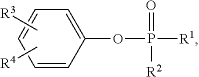

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or
R⁶ is H, C₁₋₆ alkyl, O—(C₁₋₆ alkyl), or an electron withdrawing group (e.g., halo, CN, or NO₂); or
N) Formula 6:

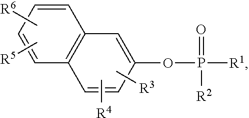

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7:

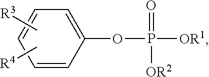

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8:

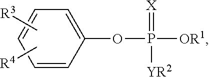

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
Q) Formula 9:

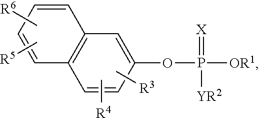

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂),
wherein the esterase-related disease includes:
   a disease associate with the cholinergic anti-inflammatory pathway;
   an autoimmune disease;
   an inflammatory disease;
   a disease associated with cholinergic dysfunction; or
   long COVID disease, or symptoms thereof, which may be associated with inflammation, cholinergic dysfunction, or formation of beta-amyloid plaques, or a combination thereof.

In some embodiments, the long COVID disease includes inflammatory response, cholinergic dysfunction, or Alzheimer's-like symptoms.

In some embodiments, provided herein are methods of treating a disease in a subject in need thereof, comprising administering a therapeutically effective amount of a compound, or a pharmaceutical composition comprising the compound, and a pharmaceutically acceptable carrier, wherein the compound is selected from:
A) Formula 1:

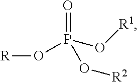

or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2:

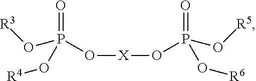

or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A:

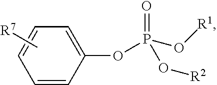

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B:

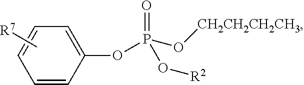

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C:

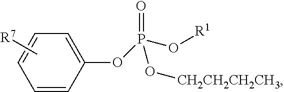

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D:

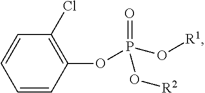

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A:

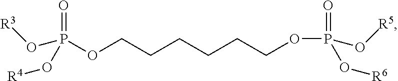

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B:

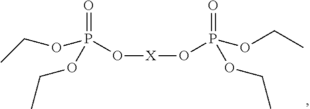

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C:

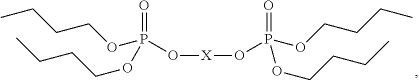

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D:

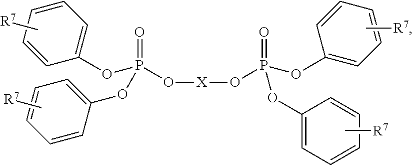

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4
heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3:

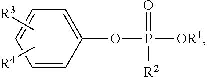

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
L) Formula 4:

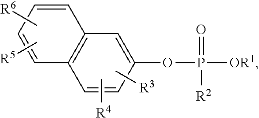

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5:

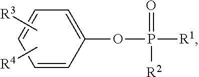

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
N) Formula 6:

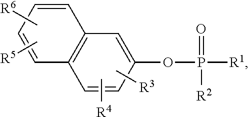

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7:

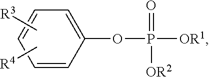

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8:

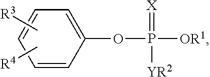

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
Q) Formula 9:

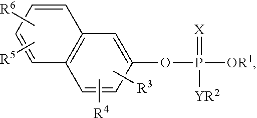

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂),
   wherein the disease is selected from inflammatory diseases, diseases associated with cholinergic dysfunction, autoimmune diseases, neurodegenerative disorders including Parkinson's disease (PD), Huntington's disease (HD), and amyotrophic lateral sclerosis (ALS), or a combination thereof.

In some embodiments, provided herein are methods of treating a disease in a subject in need thereof, comprising administering a therapeutically effective amount of a compound, or a pharmaceutical composition comprising the compound, and a pharmaceutically acceptable carrier, wherein the compound is selected from:
A) Formula 1:

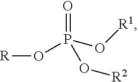

or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2:

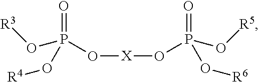

or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A:

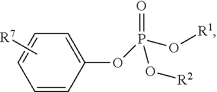

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B:

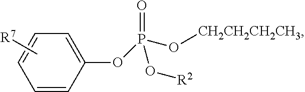

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C:

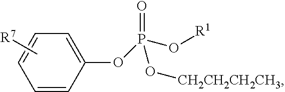

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D:

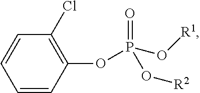

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A:

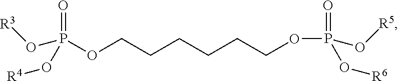

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B:

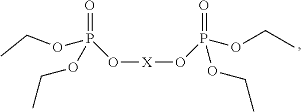

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C:

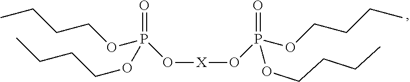

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D:

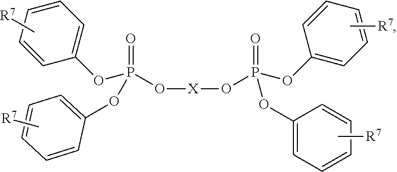

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3:

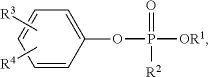

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or
R⁶ is H, C₁₋₆ alkyl, O—(C₁₋₆ alkyl), or an electron withdrawing group (e.g., halo, CN, or NO₂); or
L) Formula 4:

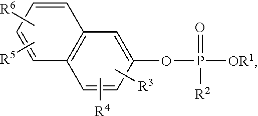

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5:

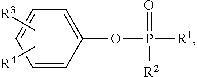

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
N) Formula 6:

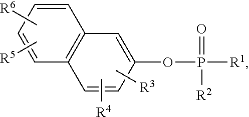

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7:

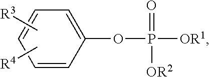

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8:

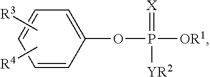

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
Q) Formula 9:

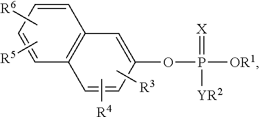

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂),
   wherein the disease is selected from an inflammatory disease including, type 2 diabetes mellitus, hypertension, insulin resistance, hyperlipidemia, obesity, heart disease, metabolic syndrome, hyperthyroidism, nephrotic syndrome/kidney disease, proliferative diabetic retinopathy, age-related macular degeneration, chronic alcoholism, a viral infection, or a combination thereof.

In some embodiments, the inflammatory disease includes type 2 diabetes mellitus, metabolic syndrome, an ophthalmic disease, or a viral infection, or a combination thereof.

In some embodiments, provided herein are methods of treating a disease associated with cholinergic dysfunction in a subject in need thereof, comprising administering to the subject a therapeutically effective amount of a compound, or a pharmaceutical composition comprising the compound, and a pharmaceutically acceptable carrier, wherein the compound is selected from:
A) Formula 1:

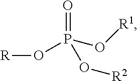

or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2:

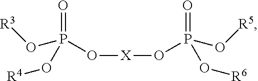

or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A:

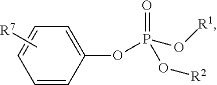

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B:

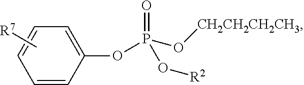

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C:

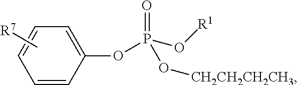

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D:

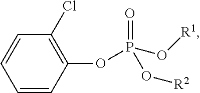

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A:

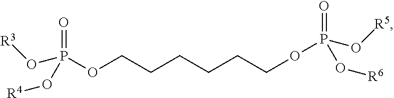

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B:

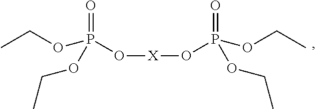

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C:

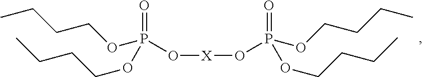

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D:

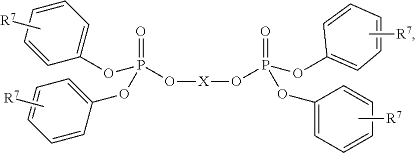

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3:

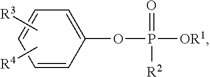

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
L) Formula 4:

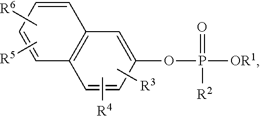

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5:

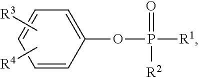

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or
R⁶ is H, C₁₋₆ alkyl, O—(C₁₋₆ alkyl), or an electron withdrawing group (e.g., halo, CN, or NO₂); or
N) Formula 6:

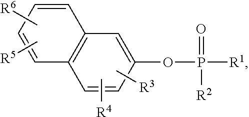

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7:

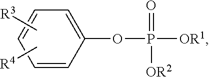

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8:

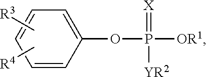

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
Q) Formula 9:

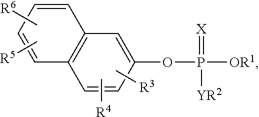

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂).

In some embodiments, the disease associated with cholinergic dysfunction is selected from dementia with Lewy Bodies, myasthenia gravis, multiple sclerosis (MS), autism spectrum disorder (ASD), Parkinson's disease, Huntington's disease, Lambert-Eaton myasthenic syndrome (LEMS), subcortical vascular dementia, opioid addiction, opiate use disorder, bipolar disorder, schizophrenia, metabolic syndrome, or glaucoma, among other cholinergic disorders.

In some embodiments, the disease associated with cholinergic dysfunction includes dementia with Lewy Bodies, myasthenia gravis, multiple sclerosis (MS), autism spectrum disorder (ASD), opioid addiction, bipolar disorder, schizophrenia, an autoimmune disorder, or a combination thereof.

In some embodiments, the subject includes a genetic predisposition to increased risk of having AD or increased prevalence of beta-amyloid peptides or formation of beta-amyloid plaques.

In some embodiments, the genetic predisposition includes APOE e4 Gene, other Late-Onset Alzheimer's Genes (ABCA7, CLU, CR1, PICALM, PLD3, TREM2, or SORL1, or a combination thereof), Young-Onset Alzheimer's, Family History and Genetics Mutations, genetic mutations of amyloid precursor protein (APP), Presenilin 1 (PSEN1), Presenilin 2 (PSEN2), and Down Syndrome Mutations.

In some embodiments, the genetic predisposition includes APOE e4 gene, Young-Onset Alzheimer's, or Down Syndrome, or a combination thereof.

In some embodiments, provided herein are methods of modulating (e.g., inhibiting or reducing) expression or production of amyloid-β precursor protein (APP) in a subject in need thereof, comprising administering to the subject a therapeutically effective amount of a compound, or a pharmaceutical composition comprising the compound, and a pharmaceutically acceptable carrier, wherein the compound is selected from:
A) Formula 1:

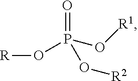

or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2:

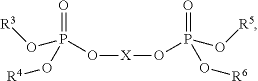

or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A:

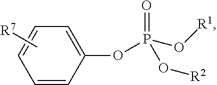

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B:

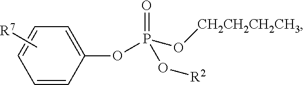

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C:

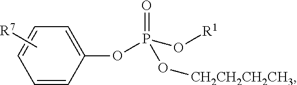

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D:

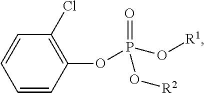

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A:

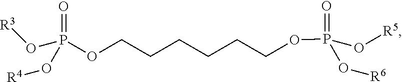

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B:

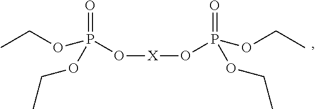

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C:

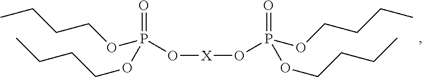

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D:

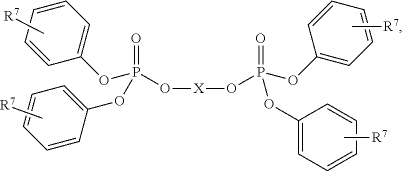

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3:

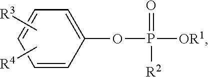

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
L) Formula 4:

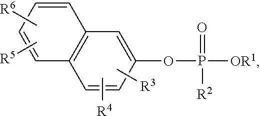

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5:

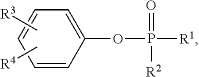

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or
R⁶ is H, C₁₋₆ alkyl, O—(C₁₋₆ alkyl), or an electron withdrawing group (e.g., halo, CN, or NO₂); or
N) Formula 6:

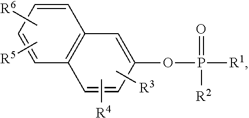

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7:

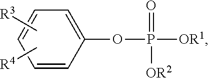

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8:

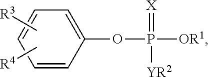

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
Q) Formula 9:

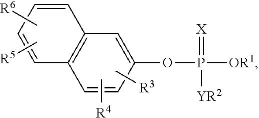

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂).

In some embodiments, provided herein are methods of modulating (e.g., inhibiting or reducing) expression or production of intracellular neurofibrillary tangles made of hyperphosphorylated tau protein in a subject in need thereof, comprising administering to the subject a therapeutically effective amount of a compound, or a pharmaceutical composition comprising the compound, and a pharmaceutically acceptable carrier, wherein the compound is selected from:
A) Formula 1:

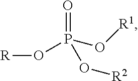

or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2:

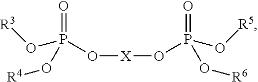

or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A:

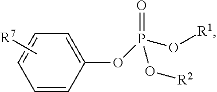

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B:

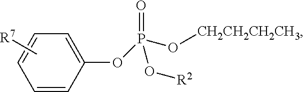

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C:

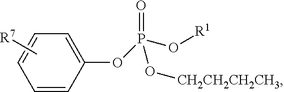

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D:

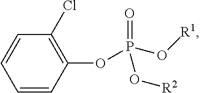

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A:

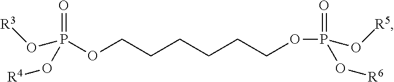

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B:

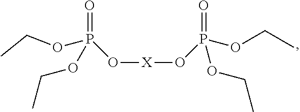

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C:

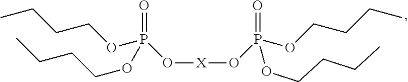

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D:

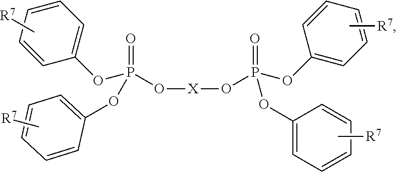

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4
heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3:

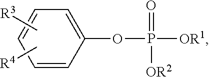

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or
R⁶ is H, C₁₋₆ alkyl, O—(C₁₋₆ alkyl), or an electron withdrawing group (e.g., halo, CN, or NO₂); or
L) Formula 4:

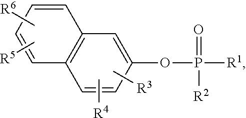

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5:

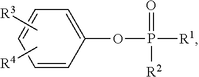

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
N) Formula 6:

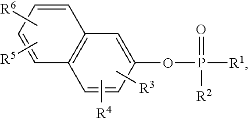

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7:

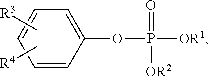

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8:

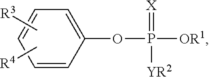

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
Q) Formula 9:

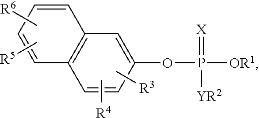

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂).

In some embodiments, provided herein are methods of modulating differentiation of a stem cell, comprising contacting the stem cell with an effective amount of a compound, or a pharmaceutical composition comprising the compound, and a pharmaceutically acceptable carrier, wherein the compound is selected from:
A) Formula 1:

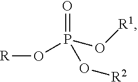

or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2:

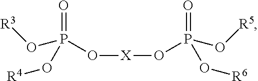

or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A:

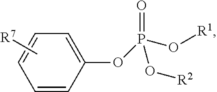

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B:

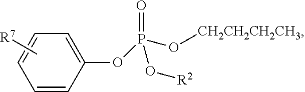

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C:

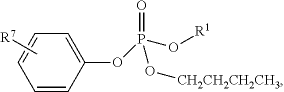

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D:

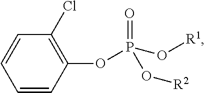

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A:

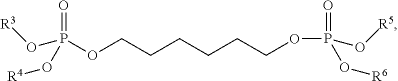

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B:

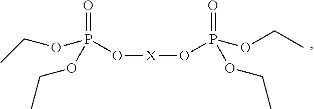

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C:

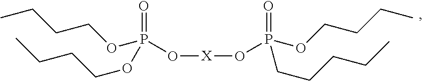

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D:

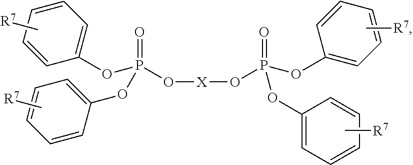

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3:

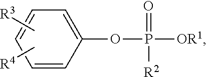

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
L) Formula 4:

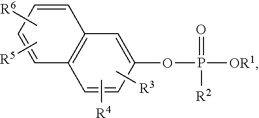

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5:

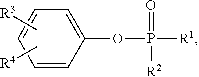

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
N) Formula 6:

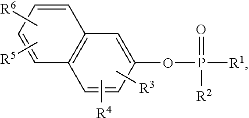

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7:

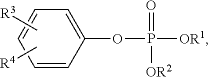

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8:

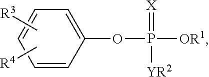

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
Q) Formula 9:

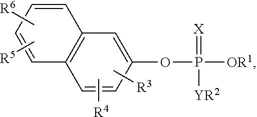

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂).

In some embodiments, the compound is:
2-Bromophenyl di-n-butyl phosphate;
3-Bromophenyl n-dibutyl phosphate;
Dibutyl 3-chlorophenyl phosphate;
4-Bromophenyl di-n-butyl phosphate;
2-Chlorophenyl dimethyl phosphate;
2-Chlorophenyl diethyl phosphate;
2-Chlorophenyl di-n-propyl phosphate;
2-Chlorophenyl di-i-propyl phosphate;
Di-n-butyl 2-chlorophenyl phosphate;
Di-i-butyl 2-Chlorophenyl phosphate;
2-Chlorophenyl di-n-pentyl phosphate;
2-Chlorophenyl dicyclohexyl phosphate;
Di-n-butyl 4-chlorophenyl phosphate;
Di-n-butyl 2,4-dichlorophenyl phosphate;
Di-n-butyl 2-fluorophenyl phosphate;
Di-n-butyl 3-fluorophenyl phosphate;
Dibutyl 2-chlorophenyl thiophosphate;
n-Butyl bis(2,4-dichlorophenyl) phosphate;
Butyl bis(2-chlorophenyl) phosphate;
2-Chlorophenyl diphenyl phosphate;
Di-n-butyl 4-nitrophenyl phosphate;
Di-n-butyl 3-nitrophenyl phosphate;
Di-n-butyl 2-methylphenyl phosphate;
Di-n-butyl 3-methylphenyl phosphate;
Di-n-butyl 4-methylphenyl phosphate;
Di-n-butyl 3,4-dimethylphenyl phosphate;
Di-n-butyl 3,5-dimethylphenyl phosphate;
Diethyl 3,5-dimethylphenyl phosphate;
Di-n-butyl 3,4,5-trimethylphenyl phosphate;
Benzyl di-n-butyl phosphate;
Di-n-butyl 2-methylbenzyl phosphate;
Di-n-butyl 3-methylbenzyl phosphate;
Di-n-butyl 4-methylbenzyl phosphate;
Di-n-butyl 1-napthyl phosphate;
Di-n-butyl 2-napthyl phosphate;
Diethyl 2-napthyl phosphate;
Di-n-butyl 2-phenylethyl phosphate;
Butyl 2-chlorophenyl hexylphosphonate;
Bis(2-chlorophenyl) ethylphosphonate;
Butyl 2-chlorophenyl ethylphosphonate;
Bis(3-nitrophenyl) ethylphosphonate;
Butyl p-nitrophenyl ethylphosphonate;
Bis (p-nitrophenyl) ethylphosphonate;
Bis(2-chlorophenyl) hexylphosphonate;
Bis(2-chlorophenyl) n-butylphosphonate;
Bis(2-chlorophenyl) ethylphosphonate;
2-Chlorophenyl di-n-pentylphosphinate;
2-Chlorophenyl di-n-butylphosphinate;
Phenyl di-n-pentylphosphinate;
4-nitrophenyl dipentylphosphinate;
O-n-butyl-O-2-chlorophenyl-N-hexyl phosphoramidate;
Bis(2-chlorophenyl) N-hexyl phosphoramidate;
Tetraethyl propane-1,3-diyl bisphosphate;
Tetraethyl butane-1,4-diyl bisphosphate;
Tetraethyl pentane-1,5-diyl bisphosphate;
Tetraethyl hexane-1,6-diyl bisphosphate;
Tetraethyl heptane-1,7-diyl bisphosphate;
Tetraethyl 3-oxapentane-1,5-diyl bisphosphate;
Tetrabutyl propane-1,3-diyl bisphosphate;
Tetrabutyl butane-1,4-diyl bisphosphate;
Tetrabutyl pentane-1,5-diyl bisphosphate;
Tetrabutyl hexane-1,6-diyl bisphosphate;
Tetrabutyl heptane-1,7-diyl bisphosphate;
Tetrabutyl octane-1,8-diyl bisphosphate;
Tetrabutyl 3-oxapentane-1,5-diyl bisphosphate;
Tetrabutyl 3-hexene-1,6-diyl bisphosphate;
Tetraphenyl hexane-1,6-diyl bisphosphate;
O,O,O′,O′-tetrabutyl S,S′-(ethane-1,2-diyl) Bis(phosphorothioate);
O,O,O′,O′-tetrabutyl S,S′-(propane-1,3-diyl) Bis(phosphorothioate);
O,O,O′,O′-tetrabutyl S,S′-(butane-1,4-diyl) Bis(phosphorothioate);
O,O,O′,O′-tetrabutyl S,S′-(pentane-1,5-diyl) Bis(phosphorothioate);
O,O,O′,O′-tetrabutyl S,S′-(hexane-1,6-diyl) Bis(phosphorothioate);
O,O,O′,O′-tetrabutyl S,S′-(heptane-1,7-diyl) Bis(phosphorothioate);
O,O,O′,O′-tetrabutyl S,S′-(octane-1,8-diyl) Bis(phosphorothioate);
O,O,O′,O′-tetrabutyl S,S′-(nonane-1,9-diyl) Bis(phosphorothioate);
O,O,O′,O′-tetrabutyl S,S′-(decane-1,10-diyl) Bis(phosphorothioate);
S′,S′-(hexane-1,6-diylbis(azenediyl)) Bis(2-oxoethane-2,1-diyl) O,O,O′,O′-Tetraethyl Diphosphorodithioate;
S′,S′-(butane-1,4-diylbis(azenediyl)) Bis(2-oxoethane-2,1-diyl) O,O,O′,O′-Tetrabutyl Diphosphorodithioate;
S′,S′-(pentane-1,5-diylbis(azenediyl)) Bis(2-oxoethane-2,1-diyl) O,O,O′,O′-Tetrabutyl Diphosphorodithioate;
S′,S′-(hexane-1,6-diylbis(azenediyl)) Bis(2-oxoethane-2,1-diyl) O,O,O′,O′-Tetrabutyl Diphosphorodithioate;
S′,S′-(heptane-1,7-diylbis(azenediyl)) bis(2-oxoethane-2,1-diyl) O,O,O′,O′-Tetrabutyl Diphosphorodithioate;
S′,S′-(octane-1,8-diylbis(azenediyl)) Bis(2-oxoethane-2,1-diyl) O,O,O′,O′-Tetrabutyl Diphosphorodithioate;
S′,S′-(nonane-1,9-diylbis(azenediyl)) Bis(2-oxoethane-2,1-diyl) O,O,O′,O′-Tetrabutyl Diphosphorodithioate;
S′,S′-(decane-1,10-diylbis(azenediyl)) Bis(2-oxoethane-2,1-diyl) O,O,O′,O′-Tetrabutyl Diphosphorodithioate;
Di-n-butyl 4-methylbenzylphosphonate;
Di-n-butyl 4-fluorobenzylphosphonate;
Di-n-butyl 4-methoxybenzylphosphonate;
Di-n-butyl 4-chlorobenzylphosphonate;
Di-n-butyl 1-naphthylmethylphosphonate;
Di-n-butyl benzylphosphonate;
Dimethyl phenyl phosphate;
Diethyl phenyl phosphate;
Di-n-propyl phenyl phosphate;
Di-n-butyl phenyl phosphate;
Di-n-pentyl phenyl phosphate;
Di-cyclohexyl phenyl phosphate; or
(±)-n-Butyl ethyl phenyl phosphate;
   or a pharmaceutically acceptable salt thereof.

In some embodiments, provided herein are compounds described herein for use as described herein.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 depicts inhibition of aggregation of amyloid peptides by tetraethyl hexane-1,6-diyl bis(phosphate) (also referred to as tetraethyl hexyl biphosphonate).

FIG. 2 depicts inhibition of aggregation of amyloid peptides by tetrabutyl hexane-1,6-diyl bis(phosphate) (also referred to as tetrabutyl hexyl biphosphonate).

FIG. 3 depicts inhibition of aggregation of amyloid peptides by tetraphenyl hexane-1,6-diyl bis(phosphate) (also referred to as tetraphenyl hexyl biphosphonate).

FIG. 4 depicts inhibition of aggregation of amyloid peptides by di-n-butyl (2-chlorophenyl) phosphate.

FIG. 5 shows in vivo inhibition of serum butyrylcholinesterase activity by DB2CIPP in transgenic Alzheimer's mice using 10 mg and 20 mg doses of Di-n-butyl 2-chlorophenyl phosphate (DB2CIPP) over 11 months and 13 months.

FIG. 6 shows in vivo inhibition of β-amyloid plaque formation by DB2CIPP in transgenic Alzheimer's mice.

FIG. 7 shows in vivo inhibition of hippocampal butyrylcholinesterase activity by DB2CIPP in transgenic Alzheimer's mice using 10 mg and 20 mg doses of Di-n-butyl 2-chlorophenyl phosphate (DB2CIPP) over 11 months.

FIG. 8 shows serum acetylcholinesterase activity is substantially not altered by DB2CIPP in transgenic Alzheimer's mice using 10 mg and 20 mg doses of Di-n-butyl 2-chlorophenyl phosphate (DB2CIPP) over 11 months and 13 months.

FIG. 9 shows DB2CIPP reduced beta amyloid plaques immunopositive staining in the hippocampus compared to untreated transgenic Alzheimer's mice using 10 mg and 20 mg doses of Di-n-butyl 2-chlorophenyl phosphate (DB2CIPP) over 13 months.

### DETAILED DESCRIPTION

Described herein are organophosphorous compounds that affect the function of esterases and other proteins, which are useful in treating diseases. It has been discovered that the compounds provided herein, acting as selective inhibitors of BCHE over other esterases, including ACHE, find use in treating a variety of diseases including inflammatory diseases, diseases associated with cholinergic dysfunction, autoimmune diseases, and long COVID, among others. For example, while other non-phosphorous compounds may be known to have some selectivity toward BCHE over ACHE, e.g., rivastigmine has approximately 12-fold selectivity (BCHE IC₅₀ of 260 nM vs. ACHE IC₅₀ of 3,030 nM), certain of the compounds provided herein have significantly higher selectivity, e.g., DB2CIPP has approximately 68,000-fold selectivity (BCHE IC₅₀ of 0.69 nM vs. ACHE IC₅₀ of 47,000 nM).

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The term “alkyl” or “alkylene” refers to branched, cyclic, or straight chain, or a combination thereof, saturated hydrocarbon.

The term “alkenyl” refers to an unsaturated hydrocarbon that includes at least one carbon-carbon double bond.

The term “alkynyl” refers to an unsaturated hydrocarbon that includes at least one carbon-carbon triple bond.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The term “aryl” refers to a carbocyclic aromatic system comprising one, two, three, or more rings.

The term “Cₙ₋ₘ” refers to a moiety comprising n to m carbon atoms, wherein n and m are integers.

The terms “composition” and “pharmaceutical composition” refer to a mixture of at least one compound described herein with a pharmaceutically acceptable carrier. The pharmaceutical composition facilitates administration of the compound to a patient or subject. Multiple techniques of administering a compound exist including, but not limited to, intravenous, oral, aerosol, parenteral, ophthalmic, nasal, pulmonary, and topical administration.

The terms “effective amount” and “therapeutically effective amount” refer to an amount of therapeutic compound, such as a compound described herein, administered to a subject, either as a single dose or as part of a series of doses, which is effective to produce a desired therapeutic effect. In general, the therapeutically effective amount can be estimated initially either in cell culture assays or in mammalian animal models, for example, in non-human primates, mice, rabbits, dogs, or pigs. The animal model may also be used to determine the appropriate concentration range and route of administration. Such information can then be used to determine useful doses and routes for administration in non-human subjects and human subjects.

The term “halo” or “halogen” refers to one or more atoms independently selected from F, Br, Cl, or I.

The term “heteroaryl” refers to an aryl moiety comprising at least one ring heteroatom selected from O, S, or N, wherein each ring may comprise, independently, one, two, three, or four ring heteroatoms independently selected from O, S, or N.

The term “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions described herein are known in the art and described, for example, in “Remington's Pharmaceutical Sciences” (Genaro (Ed.), Mack Publishing Co., 1985), the entire content of which is incorporated herein by reference.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

The term “refractory disease” refers to a disease that continues to progress during treatment with a pharmaceutical ingredient other than the compounds provided herein, partially responds to the other treatment, or transiently responds to the other treatment. The term may be applied to each of the diseases referred to herein.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability. When any such inclusion or exclusion occurs, the specification is deemed to contain the group as modified thus fulfilling the written description of all Markush groups used in the appended claims.

References have been made to patents and printed publications throughout this specification, each of which are individually incorporated herein by reference in their entirety.

It is to be understood that the embodiments of this disclosure are illustrative. Accordingly, the present disclosure is not limited to that precisely as shown and described.

### Compounds

It has been found that the compounds described herein are useful as selective inhibitors of esterases, including cholinesterases, e.g., butyrylcholinesterase (BCHE) selectivity over acetylcholinesterase (ACHE).

In some embodiments, the compounds described herein are selected from a compound of Formula 1:

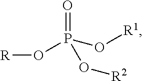

or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl.

In some embodiments, the compounds described herein are selected from a compound of Formula 2:

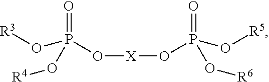

or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl.

In some embodiments, the compounds described herein are selected from a compound of Formula 1A, Formula 1B, Formula 1C, Formula 1D, Formula 2A, Formula 2B, Formula 2C, or Formula 2D:

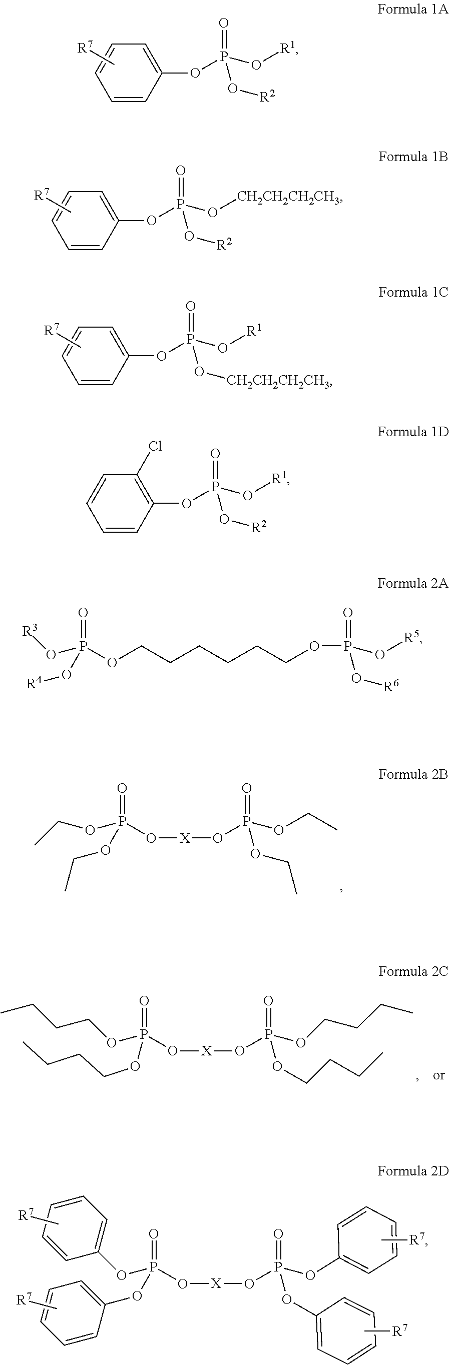

or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷.

In some embodiments, the compounds described herein are selected from a compound of Formula 3, Formula 4, Formula 5, Formula 6, Formula 7, Formula 8, or Formula 9:

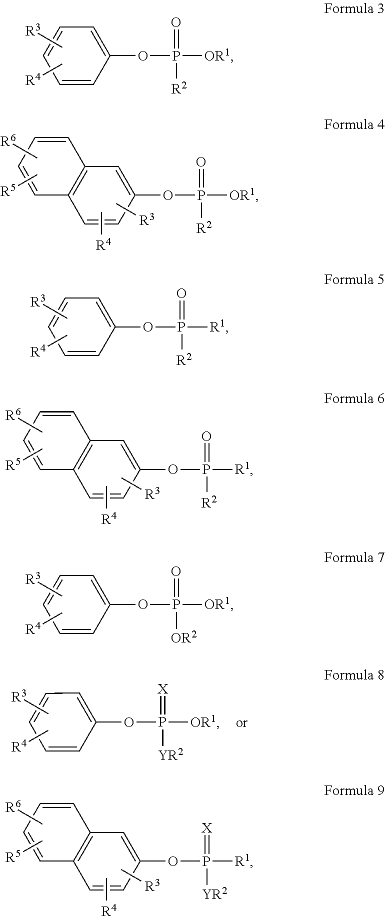

or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂).

In some embodiments of the formulae provided herein, R is optionally substituted C₅₋₁₀ aryl or C₅₋₆ heteroaryl. In some embodiment, R is C₅₋₁₀ aryl. In some embodiments, R is 5- or 6-membered heteroaryl. In some embodiments, R is 2-chlorophenyl, phenyl, or pyridine. In some embodiments, R is 2-chlorophenyl. In some embodiments R is phenyl.

In some embodiments of the formulae provided herein, R¹ is optionally substituted C₁₋₁₂ alkyl. In some embodiments, R¹ is C₁₋₆ alkyl, such as ethyl, propyl, or butyl, etc. In some embodiments, R¹ is n-butyl.

In some embodiments of the formulae provided herein, R² is optionally substituted C₁₋₁₂ alkyl. In some embodiments, R² is C₁₋₆ alkyl, such as ethyl, propyl, or butyl, etc. In some embodiments, R² is n-butyl.

In some embodiments of the formulae provided herein, R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}. In some embodiments, R⁷ is halogen, such as F, Cl, CH₃, OCH₃, CN, OH, —C(O)OH, —C(O)NH₂, —S(O)₂NH₂, —S(O)₂CH₃, NHS(O)₂CH₃, or NH₂, etc. In some embodiments, R⁷ is Cl. In some embodiment, Cl as R⁷ is located at the ortho-position of the phosphate group of the aryl or heteroaryl ring.

In some embodiments of the formulae provided herein, R^{a} and R^{b} are independently H or C₁₋₆ alkyl. In some embodiments, R^{a} and R^{b} are independently C₁₋₆ alkyl, such as CH₃, C₂H₅, C₃H₇, C₄H₉, C₅H₁₁, or C₆H₁₃. In some embodiments, R^{a} and R^{b} are independently H.

In some embodiments of the formulae provided herein, R³, R⁴, R⁵ and R⁶ are independently C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷. In some embodiment, R³, R⁴, R⁵ and R⁶ are independently C₅₋₁₀ aryl. In some embodiments, R³, R⁴, R⁵ and R⁶ are independently C₅₋₆ heteroaryl. In some embodiment, R³, R⁴, R⁵ and R⁶ are independently C₁₋₁₂ alkyl. In some embodiments, R³, R⁴, R⁵ and R⁶ are independently C₁₋₆ alkyl, such as methyl, ethyl, propyl, butyl, pentyl or hexyl. In some embodiments, R³, R⁴, R⁵ and R⁶ are independently ethyl. In some embodiments, R³, R⁴, R⁵ and R⁶ are independently n-butyl. In some embodiments, R³, R⁴, R⁵ and R⁶ are independently phenyl.

In some embodiments of the formulae provided herein, X is C₁₋₁₂ alkyl which can be optionally substituted with R⁷. In some embodiments, X is optionally substituted C₁₋₁₂ alkyl. In some embodiments, X is ethyl, propyl, butyl, pentyl, or hexyl. In some embodiments, X is n-butyl.

Unless otherwise indicated, when a compound or chemical structural feature, such as alkyl, alkenyl, alkynyl, aryl, heteroaryl, etc., is referred to as being “optionally substituted,” it includes a feature that has no substituents (i.e. unsubstituted), or a feature that is “substituted,” meaning that the feature has one or more substituents. Optional substituents include, without limitation, C₁₋₆ alkyl, halo, or haloalkyl.

The compounds referred to herein include those of Table 1, which, in some instances, may be provided as a pharmaceutically acceptable salt thereof.

TABLE 1

| Com- | |
|---|---|
| pound | Name |
| 1 | 2-Bromophenyl di-n-butyl phosphate |
| 2 | 3-Bromophenyl n-dibutyl phosphate |
| 3 | Dibutyl 3-chlorophenyl phosphate |
| 4 | 4-Bromophenyl di-n-butyl phosphate |
| 5 | 2-Chlorophenyl dimethyl phosphate |
| 6 | 2-Chlorophenyl diethyl phosphate |
| 7 | 2-Chlorophenyl di-n-propyl phosphate |
| 8 | 2-Chlorophenyl di-i-propyl phosphate |
| 9 | Di-n-butyl 2-chlorophenyl phosphate |
| 10 | Di-i-butyl 2-Chlorophenyl phosphate |
| 11 | 2-Chlorophenyl di-n-pentyl phosphate |
| 12 | 2-Chlorophenyl dicyclohexyl phosphate |
| 13 | Di-n-butyl 4-chlorophenyl phosphate |
| 14 | Di-n-butyl 2,4-dichlorophenyl phosphate |
| 15 | Di-n-butyl 2-fluorophenyl phosphate |
| 16 | Di-n-butyl 3-fluorophenyl phosphate |
| 17 | Dibutyl 2-chlorophenyl thiophosphate |
| 18 | n-Butyl bis(2,4-dichlorophenyl) phosphate |
| 19 | Butyl bis(2-chlorophenyl) phosphate |
| 20 | 2-Chlorophenyl diphenyl phosphate |
| 21 | Di-n-butyl 4-nitrophenyl phosphate |
| 22 | Di-n-butyl 3-nitrophenyl phosphate |
| 23 | Di-n-butyl 2-methylphenyl phosphate |
| 24 | Di-n-butyl 3-methylphenyl phosphate |
| 25 | Di-n-butyl 4-methylphenyl phosphate |
| 26 | Di-n-butyl 3,4-dimethylphenyl phosphate |
| 27 | Di-n-butyl 3,5-dimethylphenyl phosphate |
| 28 | Diethyl 3,5-dimethylphenyl phosphate |
| 29 | Di-n-butyl 3,4,5-trimethylphenyl phosphate |
| 30 | Benzyl di-n-butyl phosphate |
| 31 | Di-n-butyl 2-methylbenzyl phosphate |
| 32 | Di-n-butyl 3-methylbenzyl phosphate |
| 33 | Di-n-butyl 4-methylbenzyl phosphate |
| 34 | Di-n-butyl 1-napthyl phosphate |
| 35 | Di-n-butyl 2-napthyl phosphate |
| 36 | Diethyl 2-napthyl phosphate |
| 37 | Di-n-butyl 2-phenylethyl phosphate |
| 38 | Butyl 2-chlorophenyl hexylphosphonate |
| 39 | Bis(2-chlorophenyl) ethylphosphonate |
| 40 | Butyl 2-chlorophenyl ethylphosphonate |
| 41 | Bis(3-nitrophenyl) ethylphosphonate |
| 42 | Butyl p-nitrophenyl ethylphosphonate |
| 43 | Bis (p-nitrophenyl) ethylphosphonate |
| 44 | Bis(2-chlorophenyl) hexylphosphonate |
| 45 | Bis(2-chlorophenyl) n-butylphosphonate |
| 46 | Bis(2-chlorophenyl) ethylphosphonate |
| 47 | 2-Chlorophenyl di-n-pentylphosphinate |
| 48 | 2-Chlorophenyl di-n-butylphosphinate |
| 49 | Phenyl di-n-pentylphosphinate |
| 50 | 4-nitrophenyl dipentylphosphinate |
| 51 | O-n-butyl-O-2-chlorophenyl-N-hexyl phosphoramidate |
| 52 | Bis(2-chlorophenyl) N-hexyl phosphoramidate |
| 53 | Tetraethyl propane-1,3-diyl bisphosphate |
| 54 | Tetraethyl butane-1,4-diyl bisphosphate |
| 55 | Tetraethyl pentane-1,5-diyl bisphosphate |
| 56 | Tetraethyl hexane-1,6-diyl bisphosphate |
| 57 | Tetraethyl heptane-1,7-diyl bisphosphate |
| 58 | Tetraethyl 3-oxapentane-1,5-diyl bisphosphate |
| 59 | Tetrabutyl propane-1,3-diyl bisphosphate |
| 60 | Tetrabutyl butane-1,4-diyl bisphosphate |
| 61 | Tetrabutyl pentane-1,5-diyl bisphosphate |
| 62 | Tetrabutyl hexane-1,6-diyl bisphosphate |
| 63 | Tetrabutyl heptane-1,7-diyl bisphosphate |
| 64 | Tetrabutyl octane-1,8-diyl bisphosphate |
| 65 | Tetrabutyl 3-oxapentane-1,5-diyl bisphosphate |
| 66 | Tetrabutyl 3-hexene-1,6-diyl bisphosphate |
| 67 | Tetraphenyl hexane-1,6-diyl bisphosphate |
| 68 | O,O,O′,O′-tetrabutyl S,S′-(ethane-1,2-diyl) Bis(phosphorothioate) |
| 69 | O,O,O′,O′-tetrabutyl S,S′-(propane-1,3-diyl) |
| | Bis(phosphorothioate) |
| 70 | O,O,O′,O′-tetrabutyl S,S′-(butane-1,4-diyl) |
| | Bis(phosphorothioate) |
| 71 | O,O,O′,O′-tetrabutyl S,S′-(pentane-1,5-diyl) |
| | Bis(phosphorothioate) |
| 72 | O,O,O′,O′-tetrabutyl S,S′-(hexane-1,6-diyl) |
| | Bis(phosphorothioate) |
| 73 | O,O,O′,O′-tetrabutyl S,S′-(heptane-1,7-diyl) |
| | Bis(phosphorothioate) |
| 74 | O,O,O′,O′-tetrabutyl S,S′-(octane-1,8-diyl) Bis(phosphorothioate) |
| 75 | O,O,O′,O′-tetrabutyl S,S′-(nonane-1,9-diyl) |
| | Bis(phosphorothioate) |
| 76 | O,O,O′,O′-tetrabutyl S,S′-(decane-1,10-diyl) |
| | Bis(phosphorothioate) |
| 77 | S′,S′-(hexane-1,6-diylbis(azenediyl)) Bis(2-oxoethane-2,1-diyl) |
| | O,O,O′,O′-Tetraethyl Diphosphorodithioate |
| 78 | S′,S′-(butane-1,4-diylbis(azenediyl)) Bis(2-oxoethane-2,1-diyl) |
| | O,O,O′,O′-Tetrabutyl Diphosphorodithioate |
| 79 | S′,S′-(pentane-1,5-diylbis(azenediyl)) Bis(2-oxoethane-2,1-diyl) |
| | O,O,O′,O′-Tetrabutyl Diphosphorodithioate |
| 80 | S′,S′-(hexane-1,6-diylbis(azenediyl)) Bis(2-oxoethane-2,1-diyl) |
| | O,O,O′,O′-Tetrabutyl Diphosphorodithioate |
| 81 | S′,S′-(heptane-1,7-diylbis(azenediyl)) bis(2-oxoethane-2,1-diyl) |
| | O,O,O′,O′-Tetrabutyl Diphosphorodithioate |
| 82 | S′,S′-(octane-1,8-diylbis(azenediyl)) Bis(2-oxoethane-2,1-diyl) |
| | O,O,O′,O′-Tetrabutyl Diphosphorodithioate |
| 83 | S′,S′-(nonane-1,9-diylbis(azenediyl)) Bis(2-oxoethane-2,1-diyl) |
| | O,O,O′,O′-Tetrabutyl Diphosphorodithioate |
| 84 | S′,S′-(decane-1,10-diylbis(azenediyl)) Bis(2-oxoethane-2,1-diyl) |
| | O,O,O′,O′-Tetrabutyl Diphosphorodithioate |
| 85 | Di-n-butyl 4-methylbenzylphosphonate |
| 86 | Di-n-butyl 4-fluorobenzylphosphonate |
| 87 | Di-n-butyl 4-methoxybenzylphosphonate |
| 88 | Di-n-butyl 4-chlorobenzylphosphonate |
| 89 | Di-n-butyl 1-naphthylmethylphosphonate |
| 90 | Di-n-butyl 2-naphthylmethylphosphonate |
| 91 | Di-n-butyl benzylphosphonate |
| 92 | Di-n-butyl 3,5-dimethylbenzylphosphonate |
| 93 | Dimethyl phenyl phosphate |
| 94 | Diethyl phenyl phosphate |
| 95 | Di-n-propyl phenyl phosphate |
| 96 | Di-n-butyl phenyl phosphate |
| 97 | Di-n-pentyl phenyl phosphate |
| 98 | Di-cyclohexyl phenyl phosphate |
| 99 | (±)-n-Butyl ethyl phenyl phosphate |

Compounds described herein also include isotopically-labeled compounds wherein one or more atoms is replaced by an atom having the same atomic number, but an atomic mass or mass number different from the atomic mass or mass number predominantly found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In some embodiments, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In some embodiments, the compounds described herein are labeled by other means, including, but not limited to, the use of chromophores or fluorescent moieties, bioluminescent labels, or chemiluminescent labels.

### Synthesis

Methods of synthesizing the herein described compounds will be evident to those of ordinary skill in the art. Synthetic chemistry transformations and protecting group methodologies (protection and deprotection) useful in synthesizing the compounds are known in the art and include, for example, those such as described in R. LaRock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Accordingly, the compounds may be prepared by a method of synthesis comprising any one or more of Method A, Method B, Method C, Method D, Method E, Method F, Method G, or Method H, described below. Furthermore, the compounds may be prepared analogous to the synthetic sequences described in, for example, U.S. Pat. No. 9,757,399 B2, the entire content of which is incorporated herein by reference.

Representative Procedure for Method A. To a dry 50 mL round bottom flask were added 10 mL of CH₂Cl₂ and 1.3 mL (2.0 g, 8.2 mmol) of 2-chlorophenyl dichlorophosphate. The solution was cooled to 0° C. using an ice water bath. Then 2.5 equivalents of alcohol and 2.5 equivalents of pyridine in 10 mL of CH₂Cl₂ were added to the stirring solution via cannulation. The reaction was left to stir overnight at room temperature. The reaction mixture was diluted with 80 mL of diethyl ether and washed three times with 40 mL of 10% HCl. The aqueous layers were combined and washed with 40 mL of CH₂Cl₂. The combined organic layer was washed once with 40 mL of saturated sodium bicarbonate, dried over magnesium sulfate, filtered, concentrated in vacuo, and purified by evaporative distillation.

Representative Procedure for Method B. A solution of dibutyl phosphite (0.73 g, 3.8 mmol) in 3 mL CCl₄ was added dropwise to a stirring solution containing the substituted phenol (3.0 mmol), 5 mL CCl₄, tetra-n-butylammonium bromide (0.095 g, 0.3 mmol), NaOH (0.18 g, 4.4 mmol), and 5 mL water, and then stirred at room temperature. The reaction mixture was dissolved in 10 mL of CCl₄, extracted with ice-cold distilled water (3×10 mL), and each of the aqueous layer was washed with 10 mL CCl₄. The combined organic layer was dried under MgSO₄, concentrated in vacuo, and purified by evaporative distillation, flash chromatography or gravity chromatography.

Representative Procedure for Method C. General procedure for the preparation of triphosphates: to a dried two or three-necked 150 mL round bottom flask, 1.04 mL (1.14 g, 5.00 mmol) of di-n-butyl chlorophosphate was dissolved in 12 mL CH₂Cl₂ with stirring. The solution was cooled to 0° C. Then 2.0 equivalents of alcohol and 2.0 equivalents of pyridine were added to the solution via syringe. The reaction was stirred overnight at room temperature. The reaction mixture was diluted with 80 mL of diethyl ether and washed three times with 30 mL of 10% HCl. The aqueous layers were combined and washed with 40 mL of CH₂Cl₂. The combined organic layer was washed once with 40 mL of saturated sodium bicarbonate, once with 40 mL of brine, dried over magnesium sulfate, filtered, concentrated in vacuo. The products were purified by flash chromatography.

Representative Procedure for Method D. General procedure for the synthesis of bisphosphates: to a dried two-necked, 50 mL round bottom flask, dialkyl chlorophosphate (12 mmol) was dissolved in 10 mL of CH₂Cl₂ while stirring. The solution was cooled to 0° C. Then pyridine (12 mmol) and diol (4 mmol) were added to the solution via syringe, respectively. The reaction was stirred for 3 days at room temperature. The reaction mixture was diluted with 40 mL of diethyl ether and washed three times with 20 mL of 10% HCl. The aqueous layers were combined and washed with 20 mL of CH₂Cl₂. The combined organic layer was washed once with 20 mL of saturated sodium bicarbonate, once with 20 mL of brine, dried over anhydrous magnesium sulfate, filtered, concentrated in vacuo, and purified by flash chromatography using non-flash silica gel.

Representative Procedure for Method E. Synthesis of O,O,O′,O′-tetrabutyl S,S′-(alkane-α,ω-diyl) Bis(phosphorothioate): dibutyl thiophosphate potassium salt (9 mmol), alkyl dibromide (3 mmol) and 20 mL of dried ethanol were added to a two-neck round-bottom flask equipped with magnetic stirrer, reflux condenser, and rubber septum under a nitrogen atmosphere. The reaction mixture was then heated to reflux and stirred for 1-2 days while monitored periodically through TLC and GC-MS analysis. After cooling to room temperature, the solution was filtered and reduced under pressure. After concentration, the reaction mixture was diluted with 40 mL of diethyl ether and washed three times with 60 mL of water; the aqueous layers were combined and extracted three times with 50 mL of diethyl ether. The combined organic layer was washed with 80 mL of brine and dried over Mg₂SO₄. Evaporation of the solvent afforded a light yellow oil crude material, which was purified by flash column chromatography on flash grade silica gel eluting with varying ratio of hexane and ethyl acetate to afford the final product.

Representative Procedure for Method F. Synthesis of O,O,O′,O′-tetrabutyl S,S′-(alkane-α,ω-diyl) Bis(phosphorothioate): to a flame-dried round-bottom-flask flushed with N₂, NaH (9 mmol) and 1,8 octanedithiol (3 mmol) was stirred and cooled to 0° C. for 20 minutes in 20 mL THF. After 20 minutes the reaction mixture was taken off the ice bath and dibutyl chlorophosphate (9 mmol) was added. The reaction mixture was stirred at room temperature for 1 to 3 days and monitored via TLC and GC-MS. The solution was diluted with 40 mL diethyl ether and washed three times with 60 mL of water. The combined aqueous layer was extracted three times with 50 mL of diethyl ether. The combined organic layer was washed with 80 mL of brine. The collected organic layer was dried with magnesium sulfate, filtered and concentrated under vacuum. The final compound was purified through flash column chromatography using flash grade silica gel.

Representative Procedure for Method G. Synthesis of O,O,O′,O′-tetrabutyl S,S′-(alkane-α,ω-diyl) Bis(phosphorothioate): to a 100-mL round-bottom-flask, flushed with N₂, NaH (9 mmol) and dithiol (4 mmol) were mixed with 20 mL of DMF and cooled to 0° C. for 20 minutes. After 20 minutes the reaction mixture was taken off the ice bath and dibutyl chlorophosphate (12 mmol) was added. The reaction mixture was stirred at room temperature for 1 to 3 days and monitored via TLC and GC-MS. The resulting solution was diluted with 40 mL diethyl ether and washed three times with 60 mL of water. The combined aqueous layer was extracted three times with 50 mL of diethyl ether. The combined organic layer was washed with 80 mL of brine. The collected organic layer was dried over Mg₂SO₄, filtered and concentrated under vacuum. The final compound was purified through flash column chromatography using flash grade silica gel.

Representative Procedure for Method H. Synthesis of S′,S′-(alkane-α,ω-diylbis(azenediyl)) Bis(2-oxoethane-2,1-diyl) O,O,O′,O′-Tetrabutyl Diphosphorodithioate: Dibutyl thiophosphate potassium salt (9 mmol), the corresponding N′,N′-(alkane-α,ω-diyl) bis(2-chloroacetamide) (3 mmol), 5% KI and 20 mL of dry methanol were added to a twoneck round-bottom flask equipped with magnetic stirrer, reflux condenser, and rubber septum under a nitrogen atmosphere. The reaction mixture was then heated to reflux and stirred for 1-2 days while monitored periodically through TLC and visualized in Iodine vapor. After cooling to room temperature, the solution was filtered and concentrated under reduced pressure. After concentration under vacuum, the reaction mixture was diluted with 40 mL of ethyl acetate and washed two times with 40 mL of water. The combined aqueous layer was extracted three times with 50 mL of ethyl acetate. The combined organic layer was washed with 80 mL of brine and dried over Mg₂SO₄. Evaporation of the solvent afforded the crude product as a viscous clear oil, which was purified by flash column chromatography on silica gel of particle size (40-63) μm and varying ratios of ethanol:ethyl acetate or hexane:ethyl acetate as eluents.

Exemplary synthetic methods are described in Table 2.

TABLE 2
Compound 1.

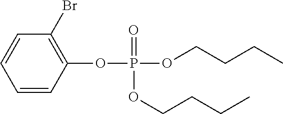 [SMILES: CCCCOP(=O)(OCCCC)Oc1ccccc1Br]

Synthesis of 2-Bromophenyl di-n-butyl phosphate: Following the representative procedure
described in Method B using 2-bromophenol (2 hours reaction time), the crude product was
purified by evaporative distillation (b.p. 175° C./0.45 mm Hg) followed by flash
chromatography (silica gel, hexane:EtOAc, 3:2) to give 2-bromophenyl di-n-butyl
phosphate as a pale yellow oil: 56% yield; R_{f} = 0.49 (hexane:EtOAc, 1:1); ¹H NMR (400
MHz, CDCl₃) δ 0.92 (t, J = 7.4 Hz, 6H, CH₂CH₂CH₂CH₃), 1.36-1.46 (m, 4H, CH₂CH₂CH₂CH₃),
1.65-1.73 (m, 4H, CH₂CH₂CH₂CH₃), 4.17-4.26 (m, 4H, CH₂CH₂CH₂CH₃), 7.02-7.06 (m, 1H,
Ar—H), 7.27-7.31, 1H, Ar—H), 7.47 (dt, J = 8.2 Hz, 1.3 Hz, 1H, Ar—H), 7.58 (dt, J = 8.0 Hz,
1.4 Hz, 1H, Ar—H); ¹³C NMR (100MHz, CDCl₃) δ 13.46, 18.54, 32.07, 32.14, 68.61, 68.68,
114.29, 114.38, 120.99, 121.01, 125.93, 128.55, 128.56, 133.54, 147.87, 147.93.
Compound 2.

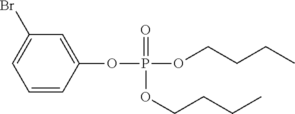 [SMILES: CCCCOP(=O)(OCCCC)Oc1cccc(Br)c1]

Following the representative procedure described in Method B using 3-bromophenol, the
crude product was purified by gravity chromatography (silica gel, hexane:EtOAc, 4:1) to
give 3-bromophenyl di-n-butyl phosphate as a clear oil: 38% yield; R_{f} = 0.5 (hexane:EtOAc,
1:1); ¹H NMR (400MHz, CDCl₃) δ 0.93 (t, J = 7.4 Hz, 6H, OCH₂CH₂CH₂CH₃), 1.36-1.45 (m,
4H, OCH₂CH₂CH₂CH₃), 1.64-1.71 (m, 4H, OCH₂CH₂CH₂CH₃), 4.10-4.20 (m, 4H,
OCH₂CH₂CH₂CH₃), 7.16-7.23 (m, 2H, Ar—H), 7.30-7.33 (m, 1H, Ar—H), 7.39-7.40 (m, 1H,
Ar—H); ¹³C NMR (400 MHz, CDCl₃) δ 13.49, 13.50, 18.58, 32.12, 32.19, 68.45, 68.51,
118.75, 118.80, 122.56, 123.47, 123.52, 128.16, 130.70, 151.21, 151.28.
Compound 3.

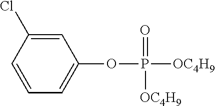

A mixture of tetra-n-butylammonium bromide (1 mmol), aryl alcohol (20 mmol) in CCl₄ and
50% NaOH aqueous solution was stirred at room temperature. To this mixture was added
dropwise a solution of dibutyl phosphite (25 mmol) in CCl₄ in an icewater bath. The reaction
mixture was stirred for 1 hour at room temperature and atmospheric conditions. CCl₄ was
added to quench the reaction. The two layers were extracted with ice-cold water three
times. The collected aqueous layer was washed with CCl₄. The combined organic layer was
dried over anhydrous magnesium sulfate, filtered, and concentrated in vacuo. The products
were purified by flash chromatography on silica gel (ethyl acetate 10%-40%, hexane 90%-
60%) to afford the desired dibutyl aryl phosphate. Dibutyl-3-chlorophenyl phosphate is
obtained as yellow oil: (2.64 g) 41% isolated yield; ¹H NMR (400 MHz, CDCl₃) δ 0.94 (t, J =
7.4 Hz, 6H, (CH₃)₂), 1.36-1.46 (m, 4H, (CH₃CH₂)₂), 1.65-1.72 (m, 4H, (CH₃CH₂CH₂)₂),
4.13-4.19 (m, 4H, (CH₃CH₂CH₂CH₂)₂), 7.13-7.18 (m, 2H, Ph—H), 7.24-7.29 (m, 2H, Ph—H);
); ¹³C NMR (100 MHz, CDCl₃) δ 13.59, 18.69, 32.28, 68.58, 118.44, 120.78, 125.34,
130.48, 135.00, 151.40; HRMS: m/z [MH]+ calcd for C₁₄H₂₃ClO₄P: 321.1017; found
321.1019.
Compound 4.

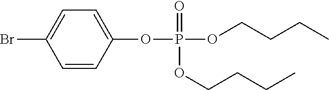 [SMILES: CCCCOP(=O)(OCCCC)Oc1ccc(Br)cc1]

Following the representative procedure described in Method B using 4-bromophenol, the
crude product was purified by gravity chromatography (silica gel, 4:1, hexane:EtOAc) to
give 4-bromophenyl di-n-butyl phosphate as a clear oil: 32% yield; R_{f} = 0.06
(hexane:EtOAc, 4:1); ¹H NMR (400MHz, CDCl₃) δ 0.93 (t, J = 7.4 Hz, 6H, OCH₂CH₂CH₂CH₃),
1.35-1.44 (m, 4H, OCH₂CH₂CH₂CH₃), 1.63-1.71 (m, 4H, OCH₂CH₂CH₂CH₃), 4.08-4.19 (m,
4H, OCH₂CH₂CH₂CH₃), 7.09-7.13 (m, 2H, Ar—H), 7.43-7.47 (m, 2H, Ar—H); ¹³C NMR (400
MHz, CDCl₃) δ 13.49, 13.50, 18.57, 32.13, 32.19, 68.40, 68.46, 117.81, 117.83, 121.74,
121.79, 132.65, 149.86, 149.93.
Compound 5.

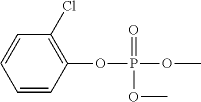

Following the representative procedure described in Method A using methanol, 2-
chlorophenyl dimethyl phosphate was obtained as a clear oil: 80% yield; b.p.183º C./0.2
mm Hg; ¹H NMR (400 MHz, CDCl₃) δ 3.92 (d, 6H, 11.4 Hz, CH₃), 7.11-7.15 (m, 1H, Ar—H),
7.52 (td, 1H, 8.2, 1.7 Hz, Ar—H), 7.41-7.42 (m, 1H, Ar—H), 7.43-7.44 (m, 1H, Ar—H); ¹³C
NMR (100 MHz, CDCl₃) δ 55.22, 55.28, 121.32, 121.34, 125.30, 125.38, 126.03, 128.02,
128.03, 130.65, 146.59, 146.65.
Compound 6.

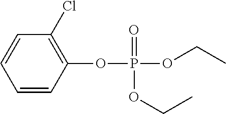 [SMILES: CCOP(=O)(OCC)Oc1ccccc1Cl]

Following the representative procedure described in Method A using ethanol, 2-chlorophenyl
diethyl phosphate was obtained as a clear oil: 100% yield; b.p. 180º C./0.3 mm Hg; ¹H
NMR (400 MHz, CDCl₃) δ 1.37 (td, 6H, 7.1, 1.1 Hz, CH₃), 4.23-4.32 (m, 4H, CH₂), 7.09-
7.13 (m, 1H, Ar—H), 7.24 (td, 1H, 8.2, 1.7 Hz, Ar—H), 7.41 (dt, 1H, 7.9, 1.4 Hz, Ar—H), 7.45
(dt, 1H, 8.2, 1.3 Hz, Ar—H); ¹³C NMR (100 MHz, CDCl₃) δ 16.00, 16.03, 16.07, 16.10, 64.97,
65.03, 121.32, 121.35, 125.32, 125.39, 125.76, 127.88, 127.89, 130.57, 146.77, 146.83.
Compound 7.

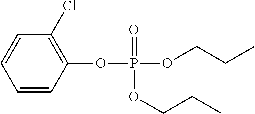 [SMILES: CCCOP(=O)(OCCC)Oc1ccccc1Cl]

Following the representative procedure described in Method A using n-propanol, 2-
chlorophenyl di-n-propyl phosphate was obtained as a clear oil: 82% yield; b.p. 245° C./0.3
mm Hg; ¹H NMR (400 MHz, CDCl₃) δ 0.96 (t, 6H, 7.4 Hz, CH₃), 1.74 (sextet, 4H, 7.3 Hz,
CH₂CH₂ CH₃), 4.11-4.21 (m, 4H, CH₂CH₂ CH₃), 7.08-7,13 (m, 1H, Ar—H), 7.24 (td, 1H, 8.2,
1.7 Hz, Ar—H), 7.41 (dt, 1H, 7.9, 1.3 Hz, Ar—H), 7.46 (dt, 1H, 8.2, 1.3 Hz, Ar—H); ¹³C NMR
(100 MHz, CDCl₃) δ 9.94, 23.56, 23.62, 70.37, 70.44, 121.33, 121.35, 125.34, 125.41,
125.71, 125.73, 127.87, 127.88, 130.55, 146.82, 146.89.
Compound 8.

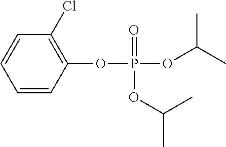 [SMILES: CC(C)OP(=O)(Oc1ccccc1Cl)OC(C)C]

Following the general procedure described in Method A using i-propanol, 2-chlorophenyl di-
i-propyl phosphate was obtained as a clear oil: 70% yield; b.p. 205° C./0.7 mm Hg; ¹H NMR
(400 MHz, CDCl₃) δ 1.33 (d, 6H, 6.2 Hz, CH₃), 1.38 (d, 6H, 6.2 Hz, CH₃), 4.81 (sept of
doublets, 2H, 6.3, 0.8 Hz, CH(CH₃)₂), 7.09 (tm, 1H, 8.0 Hz, Ar—H), 7.23 (td, 1H, 8.2, 1.6
Hz, Ar—H), 7.40 (dt, 1H, 7.9, 1.5 Hz, Ar—H), 7.49 (dt, 1H, 8.2, 1.4 Hz, Ar—H); ¹³C NMR (100
MHz, CDCl₃) δ 23.45, 23.47, 23, 50, 23.52, 23.62, 23.64, 23.67, 23.69, 73.94, 74.00,
121.19, 121.21, 125.27, 125.35, 125.43, 127.75, 127.76, 130.48, 147.01, 147.07.
Compound 9.

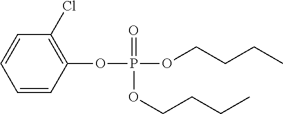 [SMILES: CCCCOP(=O)(OCCCC)Oc1ccccc1Cl]

Following the general procedure described in Method A using n-butanol, di-n-butyl 2-
chlorophenyl phosphate was obtained as a clear oil: 52% yield; b.p. 205° C./0.6 mm Hg;
¹H NMR (400 MHz, CDCl₃) δ 0.92 (t, 6H, 7.4 Hz, CH₃), δ 1.36-1.45 (m, 4H, 6.2, CH₂CH₂CH₂
CH₃), 1,65-1.72 (m, 4H, 6.3, CH₂CH₂CH₂ CH₃), 4.15-4.25 (m, 4H, CH₂CH₂CH₂ CH₃), 7.09-
7.13 (m, 1H, Ar—H), 7.24 (td, 1H, 8.2, 1.6 Hz, Ar—H), 7.41 (dt, 1H, 8.0, 1.4 Hz, Ar—H), 7.45
(dt, 1H, 8.2, 1.3 Hz, Ar-H); ¹³C NMR (100 MHz, CDCl₃) δ 13.53, 18.59, 32.13, 32.20, 68.62,
68.69, 121.32, 121.34, 125.33, 125.41, 125.68, 127.83, 127.84, 130.54, 146.82, 146.88.
Compound 10.

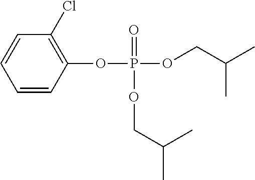 [SMILES: CC(C)COP(=O)(OCC(C)C)Oc1ccccc1Cl]

Following the general procedure described in Method A using i-butanol, di-i-butyl 2-
chlorophenyl phosphate was obtained as clear oil: 40% yield; b.p. 179º C./0.7 mm Hg; ¹H
NMR (400 MHz, CDCl₃) δ 0.95 (appt dd, 12H, 6.7, 1.1 Hz, CH₃), 1.99 (septet, 2H, 6.6 Hz,
CH), 3.92-4.01 (m, 4H, CH₂), 7.08-7.13 (m, 1H, Ar—H), 7.24 (td, 1H, 8.1, 1.7 Hz, Ar—H),
7.41 (dt, 1H, 8.0, 1.4 Hz, Ar—H), 7.46 (dt, 1H, 8.2, 1.2 Hz, Ar—H); ¹³C NMR (100 MHz, CDCl₃)
δ 18.57, 18.59, 18.60, 29.02, 29.09, 74.67, 74.73, 121.37, 121.40, 125.37, 125.44,
125.71, 127.87, 127.88, 130.56, 146.86, 146.91.
Compound 11.

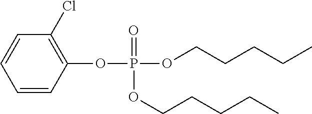 [SMILES: CCCCCOP(=O)(OCCCCC)Oc1ccccc1Cl]

Following the general procedure described in Method A using n-pentanol, 2-chlorophenyl
di-n-pentyl phosphate was obtained as a clear oil: 88%; b.p. 185° C./0.3 mm Hg; ¹H NMR
(400 MHz, CDCl₃) δ 0.89 (t, 6H, 7.2 Hz, CH₃), 1.27-1.39 (m, 8H, CH₂CH₂ CH₃), 1.67-1.74
(m, 4H, CH₂CH₂CH₂CH₃), 4.14-4.24 (m, 4H, CH₂CH₂CH₂CH₂CH₃), 7.08-7.13 (m, 1H, Ar—H),
7.24 (td, 1H, 8.2, 1.6 Hz, Ar—H), 7.41 (dt, 1H, 7.9, 1.4 Hz, Ar—H), 7.46 (dt, 1H, 8.2, 1.3 Hz,
Ar—H); ¹³C NMR (100 MHz, CDCl₃) δ 13.93, 22.17, 27.48, 29.84, 29.90, 68.93, 69.00,
121.33, 121.35, 125.34, 125.41, 125.68, 125.70, 127.85, 127.87, 130.54, 146.81, 146.87.
Compound 12.

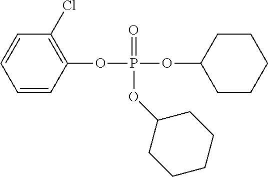 [SMILES: O=P(Oc1ccccc1Cl)(OC1CCCCC1)OC1CCCCC1]

Following the general procedure described in Method A using cyclohexanol, the crude
product was purified by gravity column chromatography (silica gel 1:1, hexane: EtOAc), a
second gravity column chromatography (silica gel 7:3, hexane:EtOAc) and evaporative
distillation (b.p. 260° C./0.10 mm Hg) to afford 2-chlorophenyl dicyclohexyl phosphate as a
colorless oil: 70% yield; ¹H NMR (400 MHz, CDCl₃) δ 1.21-1.98 (m, 20H, (OCHC₅H₁₀)₂),
4.50-4.59 (m, 2H, (OCH)₂), 7.06-7.11 (m, 1H, Ar—H), 7.20-7.25 (m, 1H, Ar—H), 7.38-7.41
(m, 1H, Ar—H), 7.47-7.50 (m, 1H, Ar—H); ¹³C NMR (100 MHz, CDCl₃) δ 23.32, 25.01, 32.97,
33.02, 33.15, 33.19, 78.40, 78.47, 121.16, 121.18, 125.22, 125.31, 127.65, 127.66,
130.37, 147.01, 147.07.
Compound 13.

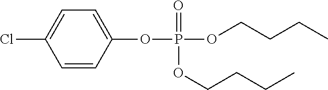 [SMILES: CCCCOP(=O)(OCCCC)Oc1ccc(Cl)cc1]

Following the general procedure described in Method A using dichloro 4-
chlorophenylphosphate and n-butanol, di-n-butyl 4-chlorophenyl phosphate was obtained
as a clear oil: 84%; b.p. 200° C./0.2 mm Hg; ¹H NMR (400 MHz, CDCl₃) δ 0.92 (t, 6H, 7.3
Hz, CH₃), 1.35-1.4 (m, 4H, CH₂CH₂CH₂ CH₃), 1.67 (quintet, 4H, 6.7 Hz, CH₂CH₂CH₂ CH₃),
4.09-4.20 (m, 4H, CH₂CH₂CH₂ CH₃), 7.15-7.18 (m, 2H, Ar—H), 7.28-7.31 (m, 1H, Ar—H);
¹³C NMR (100 MHz, CDCl₃) δ 13.41, 18.51, 32.06, 32.13, 68.31, 68.36, 121.24, 129.57,
130.15, 130.16, 149.23, 149.31.
Compound 14.

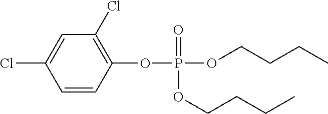 [SMILES: CCCCOP(=O)(OCCCC)Oc1ccc(Cl)cc1Cl]

Following the representative procedure described in Method B, the crude product was
purified by gravity chromatography (silica gel, hexane EtOAc, 9:1) to give di-n-butyl 2,4-
dichlorophenyl phosphate as a light yellow oil: 22.26% yield; R_{f} = 0.12 (silica gel, hexane:
EtOAc, 9:1); ¹H NMR (400MHz, CDCl₃) δ 0.930 (t, J = 7.4 Hz, 6H, OCH₂CH₂H2CH₃)₂), 1.36-
1.46 (m, 4H, (OCH₂CH₂CH₂CH₃)₂), 1.65-1.72 (m, 4H, (OCH₂CH₂CH₂CH₃)₂), 4.14-4.24 (m,
4H, (OCH₂CH₂CH₂CH₃)₂), 7.26 (dd, 1H, J = 8.9, 2.5 Hz, Ph—H), 7.40 (dd, 1H, J = 8.8, 1.1 Hz,
Ph—H), 7.42 (dd, J = 2.6, 1H, 1.1 Hz, Ph—H); ¹³C NMR (100MHz, CDCl₃) δ 13.47, 18.54,
32.08, 32.14, 68.73, 68.80, 122.08, 122.10, 126.21, 126.29, 127.92, 130.20, 130.40,
130.42, 145.60, 145.66.
Compound 15.

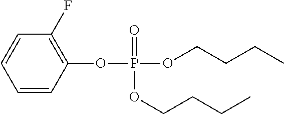 [SMILES: CCCCOP(=O)(OCCCC)Oc1ccccc1F]

Following the representative procedure described in Method B using 2-fluorophenol, the
crude product was purified by gravity chromatography (silica gel, hexane:EtOAc, 4:1) to
give di-n-butyl 2-fluorophenyl phosphate as a clear oil: 48.25% yield; R_{f} = 0.25 (silica gel,
hexane:EtOAc, 4:1); ¹H NMR (400MHz, CDCl₃) δ 0.93 (t, J = 7.4 Hz, 6H, (OCH₂CH₂CH₂CH₃)₂),
1.36-1.46 (m, 4H, (OCH₂CH₂CH₂CH₃)₂), 1.65-1.72 (m, 4H, (OCH₂CH₂CH₂CH₃)₂), 4.13-4.24
(m, 4H, (OCH₂CH₂CH₂CH₃)₂), 7.07-7.17 (m, 3H, Ph—H), 7.35-7.40 (m, 1H, Ph—H); ¹³C NMR
(100MHz, CDCl₃) δ 13.44, 13.48, 18.52, 32.08, 32.15, 68.47, 68.54, 116.72, 116.91,
122.31, 122.34, 124.40, 124.41, 124.44, 124.45, 125.74, 125.75, 125.80, 125.82,
138.36, 138.42,138.48, 138.55, 152.24, 152.30, 154.71, 154.77.
Compound 16.

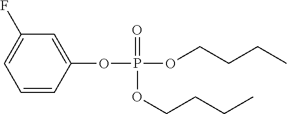 [SMILES: CCCCOP(=O)(OCCCC)Oc1cccc(F)c1]

Following the representative procedure described in Method B using 3-fluorophenol, the
crude product was purified by gravity chromatography (silica gel, hexane:EtOAc, 4:1) to
give di-n-butyl 3-fluorophenyl phosphate as a clear oil: 31% yield; R_{f} = 0.13 (silica gel,
hexane:EtOAc, 4:1); ¹H NMR (400 MHz, CDCl₃) δ 0.93 (t, J = 7.4 Hz, 6H, OCH₂CH₂CH₂CH₃),
1.36-1.45 (m, 4H, OCH₂CH₂CH₂CH₃), 1.64-1.71 (m, 4H, OCH₂CH₂CH₂CH₃), 4.10-4.21 (m,
4H, OCH₂CH₂CH₂CH₃), 6.87-6.92 (m, 1H, Ar—H), 6.97 (dtd, 1H, J = 9.7, 2.3, 0.8 Hz, Ar—H),
7.02-7.04 (m, 1H, Ar—H), 7.29 (td, 1H, 8.2, 6.6 Hz, Ar—H); ¹³C NMR (400 MHz, CDCl₃) δ
13.48, 18.57, 32.12, 32.19, 68.41, 68.48, 107.94, 107.99, 108.19, 108.24, 111.86,
112.07, 115.68, 115.71, 115.73, 115.76, 130.36, 130.44, 151.50, 151.57, 151.67, 161.76,
164.21.
Compound 17.

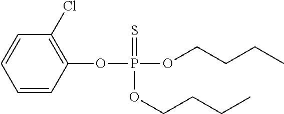 [SMILES: CCCCOP(=S)(OCCCC)Oc1ccccc1Cl]

A mixture containing n-butanol (0.5 mL, 5.4 mmol) and distilled pyridine (0.45 mL, 5.7
mmol) in 5 mL CH₂Cl₂ was added dropwise to 2-chlorophenyl dichlorothiophosphate (0.56
g, 2.1 mmol) dissolved in 15 mL dry CH₂Cl₂. The reaction mixture was allowed to stir for 4
days, followed by dilution with 15 mL CH₂Cl₂, and extraction with 10% HCl (1 x 20 mL) and
saturated NaHCO₃ (3 x 15 mL). The organic layer was dried under MgSO₄, concentrated in
vacuo, and purified by evaporative distillation to yield a pale yellow oil; 73% yield: b.p. 145°
C./0.1 mm Hg; ¹H NMR (400 MHz, CDCl₃) δ 0.94 (t, J = 7.4 Hz, 6H, CH₂CH₂CH₂CH₃), 1.39-
1.48 (m, 4H, OCH₂CH₂CH₂CH₃), 1.68-1.75 (m, 4H, CH₂CH₂CH₂CH₃), 4.22 (dt, J = 8.8 Hz,
6.5 Hz, 4H, CH₂CH₂CH₂CH₃), 7.10-7.14 (m, 1H, Ar—H), 7.22-7.26 (m, 1H, Ar—H), 7.37 (dt,
J = 8.2 Hz, 1.5 Hz, 1H, Ar—H), 7.42 (dt, J = 8.0 Hz, 1.3 Hz, 1H, Ar—H); ¹³C NMR (100MHz,
CDCl₃) δ 13.55, 18.70, 31.95, 32.03, 68.93, 69.00, 122.19, 122.22, 125.82, 125.84,
126.11, 126.18, 127.53, 127.54, 130.51, 146.97, 147.04.
Compound 18.

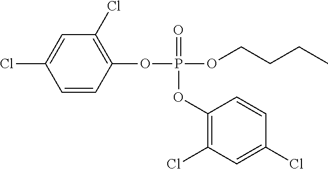 [SMILES: CCCCOP(=O)(Oc1ccc(Cl)cc1Cl)Oc1ccc(Cl)cc1Cl]

To a round bottom flask charged with pyridine (0.06 mL, 0.00073 mol) in 2 mL dry CH₂Cl₂
was added n-butyl alcohol (0.067 mL, 0.00073 mol). This solution was added drop-wise via
cannulation at 0° C. to a solution of bis(2,4dichlorophenyl) phosphorochloridate (0.15 g,
0.00036 mol) in 3 mL dry CH₂Cl₂. The reaction was allowed to react for 24 hrs at room
temperature. The reaction was quenched by extracting with 2 mL 10% HCl, 5 mL saturated
NaHCO₃ and 10 mL ethyl ether. The aqueous phase was extracted 3X5 mL with diethyl
ether. The combined organic layer was dried over MgSO₄, filtered and concentrated in
vacuo. The crude product was purified by gravity column chromatography (silica gel, EtOAc)
to give n-butyl bis(2,4-dichlorophenyl) phosphate as an oil: 10% yield; R_{f} = 0.36 (EtOAc);
¹H NMR (400 MHz, CDCl₃) δ 0.93 (t, 3H, J = 7.6Hz, CH₃), 1.36-1.46 (m, 2H, CH₂CH₂CH₂CH₃),
1.69-1.76 (m, 2H, CH₂CH₂CH₂CH₃), 4.37 (q, 2H, J = 6.8 Hz, CH₂CH₂CH₂CH₃), 7.23 (dd, J =
8.8, 2.4 Hz, 1H, Ar—H), 7.40 (dd, 1H, J = 8.8, 1.2 Hz, Ar—H), 7.44 (dd, 1H, J = 2.8, 1.2 Hz,
Ar—H); ¹³C NMR (100 MHz, CDCl₃) δ 13.44, 18.49, 31.98, 32.05, 70.35, 70.43,
122.23, 122.26, 126.41, 126.48, 128.04, 128.05, 130.42, 131.18, 131.20, 145.14, 145.20.
Compound 19.

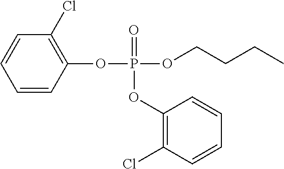 [SMILES: CCCCOP(=O)(Oc1ccccc1Cl)Oc1ccccc1Cl]

A mixture containing n-butanol (0.17 mL, 1.86 mmol), and distilled pyridine (0.15 mL, 1.86
mmol) in 5 mL CH₂Cl₂ was added dropwise to bis(2-chlorophenyl) chlorophosphate (0.63 g,
1.56 mmol) dissolved in 15 mL dry CH₂Cl₂. The reaction mixture was allowed to stir for 24
hours, followed by dilution with 15 mL CH₂Cl₂, extraction with 10% HCl (1 x 20 mL) and
saturated NaHCO₃ (3 x 15 mL), and was dried under MgSO₄. Upon concentration in vacuo,
a pale yellowish oil was obtained in 77% yield: ¹H NMR (400 MHz, CDCl₃) 0 0.92 (t, J = 7.4
Hz, 3H, CH₂CH₂CH₂CH₃), 1.37-1.46 (m, 2H, CH₂CH₂CH₂CH₃), 1.70-1.77 (m, 2H,
CH₂CH₂CH₂CH₃), 4.37-4.42 (m, 2H, CH₂CH₂CH₂CH₃), 7.11-7.16 (m, 2H, Ar—H), 7.23 (dd,
J = 8.2 Hz, 1.7 Hz, 2H, Ar—H), 7.42 (dt, J = 7.9 Hz, 1.4 Hz, 2H, Ar—H), 7.47 (dt, J = 8.2Hz,
1.4 Hz, 2H, Ar—H); ¹³C NMR (100MHz, CDCl₃) δ 13.45, 18.49, 32.01, 32.07, 69.97, 70.05,
121.49, 121.51, 125.47, 125.55, 126.14, 126.16, 127.87, 127.88, 130.63, 146.50, 146.56.
Compound 20.

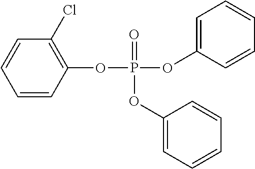 [SMILES: O=P(Oc1ccccc1)(Oc1ccccc1)Oc1ccccc1Cl]

To a round bottom flask charged with NaH (0.1662 g of a 60% dispersion in mineral oil,
approximately 0.42 mmol) in 3 mL dry THF was added phenol (0.383 g, 0.40 mmol) in 2
mL dry THF. Then this solution was added drop wise via cannulation to a solution of 2-
chlorophenyl dichlorophosphate (0.5 g, 0.20 mmol) in 5 mL dry THF at room temperature.
The mixture was allowed to react overnight and quenched by adding 10 mL of saturated
NaHCO₃ and 10 mL of diethyl ether. The aqueous layer was extracted with diethyl ether
(3X 10 mL). The combined organic layer was dried over MgSO₄, filtered and concentrated in
vacuo. The crude product was purified by gravity column chromatography (silica gel,
hexane:EtOAc, 7:3) to give 2-chlorophenyl diphenyl phosphate as an orange oil: 10% yield;
R_{f} = 0.22 (silica, hexane:EtOAc, 7:3); ¹H NMR (400 MHz, CDCl₃) δ 7.14 (t, 1H, J = 8 Hz,
Ar—H), 7.22 (7, 3H, J = 7.2 Hz, Ar—H), 7.28 (d, 4H, J = 8Hz, Ar—H), 7.36 (t, 4H, J = 8 Hz,
Ar—H), 7.41-7.44 (m, 2H, Ar—H); ¹³C NMR (100 MHz, CDCl₃) δ 120.18, 120.23, 121.36, 121.39,
125.50, 125.58, 125.70, 125.71, 126.31, 127.92, 127.93, 129.83, 130.77, 146.53, 146.59,
150.29, 150.37.
Compound 21.

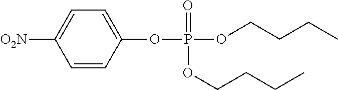 [SMILES: CCCCOP(=O)(OCCCC)Oc1ccc([N+](=O)[O-])cc1]

Following the representative procedure described in Method B using 4-nitrophenol (1 hour
reaction time), the crude product was purified by gravity chromatography (silica gel,
hexane:EtOAc, 3:2) to give di-n-butyl 4-nitrophenyl phosphate as a pale yellow oil: 41%
yield; R_{f} = 0.31 (hexane:EtOAc, 3:2); ¹H NMR (400 MHz, CDCl₃) δ 0.93 (t, J = 7.4 Hz, 6H,
CH₂CH₂CH₂CH₃), 1.36-1.45 (m, 4H, CH₂CH₂CH₂CH₃), 1.66-1.73 (m, 4H, CH₂CH₂CH₂CH₃),
4.13-4.23 (m, 4H, CH₂CH₂CH₂CH₃), 7.36-7.40 (m, 2H, Ar—H), 8.23-8.27 (m, 2H, Ar—H); ¹³C
NMR (100MHz, CDCl₃) δ 13.46, 18.56, 32.09, 32.16, 68.79, 68.85, 120.47, 120.51, 125.65,
144.60, 155.58, 155.65.
Compound 22.

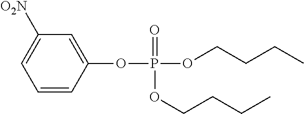 [SMILES: CCCCOP(=O)(OCCCC)Oc1cccc([N+](=O)[O-])c1]

Following the representative procedure described in Method B, the crude product was
purified by gravity chromatography (silica gel, hexane: EtOAc, 4:1) to give di-n-butyl 3-
nitrophenyl phosphate as a light yellow oil: 53% yield; R_{f} = 0.18 (silica gel, hexane:EtOAc,
4:1); ¹H NMR (400MHz, CDCl₃) δ 0.94 (t, J = 7.3 Hz, 6H, (OCH₂CH₂CH₂CH₃)₂), 1.37-1.46 (m,
4H, (OCH₂CH₂CH₂CH₃)₂), 1.67-1.7 (m, 4H, (OCH₂CH₂CH₂CH₃)₂), 4.14-4.24 (m, 4H,
(OCH₂CH₂CH₂CH₃)₂), 7.51-7.56 (m, 1H, Ar—H), 7.61 (ddt, 1H, J = 8.2, 2.2, 1.1 Hz, Ar—H),
8.05-8.08 (m, 2H, Ph—H); ¹³C NMR (100MHz, CDCl₃) δ 13.37, 13.41, 18.49, 32.03, 32.10,
68.67, 68.73, 115.48, 115.53, 119.79, 126.26, 126.31, 130.30, 148.85, 151.10, 151.17.
Compound 23.

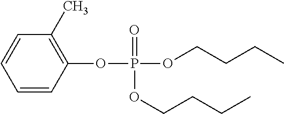 [SMILES: CCCCOP(=O)(OCCCC)Oc1ccccc1C]

Following the general procedure described in Method C and using o-methylphenol, di-n-
butyl 2-methylphenyl phosphate was obtained as a light yellow oil: 37% yield. ¹H NMR (300
MHz, CDCl₃) δ 0.91 (t, J = 7.4 Hz, 6H, (OCH₂CH₂CH₂CH₃)₂), 1.33-1.45 (m, 4H,
(OCH₂CH₂CH₂CH₃)₂), 1.62-1.71 (m, 4H, (OCH₂CH₂CH₂— CH₃)₂), 2.31 (s, 3H, Ph—CH₃), 4.09-
4.19 (m, 4H, (OCH₂CH₂CH₂CH₃)₂), 7.03-7.29 (m, 4H, Ar—H); ¹³C NMR (75 MHz, CDCl₃) δ
15.53, 16.34, 18.62, 32.19, 68.26, 119.70, 124.86, 126.96, 129.25, 131.26,149.22. MS
(gc/ms) m/z (%): 300 (M⁺, 45.9), 188 (100), 107 (100). HRMS: m/z calculated for
C₁₅H₂₆O₄P (MH⁺) 301.1563. Found 301.1521.
Compound 24.

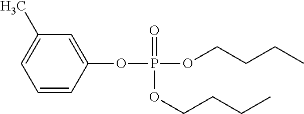 [SMILES: CCCCOP(=O)(OCCCC)Oc1cccc(C)c1]

Following the general procedure described in Method C and using m-methylphenol, di-n-
butyl 3-methylphenyl phosphate was obtained as a light yellow oil: 39% yield. ¹H NMR (300
MHz, CDCl₃) δ 0.84 (t, J = 7.4 Hz, 6H, (OCH₂CH₂CH₂CH₃)₂), 1.26-1.38 (m, 4H,
(OCH₂CH₂CH₂CH₃)₂), 1.55-1.64 (m, 4H, (OCH₂—CH₂CH₂CH₃)₂), 2.26 (s, 3H, Ph—CH₃), 4.03-
4.10 (m, 4H, (OCH₂CH₂—CH₂CH₃)₂), 6.88-7.21 (m, 4H, Ar—H); ¹³C NMR (75 MHz, CDCl₃) δ
13.53, 18.61, 21.30, 32.16, 68.22, 116.84, 120.59, 125.67, 129.31, 139.86,150.67. MS
(gc/ms) m/z (%): 300 (M⁺, 43.7), 188 (100), 108 (49.2). HRMS: m/z calculated for
C₁₅H₂₆O₄P (MH⁺) 301.1563. Found 301.1531.
Compound 25.

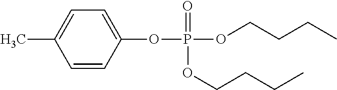 [SMILES: CCCCOP(=O)(OCCCC)Oc1ccc(C)cc1]

Following the general procedure described in Method C and using p-methylphenol, di-n-
butyl 4-methylphenyl phosphate was obtained as a colorless oil: 40% yield. ¹H NMR (300
MHz, CDCl₃) δ 0.92 (t, J = 7.4 Hz, 6H, (OCH₂CH₂CH₂CH₃)₂), 1.33-1.46 (m, 4H,
(OCH₂CH₂CH₂CH₃)₂), 1.62-1.71 (m, 4H, (OCH₂CH₂CH₂—CH₃)₂), 2.31 (s, 3H, Ph—CH₃), 4.09-
4.18 (m, 4H, (OCH₂CH₂CH₂CH₃)₂), 7.07-7.14 (m, 4H, Ar—H); ¹³C NMR (75 MHz, CDCl₃) δ
13.54, 18.62, 20.71, 32.17, 68.14, 119.71, 130.10, 134.48, 148.64. MS (gc/ms) m/z (%):
300 (M⁺, 38.6), 188 (100), 108 (76.8). HRMS: m/z calculated for C₁₅H₂₆O₄P (MH⁺)
301.1563. Found 301.1528.
Compound 26.

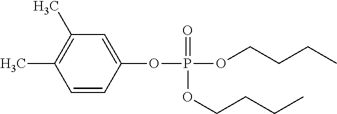 [SMILES: CCCCOP(=O)(OCCCC)Oc1ccc(C)c(C)c1]

Following the general procedure described in Method C and using 3,4-dimethylphenol, di-
n-butyl 3, 4-dimethylphenyl phosphate was obtained as a light yellow oil: 39% yield. ¹H
NMR (300 MHz, CDCl₃) δ 0.92 (t, J = 7.4 Hz, 6H, (OCH₂CH₂CH₂—CH₃)₂), 1.34-1.46 (m, 4H,
(OCH₂CH₂CH₂CH₃)₂), 1.62-1.71 (m, 4H, (OCH₂CH₂CH₂CH₃)₂), 2.21 (s, 3H, Ph—CH₃), 2.23
(s, 3H, Ph—CH₃), 4.09-4.16 (m, 4H, (OCH₂CH₂CH₂CH₃)₂), 6.92-7.07 (m, 3H, Ar—H); ¹³C NMR
(75 MHz, CDCl₃) δ 14.12, 19.18, 20.45, 3.1.51, 68.63, 69.01, 117.49, 121.46, 130.96,
133.68, 138.65, 149.17. MS (GC/MS) m/z (%): 314 (M⁺, 71), 258 (71), 203 (100). HRMS:
m/z calculated for C16H28O4P (MH⁺) 315.1720. Found 315.1663.
Compound 27.

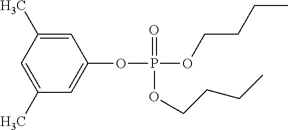 [SMILES: CCCCOP(=O)(OCCCC)Oc1cc(C)cc(C)c1]

Following the general procedure described in Method C and using 3,5-dimethylphenol, di-
n-butyl 3,5-dimethylphenyl phosphate was obtained as a yellow oil: 43% yield. ¹H NMR
(400 MHz, CDCl₃) δ 0.89 (t, J = 7.4 Hz, 6H, (OCH₂CH₂CH₂—CH₃)₂), 1.33-1.42 (m, 4H,
(OCH₂CH₂CH₂CH₃)₂), 1.61-1.68 (m, 4H, (OCH₂CH₂CH₂CH₃)₂), 2.26 (s, 6H, Ph—(CH₃)₂),
4.08-4.13 (m, 4H, (OCH₂CH₂CH₂CH₃)₂), 6.76 (s, 1H, Ar—H), 6.81 (s, 2H, Ar—H); ¹³C NMR
(100 MHz, CDCl₃) δ 11.66, 16.79, 19.35, 30.43, 66.31, 115.73, 124.72, 137.61, 148.88.
MS (gc/ms) m/z (%): 314 (M⁺, 8.4), 202 (100), 122 (41.4). HRMS: m/z calculated for
C₁₆H₂₈O₄P (MH⁺) 315.1720. Found 315.0501.
Compound 28.

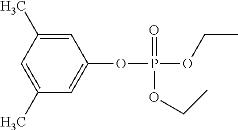 [SMILES: CCOP(=O)(OCC)Oc1cc(C)cc(C)c1]

Following the general procedure described in Method C and using diethyl chlorophosphite
(10) and 3, 5-dimethylphenol, diethyl 3,5-dimethylphenyl phosphate was obtained as a
yellow oil: 39% yield. ¹H NMR (400 MHz, CDCl₃) δ 1.37 (t, J = 7.0 Hz, 6H, (OCH₂CH₃)₂),
2.31 (s, 6H, Ph—(CH₃)₂), 4.18-4.26 (m, 4H, (OCH₂—CH₃)₂), 6.81 (s, 1H, Ar—H), 6.84 (s, 2H,
Ar—H); ¹³C NMR (100 MHz, CDCl₃) δ 16.23, 21.43, 64.60, 117.61, 126.80, 139.67, 150.59.
MS (GC/MS) m/z (%): 258.1 (M⁺, 29.6), 132.1 (100), 122.1 (90.8). HRMS: m/z calculated
for C₁₂H₂₀O₄P (MH+) 259.1094. Found 259.1127.
Compound 29.

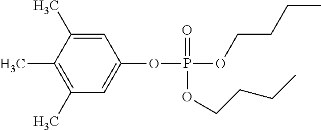 [SMILES: CCCCOP(=O)(OCCCC)Oc1cc(C)c(C)c(C)c1]

Following the general procedure described in Method C and using 3,4,5-trimethylphenol,
di-n-butyl 3,4,5-trimethylphenyl phosphate was obtained as a light yellow oil: 28% yield.
¹H NMR (300 MHz, CDCl₃) δ 0.92 (t, J = 7.4 Hz, 6H, (OCH₂CH₂CH₂—CH₃)₂), 1.34-1.47 (m,
4H, (OCH₂CH₂CH₂CH₃)₂), 1.62-1.72 (m, 4H, (OCH₂CH₂CH₂CH₃)₂), 2.11 (s, 3H Ph—CH₃), 2.25
(s, 6H, Ph—(CH₃)₂), 4.09-4.16 (m, 4H, (OCH₂CH₂CH₂CH₃)₂), 6.86 (s, 2H, Ar—H); ¹³C NMR
(100 MHz, CDCl₃) δ 13.72, 15.00, 18.75, 20.85, 32.34, 68.24, 118.81, 131.83, 137.96,
147.95. MS (GC/MS) m/z (%): 328 (M⁺, 25), 216 (100), 91 (63). HRMS: m/z calculated
for C₁₇H₃₀O₄P (MH⁺) 329.1876. Found 329.1846.
Compound 30.

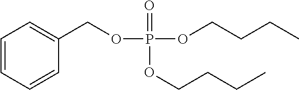 [SMILES: CCCCOP(=O)(OCCCC)OCc1ccccc1]

Following the general procedure described in Method C and using benzyl alcohol, benzyl di-
n-butyl phosphate was obtained as a light yellow oil: 33% yield. ¹H NMR (300 MHz, CDCl₃)
δ 0.91 (t, J = 7.2 Hz, 6H, (OCH₂CH₂CH₂CH₃)₂), 1.32-1.44 (m, 4H, (OCH₂CH₂CH₂CH₃)₂),
1.58-1.67 (m, 4H, (OCH₂CH₂CH₂CH₃)₂), 3.98-4.06 (m, 4H, (OCH₂CH₂CH₂CH₃)₂), 5.07 (d,
J = 8.1 Hz, 2H, (OCH₂—Ph), 7.35-7.42 (m, 5H, Ph—H); ¹³C NMR (75 MHz, CDCl₃) δ 13.57,
18.64, 32.28, 67.58, 69.05, 127.86, 128.55, 136.18, 140.52. MS (gc/ms) m/z (%): 300
(M⁺, 25.4), 188 (100), 91 (77.5). HRMS: m/z calculated for C₁₅H₂₆O₄P (MH⁺) 301.1563.
Found 301.1551.
Compound 31.

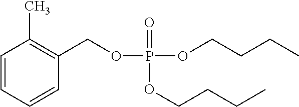 [SMILES: CCCCOP(=O)(OCCCC)OCc1ccccc1C]

Following the general procedure described in Method C and using o-methylbenzyl alcohol,
di-n-butyl 2-methylbenzyl phosphate was obtained as a colorless oil: 42% yield. ¹H NMR
(300 MHz, CDCl₃) δ 0.92 (t, J = 7.4 Hz, 6H, (OCH₂CH₂CH₂CH₃)₂), 1.32-1.44 (m, 4H,
(OCH₂CH₂CH₂CH₃)₂), 1.58-1.68 (m, 4H, (OCH₂— CH₂CH₂CH₃)₂), 2.38 (s, 3H, Ph—CH₃), 3.97-
4.06 (m, 4H, (OCH₂CH₂—CH₂CH₃)₂), 5.09 (d, J = 7.5 Hz, 2H, (OCH₂—Ar), 7.18-7.40 (m, 4H,
Ar—H); ¹³C NMR (75 MHz, CDCl₃) δ 11.57, 18.65, 32.20, 67.46, 126.02, 128.70,
128.86134.08, 136.76. MS (gc/ms) m/z (%): 314 (M⁺, 4.0), 201 (27.2), 105 (100). HRMS:
m/z calculated for C₁₆H₂₈O₄P (MH⁺) 315.1720. Found 315.1724.
Compound 32.

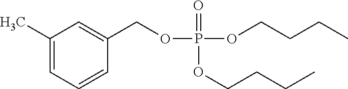 [SMILES: CCCCOP(=O)(OCCCC)OCc1cccc(C)c1]

Following the general procedure described in Method C and using m-methylbenzyl alcohol,
di-n-butyl 3-methylbenzyl phosphate was obtained as a light yellow oil: 40% yield. ¹H NMR
(400 MHz, CDCl₃) δ 0.90 (t, J = 7.4 Hz, 6H, (OCH₂CH₂CH₂CH₃)₂), 1.32-1.42 (m, 4H,
(OCH₂CH₂CH₂CH₃)₂), 1.58-1.65 (m, 4H, (OCH₂—CH₂CH₂CH₃)₂), 2.35 (s, 3H, Ph—CH₃), 3.95-
4.04 (m, 4H, (OCH₂CH₂—CH₂CH₃)₂), 5.02 (d, J = 8.0 Hz, 2H, (OCH₂—Ar), 7.15-7.27 (m, 4H,
Ar—H); ¹³C NMR(100 MHz, CDCl₃) δ 11.72, 16.83, 19.50, 30.47, 65.71, 67.27, 123.09,
126.71, 126.78, 127.32, 134.31, 136.41. MS (gc/ms) m/z (%): 314 (M⁺, 7.9), 202 (60.5),
105 (100). HRMS: m/z calculated for C₁₆H₂₈O₄P (MH⁺) 315.1720. Found 315.1720.
Compound 33.

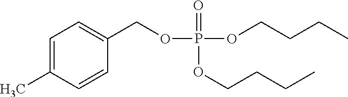 [SMILES: CCCCOP(=O)(OCCCC)OCc1ccc(C)cc1]

Following the general procedure described in Method C and using p-methylbenzyl alcohol,
di-n-butyl 4-methylbenzyl phosphate was obtained as a red-orange oil: 36% yield. ¹H NMR
(300 MHz, CDCl₃) δ 0.92 (t, J = 7.4 Hz, 6H, (OCH₂CH₂CH₂CH₃)₂), 1.32-1.44 (m, 4H,
(OCH₂CH₂CH₂CH₃)₂), 1.58-1.67 (m, 4H, (OCH₂—CH₂CH₂CH₃)₂), 2.36 (s, 3H, Ph—CH₃), 3.97-
4.05 (m, 4H, (OCH₂CH₂—CH₂CH₃)₂), 5.03 (d, J = 8.4 Hz, 2H, (OCH₂—Ar), 7.17-7.30 (m, 4H,
Ar—H); ¹³C NMR(100 MHz, CDCl₃) δ 13.58, 18.64, 21.21, 32.29, 67.44, 69.05, 128.06,
129.21, 133.19, 138.32. MS (gc/ms) m/z (%): 314 (M⁺, 10.4), 202 (100), 121 (79.7).
HRMS: m/z calculated for C₁₆H₂₈O₄P (MH⁺) 315.1720. Found 315.1732.
Compound 34.

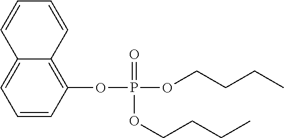 [SMILES: CCCCOP(=O)(OCCCC)Oc1cccc2ccccc12]

Following the general procedure described in Method C and using 1-naphthol, di-n-butyl 1-
naphthyl phosphate was obtained as an orange oil: 48% yield. ¹H NMR (400 MHz, CDCl₃) δ
0.87 (t, J = 7.4 Hz, 6H, (OCH₂CH₂CH₂CH₃)₂), 1.31-1.41 (m, 4H, (OCH₂CH₂—CH₂CH₃)₂),
1.61-1.68 (m, 4H, (OCH₂CH₂CH₂CH₃)₂), 4.13-4.24 (m, 4H, (OCH₂CH₂CH₂CH₃)₂), 7.38-7.19
(m, 7H, Naph—H); ¹³C NMR (100 MHz, CDCl₃) δ 11.65, 16.78, 30.46, 66.62, 113.09,119.83,
122.96, 123.67, 124.60, 124.67, 124.77, 125.92, 132.96, 144.96. MS (gc/ms) m/z (%):
336 (M⁺, 100), 224 (93.6), 144 (48.9). HRMS: m/z calculated for C₁₈H₂₆O₄P (MH⁺)
337.1563. Found 337.1552.
Compound 35.

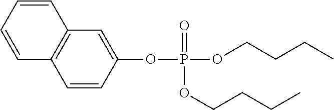 [SMILES: CCCCOP(=O)(OCCCC)Oc1ccc2ccccc2c1]

Following the general procedure described in Method C and using 2-naphthol, di-n-butyl 2-
naphthyl phosphate was obtained as a dark yellow oil: 36.8% yield. ¹H NMR (300 MHz,
CDCl₃) δ 0.93 (t, J = 7.4 Hz, 6H, (OCH₂CH₂CH₂CH₃)₂), 1.36-1.48 (m, 4H, (OCH₂—
CH₂CH₂CH₃)₂), 1.66-1.75 (m, 4H, (OCH₂CH₂CH₂CH₃)₂), 4.16-4.25 (m, 4H,
(OCH₂CH₂CH₂CH₃)₂), 7.37-7.85 (m, 7H, Naph—H); ¹³C NMR (75 MHz, CDCl₃) δ 13.55, 18.64,
32.29, 68.42, 116.36, 120.03, 125.44, 126.70 127.53, 127.71, 129.82, 130.90,
133.90, 148.39. MS (gc/ms) m/z (%): 336 (M⁺, 46.6), 224 (100), 115 (76.9). HRMS: m/z
calculated for C₁₈H₂₆O₄P (MH⁺) 337.1563. Found 337.1548.
Compound 36.

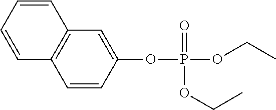 [SMILES: CCOP(=O)(OCC)Oc1ccc2ccccc2c1]

Following the general procedure described in Method C and using diethyl chlorophosphite
(10) and 2-naphthol, diethyl 2-naphthyl phosphate was obtained as a dark yellow oil: 56%
yield. ¹H NMR (400 MHz, CDCl₃) δ 1.37 (t, J = 7.0 Hz, 6H, (OCHCH₃)₂), 4.21-4.30 (m, 4H,
(OCH₂CH₃)₂), 7.26-7.84 (m, 7H, Naph—H); ¹³C NMR (100 MHz, CDCl₃) δ 16.57, 65.13,
116.76, 120.49, 125.90, 127.14 127.97, 128.14, 130.29, 131.26, 134.25, 148.76. MS
(GC/MS) m/z (%): 280.0 (M⁺, 32.9), 144.0 (89.8), 115.0 (100). HRMS: m/z calculated for
C₁₄H₁₈O₄P (MH⁺) 281.0937. Found 281.0952.
Compound 37.

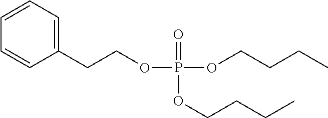 [SMILES: CCCCOP(=O)(OCCCC)OCCc1ccccc1]

Following the general procedure described in Method C and using 2-phenylethanol, di-n-
butyl 2-phenylethyl phosphate was obtained as a light yellow oil: 45% yield. ¹H NMR (400
MHz, CDCl₃) δ 0.90 (t, J = 7.4 Hz, 6H, (OCH₂CH₂CH₂CH₃)₂), 1.31-1.41 (m, 4H,
(OCH₂CH₂CH₂CH₃)₂), 1.56-1.63 (m, 4H, (OCH₂CH₂CH₂CH₃)₂), 2.98 (t, J = 7.0 Hz, 2H,
OCH₂CH₂—Ph), 3.89-3.99 (m, 4H, (OCH₂CH₂CH₂—CH₃)₂), 4.19-4.24 (q, J = 7.1 Hz, 2H,
(OCH₂CH₂—Ph), 7.20-7.31 (m, 5H, Ph—H); ¹³C NMR(100 MHz, CDCl₃) 13.56, 18.64, 32.18,
50.45, 67.59, 69.05, 127.86, 122.44, 128.55, 136.19. MS (gc/ms) m/z (%): 314 (M⁺, 0.1),
105 (100), 77 (26.5).
Compound 38.

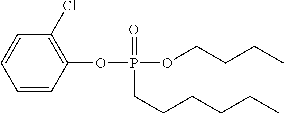 [SMILES: CCCCCCP(=O)(OCCCC)Oc1ccccc1Cl]

Following the procedure above for butyl 2-chlorophenyl ethylphosphonate and using bis(2-
chlorophenyl) hexylphosphonate, the crude product was purified by evaporative distillation
to remove 2-chlorophenol (140-165° C./0.18 mm Hg) to afford butyl 2-chlorophenyl
hexylphosphonate as an oil: 6% yield; ¹H NMR (400 MHz, CDCl₃) δ 0.89 (t, 3H, J = 6.9 Hz,
CH₃), 0.91 (t, 3H, J = 7.4 Hz, CH₃), 1.27-1.45 (m, 8H, OCH₂CH₂CH₂CH₃, (CH₂)₂(CH₂)₃CH₃),
1.60-1.77 (m, 4H, OCH₂CH₂CH₂CH₃, CH₂CH₂(CH₂)₃CH₃), 1.92-2.00 (m, 2H, CH₂(CH₂)₄CH₃),
4.05-4.21 (m, 2H, OCH₂CH₂CH₂CH₃), 7.07-7.11 (m, 1H, Ar—H),7.23 (td, 1H, J = 8.0, 1.6 Hz,
Ar—H), 7.39-7.41 (m, 1H, Ar—H), 7.45 (dt, 1H, J = 8.2, 1.4 Hz, Ar—H).
Compound 39.

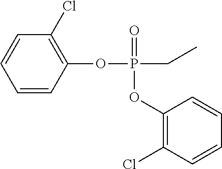 [SMILES: CCP(=O)(Oc1ccccc1Cl)Oc1ccccc1Cl]

A dried round bottom flask was charged with 0.60 mL (0.81 g, 5.5 mmol) of ethyl
phosphonic dichloride and 9 mL of dry THF. To another dried round bottom flask sodium
hydride (0.558 g of a 60% dispersion in mineral oil, approximately 14 mmol), 7 mL of dry
THF and 2.1 equivalents (1.20 mL, 11.6 mmol) of 2-chlorophenol were added. The sodium
2-chlorophenoxide was added to the stirring phosphonic dichloride solution via cannulation.
The mixture was allowed to react overnight at room temperature. The reaction mixture
was dissolved in 50 mL of diethyl ether and washed three times with 10 mL of saturated
sodium bicarbonate. The organic layer was then washed three times with 10 mL of
saturated sodium chloride solution, dried over magnesium sulfate, filtered, and
concentrated in vacuo to give 2.08 g of a pale yellow oil. The oil was purified by flash
column chromatography (silica gel, 3:7, EtOAc:hexane) and evaporatively distilled
(220° C./0.2 mm Hg) to afford bis(2-chlorophenyl) ethylphosphonate as a clear colorless oil:
28%; GCMS (m/z) 139 (100%), 295 (M⁺, 85%); R_{f} = 0.41 (2:3, hexane:EtOAc); ¹H NMR
(400 MHz, CDCl₃) δ 1.45 (dt, 3H, J = 22.0, 7.6 Hz, CH₂CH₃), 2.26 (dq, 2H, J = 18.5, 7.7
Hz, CH₂CH₃), 7.09-7.14 (m, 1H, Ar—H), 7.17-7.22 (1H, Ar—H), 7.33 (dt, 1H, J = 8.2 Hz, 1.5
Hz, Ar—H), 7.42 (ddd, 1H, J = 8.0, 1.6, 0.8 Hz, Ar—H); ¹³C NMR (100 MHz, CDCl₃) δ 19.29,
20.70, 122.26, 122.29, 125.67, 125.73, 125.95, 125.96, 127.81, 127.83, 130.57, 146.22,
146.30.
Compound 40.

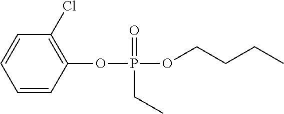 [SMILES: CCCCOP(=O)(CC)Oc1ccccc1Cl]

To a dried round bottom flask 0.420 g (1.27 mmol) of bis(2-chlorophenyl)
ethylphosphonate in 10 mL of dry THF with stirring was added. To another dried round
bottom flask sodium hydride (0.0645 g of a 60% dispersion in mineral oil, approximately
1.6 mmol) and 3 mL of dry THF were added, followed by 1.1 equivalents (0.125 mL, 1.37
mmol) of anhydrous 1-butanol. The sodium butoxide was added to the stirring phosphonate
solution via cannulation. After allowing the mixture to react for 36 hours at room
temperature, an additional 0.32 equivalents of sodium butoxide was added to the reaction
mixture via syringe. The reaction mixture was allowed to react overnight at room
temperature. The reaction mixture was diluted in 50 mL of diethyl ether and washed three
times with 10 mL of saturated sodium bicarbonate solution. The organic layer was then
washed three times with 10 mL of saturated sodium chloride solution, dried over
magnesium sulfate, filtered, and concentrated in vacuo to give 0.09 g of a yellow oil. The
crude product was purified by gravity column chromatography (gravity grade silica gel, 3:2,
EtOAc:hexane) to afford butyl 2-chlorophenyl ethylphosphonate as a clear colorless oil: 2%
yield, GCMS (m/z) 185 (100%), 276 (M⁺, 1%); ¹H NMR (400 MHz, CDCl₃) δ 0.91 (t, 3H,
J = 7.2 Hz, OCH₂CH₂CH₂CH₃), 1.24-1.43 (m, 5H, OCH₂CH₂CH₂CH₃ and PCH₂CH₃), 1.60-1.69
(m, 2H, OCH₂CH₂CH₂CH₃), 1.94-2.04 (m, 2H, PCH₂CH₃), 4.06-4.24 (m, 2H,
OCH₂(CH₂)₂CH₃), 7.10 (t, 1H, 5 Hz, Ar—H),7.21-7.26 (m, 1H, Ar—H), 7.40 (dd, 1H, J = 7.9,
1.4Hz, Ar—H), 7.46 (d, 1H, J = 12.8 Hz, Ar—H).
Compound 42.

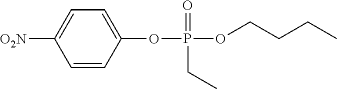 [SMILES: CCCCOP(=O)(CC)Oc1ccc([N+](=O)[O-])cc1]

To a round bottom flask with NaH (0.007 g of a 60% dispersion in mineral oil, approximately
0.18 mmol) in 1 mL dry THF was added dry 1-butanol (0.016 mL, 0.18 mmol). This solution
was added drop wise via cannulation to a solution of bis(p-nitrophenyl) ethylphosphonate
(0.093 mg, 0.26 mmol) in 3 mL dry THF in an ice/water bath. The mixture was allowed to
react at room temperature overnight. The reaction was quenched by adding 5 mL of
methylene chloride and 5 mL of NaHCO₃. The aqueous layer was extracted with methylene
chloride (3X5 mL). The organic layer was combined and dried over MgSO₄, filtered and
concentrated in vacuo. The crude product was purified by gravity column chromatography
(silica gel, 70:30, EtOAc:hexane) to give butyl p-nitrophenyl ethylphosphonate as an oil:
26% yield; Rf = 0.3 (silica gel, 70:30, EtOAc:hexane); ¹H NMR(400 MHz, CDCl₃) δ 0.92 (t,
J = 7.4 Hz, 3H, OCH₂CH₂CH₂CH₃), 1.27 (dt, J = 21.1, 7.7 Hz, 3H, PCH₂CH₃), 1.34-1.42 (m,
2H, OCH₂CH₂CH₂CH₃), 1.61-1.68 (m, 2H, OCH₂CH₂CH₂CH₃), 1.96 (dq, J = 18.3, 7.6 Hz, 2H,
PCH₂CH₃), 4.08 (dq, J = 10.1, 6.6 Hz, 1H, OCH(H)CH₂CH₂CH₃), 4.14-4.22(m, 1H,
OCH(H)CH₂CH₂CH₃), 7.39(dd, J = 9.3, 1.1 Hz, 2H, Ar—H), 8.24 (d, J = 8.9 Hz, 2H, Ar—H).
Compound 43.

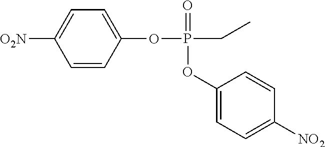 [SMILES: CCP(=O)(Oc1ccc([N+](=O)[O-])cc1)Oc1ccc([N+](=O)[O-])cc1]

To a round bottom flask charged with NaH (0.333 g, 0.0138 mol) in 5 mL dry THF was added
4-nitrophenol (1.1359 g, 0.0081 mol) in 5 mL dry THF. A solution of ethyl phosphonic
dichloride (0.6 g, 0.0040 mol) in 5mL dry THF was then added via cannulation at room
temperature. The reaction was allowed to react overnight. The reaction was quenched by
adding 15 mL of CH₂Cl₂ and 15 mL of saturated NaHCO₃. The aqueous layer was extracted
with methylene chloride 3X20 mL. The combined organic layer was dried over MgSO₄,
filtered and concentrated in vacuo. The crude solid product was dissolved in CH₂Cl₂ and the
organic layer extracted several times with saturated NaHCO₃. The organic layer was dried
over MgSO₄, filtered and concentrated in vacuo to give bis(p-nitrophenyl) ethylphosphonate
as a white solid (mp 157-159º C.): 15% yield; ¹H NMR (400 MHz, CDCl₃) δ 1.40 (dt, 3H, J =
22.4, 7.6 Hz. CH₃), 2.22 (dq, CH₂, J = 18.4, 7.6 Hz, CH₂), 7.36-7.39 (m, 2H, Ar—H), 8.23-
8.26 (m, 2H, Ar—H); ¹³C NMR (100 MHz, CDCl₃) δ 6.30, 6.38, 19.01, 20.42, 120.97, 121.02,
125.83, 144.98, 154.75, 154.84.
Compound 44.

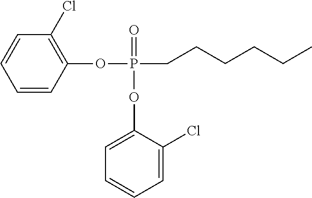 [SMILES: CCCCCCP(=O)(Oc1ccccc1Cl)Oc1ccccc1Cl]

Following the procedure above for bis(2-chlorophenyl) ethylphosphonate and using hexyl
phosphonic dichloride, the crude product was purified by evaporative distillation (160-185°
C./0.25 mm Hg) to remove 2-chlorophenol to afford bis(2-chlorophenyl) hexylphosphonate
as a clear colorless oil: 50% yield; ¹H NMR (400 MHz, CDCl₃) δ 0.88-0.91 m, 3H, CH₃),
1.30-1.35 (m, 4H, (CH₂)₃(CH₂)₂CH₃), 1.44-1.52 (m, 2H, (CH₂)₂CH₂(CH₂)₂CH₃), 1.84-1.95
(m, 2H, CH₂CH₂ (CH₂)₃CH₃), 2.18-2.27 (m, 2H, CH₂ (CH₂)₄CH₃), 7.09-7.13 (m, 1H,
Ar—H), 7.19 (td, 1H, J = 8.0, 1.7 Hz, Ar—H), 7.33 (dt, 1H, J = 8.2, 1.5 Hz, Ar—H), 7.40-7.43 (m,
1H, Ar—H).
Compound 45.

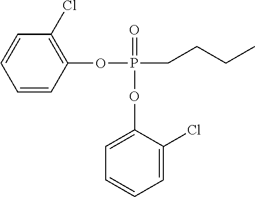 [SMILES: CCCCP(=O)(Oc1ccccc1Cl)Oc1ccccc1Cl]

Into a dried three-neck 100 mL round bottom flask equipped with an addition funnel and a
reflux condenser were added magnesium turnings (4.0 g/17 mmol) followed by 1 mL of dry
diethyl ether. To this mixture was added drop wise with stirring at room temperature a
solution of 1-bromobutane (1.8 mL/17 mmol) in 4 mL of diethyl ether over 30 minutes.
After Grignard reagent formation was complete, 2-chlorophenyl dichlorophosphate (1.23
g/5.0 mmol) in 2 mL diethyl ether was added drop wise at rt. After allowing the mixture to
stir for 2.5 h, it was worked up by addition of saturated aqueous NH₄Cl and the organic layer
separated. The aqueous layer was extracted with diethyl ether (3X) and the combined
organic layer extracted with brine, dried over MgSO₄, filtered and concentrated in vacuo.
The yellowish oil was purified by flash chromatography (silica gel, 3:2, hexane: EtOAc) to
give bis(2-chlorophenyl) butylphosphinate as an oil: 21% yield; Rf = 0.35 (3:2,
hexane: EtOAc); ¹H NMR (400 MHz, CDCl₃) δ 0.96 (t, 3H, J = 7.4 Hz, CH₂CH₂CH₂CH₃), 1.51
(sextet, 2H, J = 7.4 Hz, CH₂CH₂CH₂CH₃), 1.84-1.95 (m, 2H, CH₂CH₂CH₂CH₃), 2.19-2.28 (m,
2H, CH₂CH₂CH₂CH₃), 7.08-7.13 (m, 1H, Ar—H), 7.19 (td, 1H, J = 8.0, 1.7 Hz, Ar—H), 7.33 (dt,
1H, J = 8.2, 1.4 Hz, Ar—H), 7.41 (ddd, 1H, J = 7.9, 1.6. 0.8 Hz, Ar—H); ¹³C NMR (100MHz,
CDCl₃) δ 16.46, 16.64, 17.16, 17.21, 18.55, 19.94115.25, 115.28, 118.63, 118.69, 118.90,
118.92, 120.79, 120.80, 123.54, 139.24, 139.32.
Compound 46.

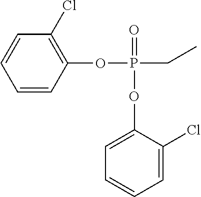 [SMILES: CCP(=O)(Oc1ccccc1Cl)Oc1ccccc1Cl]

A dried round bottom flask was charged with 0.60 mL (0.81 g, 5.5 mmol) of ethyl
phosphonic dichloride and 9 mL of dry THF. To another dried round bottom flask sodium
hydride (0.558 g of a 60% dispersion in mineral oil, approximately 14 mmol), 7 mL of dry
THF and 2.1 equivalents (1.20 mL, 11.6 mmol) of 2-chlorophenol were added. The sodium
2-chlorophenoxide was added to the stirring phosphonic dichloride solution via cannulation.
The mixture was allowed to react overnight at room temperature. The reaction mixture
was dissolved in 50 mL of diethyl ether and washed three times with 10 mL of saturated
sodium bicarbonate. The organic layer was then washed three times with 10 mL of
saturated sodium chloride solution, dried over magnesium sulfate, filtered, and
concentrated in vacuo to give 2.08 g of a pale yellow oil. The oil was purified by flash
column chromatography (silica gel, 3:7, EtOAc:hexane) and evaporatively distilled
(220° C./0.2 mm Hg) to afford bis(2-chlorophenyl) ethylphosphonate as a clear colorless oil:
28%; GCMS (m/z) 139 (100%), 295 (M⁺, 85%); R_{f} = 0.41 (2:3, hexane: EtOAc); ¹H NMR
(400 MHz, CDCl₃) δ 1.45 (dt, 3H, J = 22.0, 7.6 Hz, CH₂CH₃), 2.26 (dq, 2H, J = 18.5, 7.7
Hz, CH₂CH₃), 7.09-7.14 (m, 1H, Ar—H), 7.17-7.22 (1H, Ar—H), 7.33 (dt, 1H, J = 8.2 Hz, 1.5
Hz, Ar—H), 7.42 (ddd, 1H, J = 8.0, 1.6, 0.8 Hz, Ar—H); ¹³C NMR (100 MHz, CDCl₃) δ 19.29,
20.70, 122.26, 122.29, 125.67, 125.73, 125.95, 125.96, 127.81, 127.83, 130.57,
146.22, 146.30.
Compound 47.

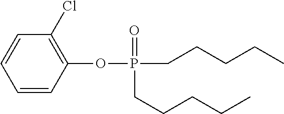 [SMILES: CCCCCP(=O)(CCCCC)Oc1ccccc1Cl]

Following the procedure above for the synthesis of phenyl dipentylphosphinate using 2-
chlorophenyl dichlorophosphate, the crude product was purified by flash chromatography
(silica gel, 3:2, hexane:EtOAc) to give 2-chlorophenyl di-n-pentylphosphinate as a golden
oil: 11% yield; R_{f} = 0.33 (silica, 3:2, hexane:EtOAc); ¹H NMR (400 MHz, CDCl₃) δ 0.89 (t,
J = 7.6 Hz, 6H, (CH₂CH₂CH₂CH₂CH₃)₂), 1.28-1.41 (m, 8H, CH₂CH₂CH₂CH₂), 1.60-1.73 (m,
4H, PCH₂CH₂), 1.86-1.93 (m, 4H, PCH₂CH₂), 7.08 (t, J = 7.7 Hz, 1H, Ar—H), 7.19-7.23 (m,
1H, Ar—H), 7.39 (d, J = 7.9 Hz, 1H, Ar—H), 7.55 (dd, J = 8.1, 1.0 Hz, 1H, Ar—H).
Compound 48.

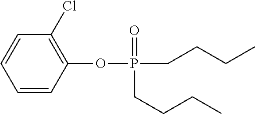 [SMILES: CCCCP(=O)(CCCC)Oc1ccccc1Cl]

The title compound was also isolated during the chromatographic purification of bis(2-
chlorophenyl) n-butylphosphonate. Therefore, the 2-chlorophenyl di-n-butylphosphinate
was further purified by flash chromatography (silica gel, 4:1 to 3:2, hexane:EtOAc) to give
the title compound as an oil: 9% yield; Rf = 0.23 (3:2, hexane:EtOAc); 0.92 (t, 6H, J = 7.2
Hz, CH₂CH₂CH₂CH₃), 1.42 (sextet, 4H, J = 7.4 Hz, CH₂CH₂CH₂CH₃), 1.54-1.75 (m, 4H,
CH₂CH₂CH₂CH₃), 1.84-1.96 (m, 4H, CH₂CH₂CH₂CH₃), 7.06-7.10 (m, 1H, Ar—H), 7.22 (td,
1H, J = 7.9, 1.7 Hz, Ar—H), 7.39 (dd, 1H, J = 8.0, 1.6 Hz, Ar—H), 7.54 (dt, 1H, J = 8.2, 1.4 Hz,
Ar—H).
Compound 49.

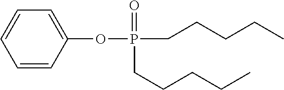 [SMILES: CCCCCP(=O)(CCCCC)Oc1ccccc1]

Into a dried three-neck 100 mL round bottom flask was added 1.5 equivalents of
magnesium turnings and 4 mL of dry THF. This mixture was stirred at room temperature.
To a second dried round bottom flask was added 1.5 equivalents of 1-bromopentane diluted
in 4 mL of THF. This mixture was stirred for five minutes and transferred to an addition
funnel. The 1-bromopentane was added to the magnesium turnings drop wise over a thirty
minute period at room temperature. The Grignard reagent begins to form fifteen minutes
after the addition was completed. To a third dried round bottom flask was added phenyl
dichlorophosphate dissolved in 1 mL of THF and transferred to the addition funnel. The
phenyl dichlorophosphate/THF was added to the 1-bromopentane mixture over a fifteen
minute period at 0° C. The reaction mixture was stirred at 0º C. for 35 minutes and then
removed from the ice bath and allowed to continue stirring for 1.5 hours at room
temperature. A golden liquid was obtained and was worked up using saturated NH₄Cl. The
organic layer was isolated and washed with brine and dried over MgSO₄, filtered,
concentrated in vacuo and purified by flash chromatography (silica gel, 3:2, hexane:EtOAc)
to give phenyl di-n-pentylphosphinate as a golden oil: 3% yield; R_{f} = 0.33 (silica, 3:2,
hexane: EtOAc); ¹H NMR (400 MHz, CDCl₃) d 0.8 (t, J = 7.2 Hz, 6H, (CH₂CH₂CH₂CH₂CH₃)₂),
1.29-1.39 (m, 8H, CH₂CH₂CH₂CH₂), 1.61-1.68 (m, 4H, PCH₂CH₂), 1.79-1.88 (m, 4H,
PCH₂CH₂), 7.12-7.34 (m, 5H, Ph—H); ¹³C NMR (100MHz, CDCl₃) d 13.76, 13.78, 21.53,
21.56, 22.11, 32.84, 32.99, 95.33, 96.08, 120.62, 120.67, 124.57, 129.50, 129.64,
129.75.
Compound 52.

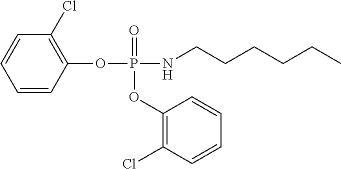 [SMILES: CCCCCCNP(=O)(Oc1ccccc1Cl)Oc1ccccc1Cl]

Into a flame-dried 50 mL round bottom flask was added 2.85 mmol of bis(2-chlorophenyl)
chlorophosphate dissolved in 4 mL of CH₂Cl₂. This mixture was stirred for fifteen minutes
at 0° C. Into a separate flame dried 50 mL round bottom flask was placed 1.6 equivalents
of n-hexylamine dissolved in 4 mL of CH₂Cl₂ and 1.9 equivalents of pyridine. This solution
was allowed to stir for ten minutes at 0° C. The hexylamine/pyridine solution was then
added to bis(2-chlorophenyl) chlorophosphate drop wise over a ten minute period via
syringe. The reaction was stirred at 0° C. for ten minutes then stirred at room temperature
overnight. Reaction mixture was diluted with 16 mL of CH₂Cl₂ and washed three times with
20 mL of 10% HCl. The organic layer was washed two times with 18 mL of saturated
NaHCO₃ solution, dried over MgSO₄, and concentrated in vacuo to give bis(2-chlorophenyl)
N-hexyl phosphoramidate as a golden oil: 71% yield; ¹H NMR (400 MHz, CDCl₃) δ 0.85 (t,
3H, J = 7.0 Hz, CH₃), 1.19-1.28(m, 6H, CH₂CH₂CH₂CH₃), 1.43-1.50 (m, 2H, NHCH₂CH₂), 3.1-
3.2 (dq, 2H, J = 10.8, 7.0 Hz, NHCH₂), 3.56 (dt, 1H, J = 13.4, 6.7 Hz, NH), 7.07-7.11 (m,
2H, Ar—H), 7.21(td, 2H, J = 7.9, J = 1.6, Ar—H), 7.40 (dt, 2H, J = 8.0, 1.3 Hz, Ar—H), 7.54 (dt,
2H, J = 8.2, 1.3 Hz, Ar—H); ¹³C NMR (100 MHz, CDCl₃) δ 13.95, 22.48, 26.06, 31.33, 31.40,
41.87, 121.74, 121.76, 125.45, 125.52, 125.66, 125.67, 127.74, 127.75, 130.46, 146.81,
146.87.
Compound 53.

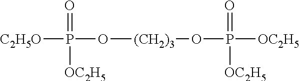

Following the general procedure described in Method D, tetraethyl propane-1,3-diyl
bisphosphate was obtained as a colorless oil: 65.0% isolated yield; ¹H-NMR (400 MHz,
CDCl₃) δ 1.34 (t, J = 8.0, 12H, (CH₃)₄), 2.02-2.08 (m, 2H, OCH₂CH₂), 4.08-4.18 (m, 12H,
(OCH₂)₆); ¹³C NMR (100 MHz, 41 CDCl₃) δ 16.22, 31.16, 63.67, 64.02. HRMS: m/z [MH]⁺
calcd for C₁₁H₂₇O₈P₂: 349.1176; found: 349.1182.
Compound 54.

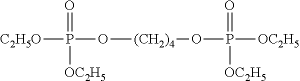

Following the general procedure described in Method D, tetraethyl butane-1,4-diyl
bisphosphate was obtained as a colorless oil: 86.5% isolated yield; ¹H NMR (400 MHz,
CDCl₃) δ 1.35 (t, J = 8.0, 12H, (CH₃)₄), 1.79-1.82 (m, 4H, (OCH₂CH₂)₂), 4.08-4.16 (m,
12H, (OCH₂)₆); ¹³C NMR (100 MHz, CDCl₃) δ 16.28, 26.66, 63.90, 67.00. HRMS: m/z [MH]⁺
calcd for C₁₂H₂₉O₈P₂: 363.1132; found: 363.1134.
Compound 55.

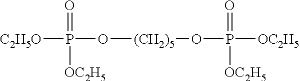

Following the general procedure described in Method D, tetraethyl pentane-1,5-diyl
bisphosphate was obtained as a colorless oil: 57.6% isolated yield; ¹H NMR (400 MHz,
CDCl₃) δ 1.34 (t, J = 8.0, 12H, (CH₃)₄), 1.47-1.53 (m, 2H, OCH₂CH₂CH₂), 1.70-1.78 (m,
4H, (OCH₂CH₂)₂), 4.01-4.15 (m, 12H, (OCH₂)₆); ¹³C NMR (100 MHz, CDCl₃) δ 16.13, 21.59,
29.78, 63.75, 67.29. HRMS: m/z [MH]⁺ calcd for C₁₃H₃₁O₈P₂: 377.1489; found: 377.1499.
Compound 56.

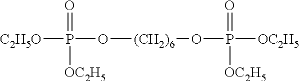

Following the general procedure described in Method D, tetraethyl hexane-1,6-diyl
bisphosphate was obtained as a colorless oil: 39.0% isolated yield; ¹H NMR (400 MHz,
CDCl₃) δ 1.34 (t, J = 8.0, 12H, (CH₃)₄), 42 1.41-1.43 (m, 4H, (OCH₂CH₂CH₂)₂), 1.66-1.72
(m, 4H, (OCH₂CH₂)₂), 4.00-4.14 (m, 12H, (OCH₂)₆); ¹³C NMR (100 MHz, CDCl₃) δ 16.18,
25.11, 30.21, 63.76, 67.50. HRMS: m/z [MH]⁺ calcd for C₁₄H₃₃O₈P₂: 391.1645; found:
391.1643.
Compound 57.

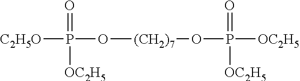

Following the general procedure described in Method D, tetraethyl heptane-1,7-diyl
bisphosphate was obtained as a colorless oil: 50% isolated yield; ¹H NMR (400 MHz, CDCl₃)
δ 1.32-1.36 (m, 18H, (CH₃)₄, OCH₂CH₂CH₂CH₂, (OCH₂CH₂CH₂)₂), 1.64-1.70 (m, 4H,
(OCH₂CH₂)₂), 4.00-4.15 (m, 12H, (OCH₂)₆); ¹³C NMR (100 MHz, CDCl₃) δ 16.21, 20.87,
25.43, 28.75, 30.26, 63.87, 67.74. HRMS: m/z [MH]⁺ calcd for C₁₅H₃₅O₈P₂: 405.1802;
found: 405.1805.
Compound 58.

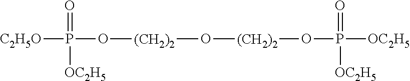

Following the general procedure described in Method D, tetraethyl 3-oxapentane-1,5-diyl
bisphosphate was obtained as a colorless oil: 6.4% isolated yield; ¹H NMR (400 MHz, CDCl₃)
δ 1.32-1.36 (t, J = 8.0, 12H, (CH₃)₄), 3.71-3.74 (m, 4H, CH₂OCH₂), 4.09-4.19 (m, 12H,
(OCH₂)₆); ¹³C NMR (100 MHz, CDCl₃) δ 16.18, 63.96, 66.48, 70.18. HRMS: m/z [MH]⁺ calcd
for C₁₂H₂₉O₉P₂: 379.1281; found: 379.1291.
Compound 59.

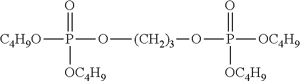

Following the general procedure described in Method D, tetrabutyl propane-1,3-diyl
bisphosphate was obtained as a colorless oil: 78% isolated yield; ¹H NMR (400 MHz, CDCl₃)
δ 0.95 (t, J = 8.0, 12H, (CH₃)₄), 1.37-1.47 (m, 8H, (CH₃CH₂)₄), 1.64-1.71 (m, 8H,
(CH₃CH₂CH₂)₄), 2.05-2.08 (m, 2H, OCH₂CH₂), 4.03-4.07 (m, 8H, (CH₃CH₂CH₂CH₂)₄), 4.13-
4.19 (m, 4H, (OCH₂)₂); ¹³C NMR (100 MHz, CDCl₃) δ 13.53, 18.65, 31.17, 32.28, 63.53,
67.58. HRMS: m/z [MH]⁺ calcd for C₁₉H₄₃O₈P₂: 461.2428; found: 461.2436.
Compound 60.

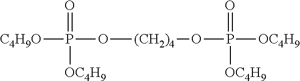

Following the general procedure described in Method D, tetrabutyl butane-1,4-diyl
bisphosphate was obtained as a colorless oil: 86.9% isolated yield; ¹H NMR (400 MHz,
CDCl₃) δ 0.94 (t, J = 8.0, 12H, (CH₃)₄), 1.36-1.46 (m, 8H, (CH₃CH₂)₄), 1.63-1.70 (m, 8H,
(CH₃CH₂CH₂)₄), 1.78-1.80 (m, 4H, (OCH₂CH₂)₂), 4.01-4.07 (m, 12H, (CH₃CH₂CH₂CH₂)₄,
(OCH₂)₂); ¹³C NMR (100 MHz, CDCl₃) δ 13.62, 18.74, 26.61, 32.38, 66.93, 67.57. HRMS:
m/z [MH]⁺ calcd for C₂₀H₄₅O₈P₂: 475.2584; found: 475.2585.
Compound 61.

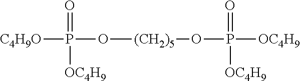

Following the general procedure described in Method D, tetrabutyl pentane-1,5-diyl
bisphosphate was obtained as a colorless oil: 91.7% isolated yield; ¹H NMR (400 MHz,
CDCl₃) δ 0.94 (t, J = 8.0, 12H, (CH₃)₄), 1.37-1.46 (m, 10H, O(CH₂)₂CH₂, (CH₃CH₂)₄), 1.63-
1.75 (m, 12H, (OCH₂CH₂)₂, (CH₃CH₂CH₂)₄), 4.01-4.06 (m, 12H, (CH₃CH₂CH₂CH₂)₄,
(OCH₂)₂); ¹³C NMR (100 MHz, CDCl₃) δ 13.63, 18.75, 21.67, 29.90, 32.38, 67.33, 67.53.
HRMS: m/z [MH]⁺ calcd for C₂₁H₄₇O₈P₂: 489.2741; found: 489.2742.
Compound 62.

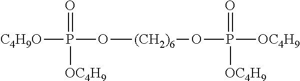

Following the general procedure described in Method D, tetrabutyl hexane-1,6-diyl
bisphosphate was obtained as a colorless oil: 42% isolated yield; ¹H NMR (400 MHz, CDCl₃)
δ 0.95 (t, J = 8.0, 12H, (CH₃)₄), 45 1.38-1.45 (m, 12H, (OCH₂CH₂CH₂)₂, (CH₃CH₂)₄), 1.65-
1.71 (m, 12H, (OCH₂—CH₂)₂ (CH₃CH₂CH₂)₄), 4.01-4.07 (m, 12H, (CH₃CH₂CH₂CH₂)₄,
(OCH₂)₂); ¹³C NMR (100 MHz, CDCl₃) δ 13.64, 18.76, 25.18, 30.29, 32.39, 67.48, 67.53.
HRMS: m/z [MH]⁺ calcd for C₂₂H₄₉O₈P₂: 503.2897; found: 503.2882.
Compound 63.

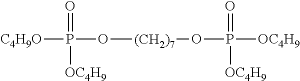

Following the general procedure described in Method D, tetrabutyl heptane-1,7-diyl
bisphosphate was obtained as a colorless oil: 66% isolated yield; ¹H NMR (400 MHz, CDCl₃)
δ 0.95 (t, J = 8.0, 12H, (CH₃)₄), 1.38-1.47 (m, 14H, (OCH₂CH₂CH₂)₂, OCH₂CH₂CH₂CH₂,
(CH₃CH₂)₄), 1.64-1.71 (m, 12H, (OCH₂CH₂)₂ (CH₃CH₂CH₂)₄), 4.01-4.07 (m, 12H,
(CH₃CH₂CH₂CH₂)₄, (OCH₂)₂); ¹³C NMR (100 MHz, CDCl₃) δ 13.62, 18.74, 25.44, 28.77,
30.27, 32.36, 67.53, 67.66. HRMS: m/z [MH]⁺ calcd for C₂₃H₅₁O₈P₂: 517.3054; found:
517.305.
Compound 64.

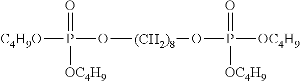

Following the general procedure described in Method D, tetrabutyl octane-1,8-diyl
bisphosphate was obtained as a colorless oil: 66% isolated yield; ¹H NMR (400 MHz, CDCl₃)
δ 0.95 (t, J = 8.0, 12H, (CH₃)₄), 1.34-1.45 (m, 16H, (OCH₂CH₂CH₂)₂, (OCH₂CH₂CH₂CH₂)₂,
(CH₃CH₂)₄), 1.63-1.69 (m, 12H, (OCH₂CH₂)₂, (CH₃CH₂CH₂)₄), 4.00-4.07 (m, 12H,
(CH₃CH₂CH₂CH₂)₄, (OCH₂)₂); ¹³C NMR (100 MHz, CDCl₃) δ 13.68, 18.79, 25.51, 29.16,
30.39, 32.43, 67.50, 67.71. HRMS: m/z [MH]⁺ calcd for C₂₄H₅₃O₈P₂: 531.3210; found:
531.3203.
Compound 65.

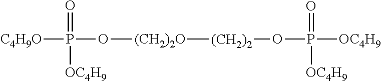

Following the general procedure described in Method D, tetrabutyl 3-oxapentane-1,5-diyl
bisphosphate was obtained as a colorless oil: 75% isolated yield; ¹H NMR (400 MHz, CDCl₃)
δ 0.94 (t, J = 8.0, 12H, (CH₃)₄), 1.37-1.46 (m, 8H, (CH₃CH₂)₄), 1.63-1.70 (m, 8H,
(CH₃CH₂CH₂)₄), 3.71-3.74 (m, 4H, (OCH₂CH₂)2O), 4.02-4.08 (m, 8H, (CH₃CH₂CH₂CH₂)₄),
4.14-4.18 (m, 4H, (OCH₂)₂); ¹³C NMR (100 MHz, CDCl₃) δ 13.58, 18.69, 32.30, 66.41,
67.63, 70.16. HRMS: m/z [MH]⁺ calcd for C₂₀H₄₅O₉P₂: 491.2533; found: 491.2521.
Compound 66.

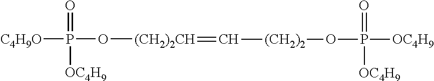

Following the general procedure described in Method D, tetrabutyl 3-hexene-1,6-diyl
bisphosphate was obtained as a colorless oil: 32% isolated yield; ¹H NMR (400 MHz, CDCl₃)
δ 0.95 (t, J = 8.0, 12H, (CH₃)₄), 1.37-1.45 (m, 8H, (CH₃CH₂)₄), 1.63-1.69 (m, 8H,
(CH₃CH₂CH₂)₄), 2.39-2.43 (m, 4H, (OCH₂CH₂)₂, 4.01-4.07 (m, 12H, (CH₃CH₂CH₂CH₂)₄,
(OCH₂)₂), 5.53-5.55 (m, 2H, CH═CH); ¹³C NMR (100 MHz, CDCl₃) δ 13.64, 18.75, 32.37,
33.71, 66.93, 67.57, 128.23. HRMS: m/z [MH]⁺ calcd for C₂₂H₄₇O₈P₂: 501.2741; found:
501.2749.
Compound 67.

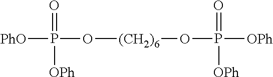

Following the general procedure described in Method D, tetraphenyl hexane-1,6-diyl
bisphosphate was obtained as a white solid: 39% isolated yield; ¹H NMR (400 MHz, CDCl₃),
1.31-1.34 (m, 4H, 47 (OCH₂CH₂CH₂)₂), 1.65-1.67 (m, 4H, (OCH₂CH₂)₂), 4.19-4.24 (m, 4H,
(OCH₂)₂), 7.16- 7.36 (m, 20H, (OC₆H₅)₄); ¹³C NMR (100 MHz, CDCl₃) δ 24.97, 30.13, 69.22,
120.19, 125.45, 129.91, 150.76. HRMS: m/z [MH]⁺ calcd for C₃₀H₃₃O₈P₂: 583.1645; found:
583.164.
Compound 68.

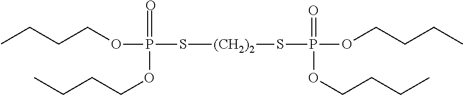

According to the representative Method E, the reaction of 1,2-dibromoethane with dibutyl
potassium thiophosphate salt followed by SiO2 flash column chromatography (60%
hexane/EtOAc) afforded O,O,O',O'-tetrabutyl-S,S'-(ethane-1,2-diyl)-Bis(phosphorothioate)
as a light yellow oil: 26%; ¹H NMR (400 MHz; CDCl₃) δ 4.09 (ddt, J = 18.4, 12.8, 6.4 Hz,
8H), 3.13 (dt, J = 16.8, 10.6 Hz, 4H), 1.72-1.65 (m, 8H), 1.46-1.37 (m, 8H), 0.94 (t, J =
7.4 Hz, 12H); ¹³C NMR (100 MHz; CDCl₃) δ 67.67 (d, J = 6.6 Hz), 32.27 (d, J = 7.3 Hz),
31.60 (t, J = 3.7 Hz), 18.86, 13.73. Anal. Calcd. for C₁₈H₄₀O₆P₂S₂: C, 45.17; H, 8.42. Found
C, 45.31; H, 8.80.
Compound 69.

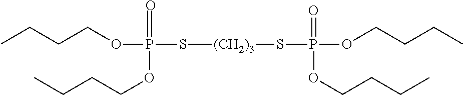

According to the representative Method G, the reaction of 1,3-propanedithiol with dibutyl
chlorophosphate followed by SiO₂ flash column chromatography (50% hexane/EtOAc)
afforded O,O,O',O'-tetrabutyl-S,S'-(propane-1,3-diyl)-Bis(phosphorothioate) as a light
yellow oil: 18%; ¹H NMR (400 MHz; CDCl₃) δ 4.08 (ddt, J = 10.0, 7.2, 6.8 Hz, 8H), 2.93
(dt, J = 15.2, 7.2 Hz, 4H), 2.09 (quintet, J = 7.1 Hz, 2H), 1.72-1.64 (m, 10H), 1.46-1.37
(m, 8H), 0.94 (t, J = 7.4 Hz, 12H); ¹³C NMR (101 MHz; CDCl₃) δ 67.52 (d, J = 6.6 Hz),
32.29 (d, J = 7.1 Hz), 31.75 (t, J = 4.9 Hz), 29.30 (d, J = 4.1 Hz), 18.88, 13.74. Anal.
Calcd for C₁₉H₄₂O₆P₂S₂: C, 46.33; H, 8.59. Found C, 45.93; H, 8.24.
Compound 70.

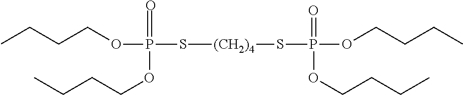

According to the representative Method G, the reaction of 1,4-butanedithiol with dibutyl
chlorophosphate followed by SiO₂ flash column chromatography (66% hexane/EtOAc)
afforded O,O,O',O'-tetrabutyl-S,S'-(butane-1,4-diyl)-Bis(phosphorothioate) as a light
yellow oil: 5%; ¹H NMR (400 MHz; CDCl₃) δ 4.09 (ddt, J = 10.2, 7.6, 6.4 Hz, 8H), 2.89-
2.80 (m, 4H), 1.83-1.78 (m, 4H), 1.72-1.65 (m, 8H), 1.46-1.40 (m, 8H), 0.94 (t, J = 7.4
Hz, 12H); ¹³C NMR (100 MHz; CDCl₃) δ 67.44 (d, J = 6.6 Hz), 32.30 (d, 39 J = 7.1 Hz),
30.30 (d, J = 4.1 Hz), 29.84 (d, J = 5.6 Hz), 18.88, 13.75; MS (GC/MS) m/z (%): 505.3
(M+, 12.6), 227.1 (14.3), 87.0 (73.1).
Compound 71.

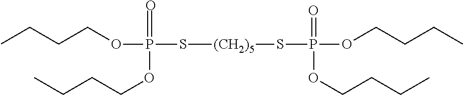

According to the representative Method G, the reaction of 1,5-pentanedithiol with dibutyl
chlorophosphate followed by SiO₂ flash column chromatography (66% hexane/EtOAc)
afforded O,O,O',O'-tetrabutyl-S,S'-(pentane-1,5-diyl)-Bis(phosphorothioate) as a light
yellow oil: 10%; ¹H NMR (400 MHz; CDCl₃) δ 4.14-4.00 (ddt, J = 10.0, 7.4, 6.4 Hz, 8H),
2.81 (dt, J = 14.3, 7.2 Hz, 4H), 1.72-1.63 (m, 12H), 1.53-1.36 (m, 10H), 0.93 (t, J = 7.4
Hz, 12H); ¹³C NMR (100 MHz; CDCl₃) δ 67.34 (d, J = 5.9 Hz), 32.20 (d, J = 7.2 Hz), 30.65,
30.34, 27.52, 18.86, 13.76; MS (GC/MS) m/z (%): 520.3 (M+, 9.0), 227.1 (12.9), 101.0
(78.1).
Compound 72.

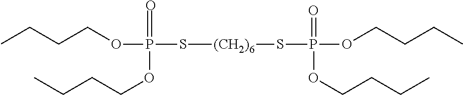

According to the representative Method E, the reaction of 1,6-dibromohexane with dibutyl
potassium thiophosphate salt followed by SiO₂ flash column chromatography (60%
hexane/EtOAc) afforded O,O,O',O'-tetrabutyl-S,S'-(hexane-1,6-diyl)-
Bis(phosphorothioate) as a light yellow oil: 37%; ¹H NMR (400 MHz; CDCl₃) δ 4.05 (ddt,
J = 10.2, 7.6, 6.4 Hz, 8H), 2.78 (dt, J = 14.3, 7.2 Hz, 4H), 1.68-1.61 (m,12H), 1.43-1.33
(m, 12H), 0.90 (t, J = 7.4 Hz, 12H); ¹³C NMR (100 MHz; CDCl₃) δ 67.30 (d, J = 6.3 Hz),
32.27 (d, J = 7.3 Hz), 30.80, 30.75 (d, J = 1.1 Hz), 28.10, 18.85, 13.72. HRMS (ESI) m/z:
[M + H]⁺ Calcd for C₂₂H₄₉O₆P₂S₂ 535.2440; Found 535.2541.
Compound 73.

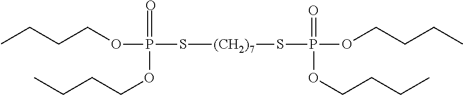

According to the representative Method E, the reaction of 1,7-dibromoheptane with dibutyl
potassium thiophosphate salt followed by SiO₂ flash column chromatography (60%
hexane/EtOAc) afforded O,O,O',O'-tetrabutyl-S,S'-(heptane-1,7-diyl)-
Bis(phosphorothioate) as a light yellow oil: 23%; ¹H NMR (400 MHz; CDCl₃) δ 4.08 (ddt,
J = 10.0, 7.6, 6.4 Hz, 8H), 2.81 (dt, J = 14.1, 7.3 Hz, 4H), 1.71-1.64 (m, 40 12H), 1.46-
1.29 (m, 14H), 0.94 (t, J = 7.4 Hz, 12H); ¹³C NMR (100 MHz; CDCl₃) δ 67.28 (d, J = 6.2
Hz), 32.27 (d, J = 7.3 Hz), 30.88 (d, J = 1.4 Hz), 30.84 (d, J = 3.5 Hz), 28.60, 28.53,
18.86, 13.72. Anal. Calcd for C₂₃H₅₀O₆P₂S₂: C, 50.35; H, 9.19. Found C, 50.25; H, 9.40.
Compound 74.

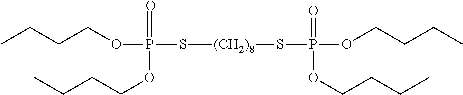

According to the representative Method F, the reaction of 1,8-octanedithiol with dibutyl
chlorophosphate followed by SiO₂ flash column chromatography (66% hexane/EtOAc)
afforded O,O,O',O'-tetrabutyl-S,S'-(octane-1,8-diyl)-Bis(phosphorothioate) as a light
yellow oil: 25%; ¹H NMR (400 MHz; CDCl₃) δ 4.07 (ddt, J = 9.8, 7.6, 6.4 Hz, 8H), 2.80 (dt,
J = 14.0, 7.3 Hz, 4H), 1.70-1.62 (m, 12H), 1.45-1.28 (m, 16H), 0.93 (t, J = 7.4 Hz, 12H);
¹³C NMR (100 MHz; CDCl₃) δ 67.27 (d, J = 6.5 Hz), 32.28 (d, J = 7.3 Hz), 30.93 (d, J =
1.5 Hz), 30.88 (d, J = 3.6 Hz), 29.00, 28.60, 18.86, 13.73. Anal. Calcd for C₂₄H₅₂O₆P₂S₂:
C, 51.22; H, 9.31. Found C, 51.53; H, 9.42.
Compound 75.

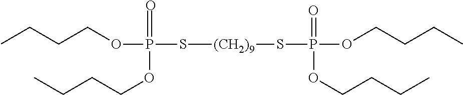

According to the representative Method E, the reaction of 1,9-dibromononane with dibutyl
potassium thiophosphate salt followed by SiO₂ flash column chromatography (66%
hexane/EtOAc) afforded O,O,O',O'-tetrabutyl-S,S'-(nonane-1,9-diyl)-
Bis(phosphorothioate) as a light yellow oil: 37%; ¹H NMR (400 MHz; CDCl₃) δ 4.08 (ddt,
J = 10.0, 7.6, 6.8 Hz, 8H), 2.81 (dt, J = 14.0, 7.4 Hz, 4H), 1.71-1.63 (m, 12H), 1.46-1.28
(m, 18H), 0.94 (t, J = 7.4 Hz, 12H); ¹³C NMR (100 MHz; CDCl₃) δ 67.28 (d, J = 6.1 Hz),
32.31 (d, J = 7.3 Hz), 30.98 (d, J = 1.5 Hz), 30.94 (d, J = 3.7 Hz), 29.45, 29.11, 28.69,
18.89, 13.76. Anal. Calcd for C₂₅H₅₄O₆P₂S₂: C, 52.06; H, 9.44. Found C, 51.82; H, 9.68.
Compound 76.

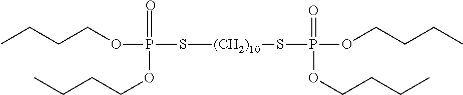

According to the representative Method E, the reaction of 1,10-dibromodecane with dibutyl
potassium thiophosphate salt followed by SiO₂ flash column chromatography (66%
hexane/EtOAc) afforded O,O,O',O'-tetrabutyl-S,S'-(decane-1,10-diyl)-
Bis(phosphorothioate) as a light yellow oil: 34%; ¹H NMR (400 MHz; 41 CDCl₃) δ 4.08 (ddt,
J = 10.0, 7.2, 6.8 Hz, 8H), 2.81 (dt, J = 14.2, 7.2 Hz, 4H), 1.71-1.63 (m, J = 14.6, 6.8
Hz, 12H), 1.46-1.27 (m, 20H), 0.93 (t, J = 7.4 Hz, 12H); ¹³C NMR (100 MHz; CDCl₃) δ
67.27 (d, J = 6.4 Hz), 32.30 (d, J = 7.3), 30.96 (t, J = 4.5 Hz), 29.52, 29.15, 28.71, 18.88,
13.75. Anal. Calcd for C₂₆H₅₆O₆P₂S₂: C, 52.86; H, 9.55. Found C, 52.56; H, 9.80.
Compound 77.

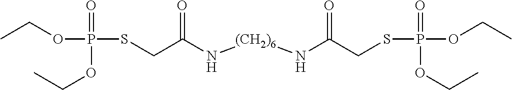

Synthesis of S',S'-(hexane-1,6-diylbis(azenediyl))-Bis(2-oxoethane-2,1-diyl)-O,O,O',O'-
Tetraethyl Diphosphorodithioate: Diethyl thiophosphate potassium salt (9 mmol), N',N'-
(hexane-1,6-diyl) bis(2-chloroacetamide) (3 mmol), 5% KI and 20 mL of dry methanol
were added to a two-neck roundbottom flask equipped with magnetic stirrer, reflux
condenser, and rubber septum under a nitrogen atmosphere. The reaction mixture was
then heated to reflux and stirred for 1-2 days while monitored periodically through TLC.
After cooling to room temperature, the solution was filtered and concentrated under
reduced pressure. After concentration, the reaction mixture was diluted with 40 mL of ethyl
acetate and washed two times with 40 mL of water. The combined aqueous layer was
extracted three times with 50 mL of ethyl acetate. The combined organic layer was washed
with 80 mL of brine and dried over MgSO₄. Evaporation of the solvent afforded a viscous
clear oil, which was purified by flash column chromatography on flash grade silica gel eluting
with ethyl acetate and ethanol (v/v) to afford the title compound: 10% yield; Rf 0.30 (10%
Ethanol/EtOAc); ¹H NMR (400 MHz; CDCl₃) δ 7.22 (t, J = 5.4 Hz, 2H), 4.15-4.01 (m, 8H),
3.35 (d, J = 18.7 Hz, 4H), 3.14 (appq, J = 6.6 Hz, 4H), 1.42 (t, J = 6.3 Hz, 4H), 1.29-1.25
(m, 16H); ¹³C NMR (100 MHz; CDCl₃) δ 168.06 (d, J = 2.4 Hz), 64.29 (d, J = 6.5 Hz),
39.57, 32.96 (d, J = 3.7 Hz), 29.03, 26.20, 15.95 (d, J = 7.2 Hz).
Compound 78.

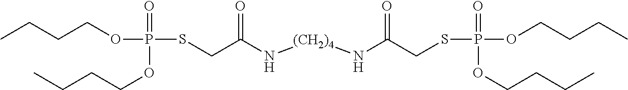

According to the representative procedure described in Method H, S',S'-(butane-1,4-
diylbis(azenediyl))-Bis(2-oxoethane-2,1-diyl)-O,O,O',O'-Tetrabutyl Diphosphorodithioate
was obtained as a viscous clear oil: 28% isolated yield; Rf 0.30 (10% Ethanol/EtOAc); ¹H
NMR (400 MHz; CDCl₃) δ 7.27 (t, J = 5.2 Hz, 2H), 4.08 (ddt, J = 6.8, 7.6, 10.0 Hz, 8H),
3.40 (d, J = 19.4 Hz, 4H), 3.24 (appq, J = 6.1 Hz, 4H), 1.70-1.63 (m, 8H), 1.57-1.53 (m,
4H), 1.46-1.37 (m, 8H), 0.92 (t, J = 7.4 Hz, 12H); ¹³C NMR (100 MHz; CDCl₃) δ 168.42
(d, J = 1.4 Hz), 68.22 (d, J = 7.2 Hz), 39.53, 32.93, 32.16, 26.63, 18.76, 13.64.
Compound 79.

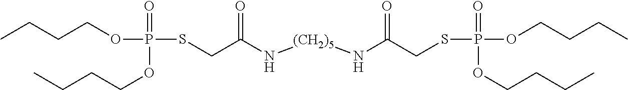

According to the representative procedure described in Method H, S',S'-(pentane-1,5-
diylbis(azenediyl))-Bis(2-oxoethane-2,1-diyl)-O,O,O',O'-Tetrabutyl Diphosphorodithioate
was obtained as a viscous clear oil: 22% isolated yield; Rf 0.30 (10% Ethanol/EtOAc); ¹H
NMR (400 MHz; CDCl₃) δ 7.20 (t, J = 5.1 Hz, 2H), 4.09 (ddt, J = 6.8, 7.6, 10.0 Hz, 8H),
3.41 (d, J = 19.6 Hz, 4H), 3.23 (appq, J = 6.4 Hz, 4H), 1.71-1.64 (m, J = 8.7, 6.4 Hz, 8H),
1.54 (quintet, J = 7.3 Hz, 4H), 1.45-1.36 (m, 10H), 0.94 (t, J = 7.4 Hz, 12H); ¹³C NMR
(100 MHz; CDCl₃) δ 168.47 (d, J = 1.5 Hz), 68.24 (d, J = 7.2 Hz), 39.71, 32.95 (d, J = 3.7
Hz), 32.20 (d, J = 7.0 Hz), 28.89, 23.99, 18.80, 13.68.
Compound 80.

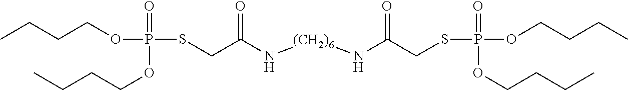

According to the representative procedure described in Method H, S',S'-(hexane-1,6-
diylbis(azenediyl))-Bis(2-oxoethane-2,1-diyl)-O,O,O',O'-Tetrabutyl Diphosphorodithioate
was obtained as a viscous clear oil: 36% isolated yield; Rf 0.30 (7% Ethanol/EtOAc); ¹H
NMR (400 MHz; CDCl₃) δ 7.19 (t, J = 5.2 Hz, 2H), 4.09 (ddt, J = 6.4, 7.6, 10.0 Hz, 8H),
3.41 (d, J = 19.7 Hz, 4H), 3.22 (appq, J = 6.5 Hz, 4H), 1.71-1.64 (m, 8H), 1.53-1.48 (m,
4H), 1.45-1.33 (m, 12H), 0.93 (t, J = 61 7.4 Hz, 12H); ¹³C NMR (100 MHz; CDCl₃) δ 168.45
(d, J = 1.3 Hz), 68.22 (d, J = 6.7 Hz), 39.79, 32.92 (d, J = 3.7 Hz), 32.18 (d, J = 7.3 Hz),
29.28, 26.45, 18.79, 13.69. HRMS (ESI) m/z:[M + H]⁺ Calcd for C₂₆H₅₅N₂O₈P₂S₂ 649.2870;
Found 649.2975.
Compound 81.

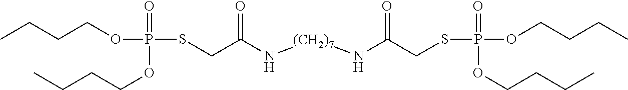

According to the representative procedure described in Method H, S',S'-(heptane-1,7-
diylbis(azenediyl))-bis(2-oxoethane-2,1-diyl)-O,O,O',O'-Tetrabutyl Diphosphorodithioate
was obtained as a viscous clear oil: 25% isolated yield; Rf 0.30 (7% Ethanol/EtOAc); ¹H
NMR (400 MHz; CDCl₃) δ 7.14 (t, J = 5.1 Hz, 2H), 4.09 (ddt, J = 6.4, 7.6, 10.0 Hz, 8H),
3.40 (d, J = 19.8 Hz, 4H), 3.22 (appq, J = 6.5 Hz, 4H), 1.71-1.64 (m, 8H), 1.52-1.32 (m,
18H), 0.94 (t, J = 7.4 Hz, 12H); ¹³C NMR (100 MHz; CDCl₃) δ 168.42 (d, J = 1.3 Hz, 1C),
68.22 (d, J = 7.2 Hz), 39.98 (), 32.92 (d, J = 3.7 Hz), 32.20 (d, J = 7.3 Hz), 29.37, 28.97,
26.85, 18.80, 13.70. HRMS (ESI) m/z: [M + H]⁺ Calcd for C₂₇H₅₇N₂O₈P₂S₂ 663.3026; Found
663.3185.
Compound 82.

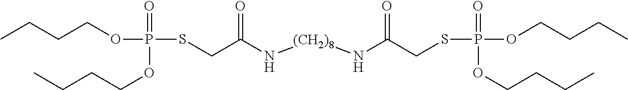

According to the representative procedure described in Method H, S',S'-(octane-1,8-
diylbis(azenediyl))-bis(2-oxoethane-2,1-diyl)-O,O,O',O'-Tetrabutyl Diphosphorodithioate
was obtained as a viscous clear oil: 14% isolated yield; Rf 0.30 (5% Ethanol/EtOAc); ¹H
NMR (400 MHz; CDCl₃) δ 7.15 (t, J = 5.0 Hz, 2H), 4.09 (ddt, J = 6.4, 7.6, 10.0 Hz, 8H),
3.40 (d, J = 19.7 Hz, 4H), 3.22 (appq, J = 6.5 Hz, 4H), 1.67 (m, 8H), 1.51-1.28 (m, 20H),
0.93 (t, J = 7.4 Hz, 12H); ¹³C NMR (100 MHz; CDCl₃) δ 168.40 (d, J = 1.3 Hz), 68.20 (d,
J = 7.2 Hz), 40.04, 32.93 (d, J = 3.7 Hz), 32.18 (d, J = 7.1 Hz), 29.39, 29.24, 26.88, 18.78,
13.68. HRMS (ESI) m/z: [M + H]⁺ Calcd for C₂₈H₅₉N₂O₈P₂S₂ 677.3183; Found 677.3310.
Compound 83.

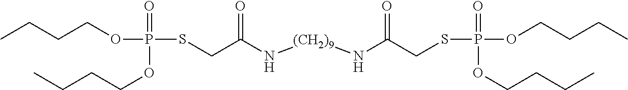

According to the representative procedure described in Method H, S',S'-(nonane-1,9-
diylbis(azenediyl))-Bis(2-oxoethane-2,1-diyl)-O,O,O',O'-Tetrabutyl Diphosphorodithioate
was obtained as a viscous clear oil: 45% isolated yield; Rf 0.20 (5% Hexane/EtOAc); ¹H
NMR (400 MHz; CDCl₃) 62 δ 7.12 (t, J = 4.9 Hz, 2H), 4.01 (ddt, J = 6.8, 7.6, 10.0 Hz, 8H),
3.40 (d, J = 19.7 Hz, 4H), 3.22 (appq, J = 6.5 Hz, 4H), 1.71-1.64 (m, 8H), 1.53-1.28 (m,
22H), 0.93 (t, J = 7.4 Hz, 12H); ¹³C NMR (100 MHz; CDCl₃) δ 168.41 (d, J = 1.3 Hz), 68.21
(d, J = 6.7 Hz), 40.07, 32.94 (d, J = 3.9 Hz), 32.19 (d, J = 6.9 Hz), 29.51, 29.43, 29.32,
26.95, 18.79, 13.70.
Compound 84.

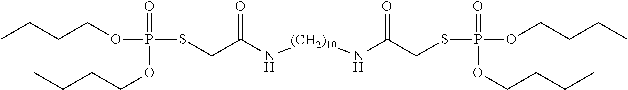

According to the representative procedure described in Method H, S',S'-(decane-1,10-
diylbis(azenediyl))-Bis(2-oxoethane-2,1-diyl)-O,O,O',O'-Tetrabutyl Diphosphorodithioate
was obtained as a viscous clear oil: 14% isolated yield; Rf 0.24 (10% Hexane/EtOAc); ¹H
NMR (400 MHz; CDCl₃) δ 7.13 (t, J = 5.0 Hz, 2H), 4.09 (ddt, J = 6.4, 7.6, 10.0 Hz, 8H),
3.40 (d, J = 19.7 Hz, 4H), 3.22 (appq, J = 6.5 Hz, 4H), 1.71-1.64 (m, 8H), 1.53-1.25 (m,
24H), 0.93 (t, J = 7.4 Hz, 12H); ¹³C NMR (100 MHz; CDCl₃) δ 168.41, 68.21 (d, J = 7.2
Hz), 40.09, 32.94 (d, J = 3.8 Hz), 32.18 (d, J = 7.3 Hz), 29.57, 29.43, 29.38, 26.99, 18.78,
13.69. HRMS (ESI) m/z: [M + H]⁺ Calcd for C₃₀H₆₃N₂O₈P₂S₂ 705.3496; Found 705.3608.
Representative Synthetic Method for Benzyl Phosphonate Derivatives (e.g., compounds 85-
92).
To a dry 50 mL round bottom flask containing a magnetic stir bar and flushed with N₂ gas,
2.70 mL (2.50 g, 10.0 mol) of tributyl phosphite and 1.06 mL (1.17 g, 9.23 mmol) of benzyl
chloride were added using syringes. The reaction mixture was heated to 170° C. under reflux
for 15 minutes. Then, the reaction mixture was allowed to stir for 2 hours under further
reflux. The temperature was monitored using a thermometer dipped in a heating mantle
filled with sand. The crude reaction mixture was analyzed by GC/MS after each 1 hr of
reaction time. After tributyl phosphite was consumed (usually after 2 hours, monitored by
GC/MS), the reaction mixture was allowed to cool to room temperature. The crude reaction
mixture was then analyzed by GC/MS and purified by column chromatography (Ethyl
acetate/hexane in 1:3 v/v), if necessary.
Compound 85, 50% yield.
Compound 87, 38% yield.
Compound 89, 85% yield.
Compound 90, 22% yield.
Compound 91, 80% yield.
Compound 92, 24% yield.
Representative procedure for compounds 93-98. To a dried two-necked, 150 mL round
bottom flask 0.71 mL (1.0 g, 4.74 mmol) of 3 dissolved in 10 mL of CH₂Cl₂ with stirring
was added. The solution was cooled to 0° C. Then 2.5 equivalents of alcohol and 2.5
equivalents of pyridine were added to the solution via syringe. The reaction was stirred
overnight at room temperature. The reaction mixture was diluted with 40 mL of diethyl
ether and washed three times with 20 mL of 10% HCl. The aqueous layers were combined
and washed with 20 mL of CH₂Cl₂. The combined organic layer was washed once with 20
mL of saturated sodium bicarbonate, once with 20 mL of brine, dried over magnesium
sulfate, filtered, concentrated in vacuo, and purified by flash chromatography or
evaporative distillation to give a clear colorless oil.
Dimethyl phenyl phosphate (93).

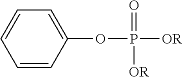

R = CH₃
Following the general procedure described above and using methanol, 1a was obtained as
a clear oil: 62.8% yield; b.p. 139° C./0.3 mm Hg; 1H NMR (400 MHz, CDCl3) d 3.83 (d,
J = 11.4 Hz, 6H, (OCH₃)₂), 7.14 (t, J = 7.4 Hz, 1H, Ph—H), 7.18 (d, J = 8.3 Hz, 2H, Ph—H),
7.31 (t, J = 7.6 Hz, 2H, Ph—H); ¹³C NMR (100 MHz, CDCl3) d 54.80, 54.87, 119.90, 119.94,
125.22, 129.85, 150.68, 150.75. Anal. (C₈H₁₁O₄P): C, H.
Diethyl phenyl phosphate (94).

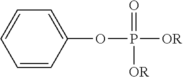

R = CH₂CH₃
Following the general procedure described above and using ethanol, 1b was obtained as a
clear oil: 38.7% yield; b.p. 130° C./0.2 mm Hg; 1H NMR (400 MHz, CDCl₃) d 1.26 (t, J =
7.2 Hz, 6H, (OCH₂CH₃)₂), 4.10-4.17 (m, 4H, (OCH₂CH₃)₂), 7.08 (t, J = 7.4 Hz, 1H, Ph—H),
7.14 (d, J = 7.86 Hz, 2H, Ph—H), 7.25 (t, J = 7.6 Hz, 2H, Ph—H); ¹³C NMR (100 MHz, CDCl₃)
d 16.04, 16.10, 64.53, 64.59, 119.98, 120.03, 125.00, 129.72, 150.81, 150.88. Anal.
(C₁₀H₁₅O₄P): C, H.
Di-n-propyl phenyl phosphate (95).

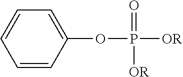

R = CH₂CH₂CH₃
Following the general procedure described above and using n-propanol, 1c was obtained as
a clear oil: 58.9% yield; b.p. 170° C./0.25 mm Hg; 1H NMR (400 MHz, CDCl₃) d 0.91 (t,
J = 7.4 Hz, 6H, (OCH₂CH₂CH₃)₂), 1.63-1.72 (m, 4H, (OCH₂CH₂CH₃)₂), 4.03-4.08 (m, 4H,
(OCH₂CH₂CH₃)₂), 7.12 (t, J = 7.4 Hz, 1H, Ph—H), 7.18 (d, J = 7.9 Hz, 2H, Ph—H), 7.29 (t, J =
7.6 Hz, 2H, Ph—H); ¹³C NMR (100 MHz, CDCl₃) d 9.96, 23.61, 23.68, 69.98, 70.05, 119.99,
120.05, 124.98, 129.69, 150.87, 150.94. Anal. (C₁₂H₁₉O₄P): C, H.
Di-n-butyl phenyl phosphate (96).

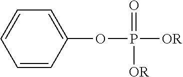

R = CH₂(CH₂)₂CH₃
Following the general procedure described above and using n-butanol, 1d was obtained as
a clear oil: 62.7% yield; b.p. 180º C./0.3 mm Hg; 1H NMR (400 MHz, CDCl₃) d 0.89 (t, J =
6.8 Hz, 6H, (OCH₂CH₂CH₂CH₃)₂), 1.37-1.55 (m, 4H, (OCH₂CH₂CH₂CH₃)₂), 1.63-1.72 (m,
4H, (OCH₂CH₂CH₂CH₃)₂), 4.03-4.08 (m, 4H, (OCH₂CH₂CH₂CH₃)₂), 7.12 (t, J = 7.4 Hz, 1H,
Ph-H), 7.18 (d, J = 7.9 Hz, 2H, Ph—H), 7.29 (t, J = 7.6 Hz, 2H, Ph—H); ¹³C NMR (100 MHz,
CDCl₃) d 13.54, 18.65, 32.22, 32.29, 68.18, 68.25, 119.99, 120.04, 124.95, 129.69,
150.88, 150.95. Anal. (C₁₄H₂₃O₄P): C, H.
Di-n-pentyl phenyl phosphate (97).

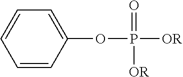

R = CH₂(CH₂)₃CH₃
Following the general procedure described above and using n-pentanol, 1e was obtained as
a clear oil: 76.5% yield; b.p. 185° C./0.25 mm Hg; 1H NMR (400 MHz, CDCl₃) d 0.92 (t, J =
7.4 Hz, 6H, (OCH₂CH₂CH₂CH₂CH₃)₂), 1.33 (s, 8H, (OCH₂CH₂CH₂CH₂CH₃)₂), 1.65-1.70 (m,
4 H, (OCH₂CH₂CH₂CH₂CH₃)₂), 4.10-4.15 (m, 4H, (OCH₂CH₂CH₂CH₂CH₃)₂), 7.15 (t, J = 7.4
Hz, 1H, Ph—H), 7.20 (d, J = 8.0 Hz, 2H, Ph—H), 7.32 (t, J = 7.6 Hz, 2H, Ph—H); ¹³C NMR
(100 MHz, CDCl₃) d 13.92, 22.20, 27.57, 29.93, 30.00, 68.46, 120.00, 120.05, 124.94,
129.67, 150.91, 150.97. Anal. (C₁₆H₂₇O₄P): C, H.
Di-cyclohexyl phenyl phosphate (98).

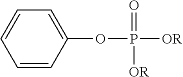

R = c-C₆H₁₁
Following the general procedure described above and using c-hexanol, 1f was obtained after
purification by flash chromatography (silica gel, 1:1, hexane:EtOAc) as a clear oil: 90%
yield; Rf (silica gel, 70:30, hexane: EtOAc) = 0.30; 1H NMR (400 MHz, CDCl₃) d 1.19-1.39
(m, 6H, cyclohexyl-H), 1.46-1.62 (m, 6H, cyclohexyl-H), 1.68-1.78 (m, 4H, (OCH(CH₂)₂)),
1.87-1.98 (m, 4H, (OCHCH₂)₂), 4.44-4.52 (m, 2H, OCH), 7.12-1.16 (m, 2H, Ph—H), 7.21-
7.24 (m, 2H, Ph—H), 7.28-7.34 (m, 1H, Ph—H); ¹³C NMR (100 MHz, CDCl₃) d 23.41, 23.44,
25.11, 33.13, 33.18, 33.27, 33.31, 78.06, 78.11, 120.08, 120.13, 124.72, 129.55, 151.00,
151.07. Anal. (C₁₆H₂₇O₄P): H; C, calcd. 63.89, found 63.47.
(±)-n-Butyl ethyl phenyl phosphate (99).

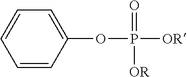

R = CH₂CH₃; R′ = CH₂(CH₂)₂CH₃
To a dried two-necked, 250 mL round bottom flask, 1.84 g (6.01 mmol) of 5 dissolved in
50 mL of dry THF was added. The solution was heated to 45° C. and 1.1 equivalents (7.21
mmol) of sodium ethoxide dissolved in 100 mL of THF was added to the stirring solution via
an addition funnel. The mixture was heated with stirring for 6 days. The reaction mixture
was partitioned between 100 mL of diethyl ether and 80 mL of saturated sodium
bicarbonate solution. The organic layer was washed twice with 40 mL of saturated sodium
bicarbonate solution. The combined aqueous layer was washed once with 80 mL of diethyl
ether. The combined organic layer was washed once with 80 mL of saturated sodium
chloride solution, dried over sodium sulfate, filtered, and concentrated vacuo to give 0.835
g of a yellow oil. The oil was purified by column chromatography (silica gel, 1:3,
EtOAc:hexane) and evaporatively distilled (169º C./0.2 mm Hg) to afford 0.45 g (29%) of
2 as a clear colorless oil: IR(neat) 3055.1 cm_1(w) 2963.7(m), 1594.5(m), 1490.2(s),
1268.8(s), 738.1(s); 1H NMR (400 MHz, CDCl₃) d 0.93 (t, J = 7.4 Hz, 3H, OCH₂CH₂CH₂CH₃),
1.34-1.43 (m, 5H, OCH₂CH₂CH₂, OCH₂CH₃), 1.64-1.69 (m, 2H, OCH₂CH₂CH₂CH₃), 4.14-
4.24 (m, 4H, OCH₂CH₂CH₂CH₃, CH₂CH₃), 7.17 (t, J = 7.2 Hz, 1H, Ph—H), 7.22 (d, J = 8.0 Hz,
2H, Ph—H), 7.34 (t, J = 7.6 Hz, 2H, Ph—H); ¹³C NMR (100 MHz, CDCl₃) d 13.53, 16.07, 16.13,
18.64, 32.21, 32.28, 64.52, 64.58, 68.20, 68.26, 119.99, 120.04, 124.98, 129.70, 150.85,
150.92. Anal. (C₁₂H₁₉O₄P): C, H.

### Compositions

In some embodiments, provided herein are compositions, comprising a compound provided herein. In some embodiments, the composition is a pharmaceutical composition, further comprising one or more pharmaceutically acceptable carriers. In some embodiments, the carrier is, a solvent or an inert stabilizer, or both. In some embodiments, the inert stabilizer provides a dehydrating effect to the composition, which may enable a longer shelf life stability of the compounds for storing the composition. In some embodiments, the compositions may further include an additional pharmaceutical agent.

Also provided herein are dosage forms suitable for administration to a mammal, comprising a compound described herein.

Thus, the compounds of the present disclosure may be formulated for oral, buccal, transdermal (e.g., patch), intranasal, parenteral (e.g., intravenous, intramuscular or subcutaneous), ophthalmic or rectal administration or in a form suitable for administration by inhalation or insufflation.

For oral administration, the compounds may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycollate); or wetting agents (e.g., sodium lauryl sulphate). The tablets may be coated by methods well known in the art. In some embodiments, the coatings are non-nutritive. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the compounds may take the form of tablets or lozenges formulated in conventional manner.

The compounds of the present disclosure may be formulated for parenteral administration by injection, including using conventional catheterization techniques or infusion. The compounds may also be formulated for topical ophthalmic administration.

Formulations for injection or topical ophthalmic administration may be presented in unit dosage form, for example in ampules, or in multi-dose containers, optionally with an added preservative. The compounds may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The compounds of the present disclosure may also be formulated in rectal compositions such as suppositories or retention enemas, e.g., containing conventional suppository bases such as cocoa butter or other glycerides.

For intranasal administration or administration by inhalation, the compounds of the present disclosure are conveniently delivered in the form of a solution or suspension from a pump spray container that is squeezed or pumped by the patient. The compounds of the disclosure can also be delivered in the form of an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the disclosure and a suitable powder base such as lactose or starch.

### Methods

The compounds described herein have esterase modulatory activity. In some embodiments, the esterase modulatory activity includes inhibition of one or more cholinesterase proteins. In some embodiments, the esterase modulatory activity includes inhibition of BCHE. In some embodiments, esterase modulation is related to diseases including inflammatory diseases, diseases associated with cholinergic dysfunction, autoimmune diseases, and long COVID, among others.

Thus, in some embodiments, the compounds described herein are useful in modulating esterase activity, e.g., cholinesterase activity, in a subject in need thereof.

In some embodiments, the compounds described herein are useful in treating an esterase- or cholinesterase-related disease, e.g., an ACHE-related or BCHE-related disease, in a subject in need thereof. In some embodiments, provided herein are methods of treating an esterase- or cholinesterase-related disease, e.g., an ACHE-related or BCHE-related disease, in a subject in need thereof, comprising administering to the subject a therapeutically effective amount of a compound described herein.

In some embodiments, the esterase-related disease includes, but is not limited to, a disease associate with the cholinergic anti-inflammatory pathway.

In some embodiments, the esterase-related disease includes, but is not limited to, an autoimmune disease.

In some embodiments, the esterase-related disease includes, but is not limited to, an inflammatory disease.

In some embodiments, the esterase-related disease includes, but is not limited to, a disease associated with cholinergic dysfunction.

In some embodiments, the esterase-related disease includes, but is not limited to, long COVID, or symptoms thereof, which may be associated with inflammation, cholinergic dysfunction, or formation of beta-amyloid plaques, or a combination thereof. In some embodiments, Long COVID disease may include inflammatory response, cholinergic dysfunction, or Alzheimer's-like symptoms.

In some embodiments, provided herein are methods of treating a disease in a subject in need thereof, comprising administering a therapeutically effective amount of a compound provided herein to the subject, wherein the disease is selected from inflammatory diseases, diseases associated with cholinergic dysfunction, autoimmune diseases, neurodegenerative disorders including Alzheimer's disease (AD), Parkinson's disease (PD), Huntington's disease (HD), and amyotrophic lateral sclerosis (ALS), or a combination thereof.

In some embodiments, provided herein are methods of treating a disease in a subject in need thereof, comprising administering a therapeutically effective amount of a compound provided herein to the subject, wherein the disease is selected from an inflammatory disease including, type 2 diabetes mellitus, hypertension, insulin resistance, hyperlipidemia, obesity, heart disease, metabolic syndrome, hyperthyroidism, nephrotic syndrome/kidney disease, proliferative diabetic retinopathy, age-related macular degeneration, chronic alcoholism, a viral infection, or a combination thereof. In some embodiments, the inflammatory disease includes type 2 diabetes mellitus, metabolic syndrome, an ophthalmic disease, or a viral infection, or a combination thereof.

Without wishing to be bound by theory, it is thought that the compounds provided herein activate the “cholinergic anti-inflammatory pathway” by way of selective inhibition of butyrylcholinesterase and associated restoration of acetylcholine to normal levels. Specifically, the compounds provided herein may be useful in a method of treatment for disease associated with cholinergic dysfunction, which in some embodiments are diseases associated with reduced acetylcholine activity and/or elevated butyrylcholinesterase levels. In some embodiments, the disease associated with cholinergic dysfunction is selected from Alzheimer's disease, dementia with Lewy Bodies, myasthenia gravis, multiple sclerosis (MS), autism spectrum disorder (ASD), Parkinson's disease, Huntington's disease, Lambert-Eaton myasthenic syndrome (LEMS), subcortical vascular dementia, opioid addiction, opiate use disorder, bipolar disorder, schizophrenia, metabolic syndrome, or glaucoma, among other cholinergic disorders. In some embodiments, the disease associated with cholinergic dysfunction includes dementia with Lewy Bodies, myasthenia gravis, multiple sclerosis (MS), autism spectrum disorder (ASD), opioid addiction, bipolar disorder, schizophrenia, an autoimmune disorder, or a combination thereof.

In some embodiments, the subject includes a genetic predisposition to increased risk of having AD or increased prevalence of beta-amyloid peptides or formation of beta-amyloid plaques, including APOE e4 Gene, other Late-Onset Alzheimer's Genes (ABCA7, CLU, CR1, PICALM, PLD3, TREM2, and SORL1) Young-Onset Alzheimer's, Family History and Genetics Mutations, genetic mutations of amyloid precursor protein (APP), Presenilin 1 (PSEN1), Presenilin 2 (PSEN2), and Down Syndrome Mutations. In some embodiments, the genetic predisposition includes APOE e4 gene, Young-Onset Alzheimer's, or Down Syndrome, or a combination thereof.

In some embodiments, the compounds provided herein are useful in various biochemical, pharmacological, or cell biology applications to study the role of BCHE in normal cell growth and development, e.g., stem cell differentiation.

In some embodiments, the subject comprises a refractory disease. In some embodiments, the refractory disease comprises a refractory esterase-related disease.

Another aspect of the present disclosure is drawn to therapeutic compositions comprising the presently disclosed compounds and a pharmaceutically acceptable carrier. The carrier may be any solid, semi-solid, or liquid material that acts as an excipient or vehicle for the active compound. The formulations may also include wetting agents, emulsifying agents, preserving agents, sweetening agents, bulking agents, coatings and/or flavoring agents. In some embodiments, the coatings can be a varnish. If used in an ophthalmic or infusion format, the formulation will usually contain one or more salts to adjust the osmotic pressure of the formulation.

The diseases or conditions which can be treated by the presently disclosed compounds include neurodegenerative diseases including, but not limited to, Alzheimer's disease.

Some embodiments include a method of treating Alzheimer's disease, comprising administering a compound described herein, to a mammal in need thereof.

Some embodiments include a method of reducing the level of a β-amyloid peptide in a brain of a mammal comprising administering an effective amount of a compound described herein, to the mammal. In some embodiments, the β-amyloid peptide is Aβ40. For example, the level of Aβ40 in the brain of the mammal can be reduced by at least about 10%, at least about 20%, or at least about 30%. In some embodiments, the β-amyloid peptide is Aβ42. For example, the level of Aβ42 in the brain of the mammal can be reduced by at least about 10%, at least about 20%, or at least about 30%.

For any method described herein, such as treating Alzheimer's disease in a mammal, or reducing the level of a β-amyloid peptide in a brain of a mammal, the mammal can be a human being that is at least about 50 years of age, at least about 65 years of age, at least about 70 years of age, at least about 75 years of age, at least about 80 years of age, or at least about 85 years of age.

The present disclosure also provides for methods of treating neurodegenerative diseases with a therapeutically effective amount of a compound disclosed herein.

Also provided herein are methods of inhibiting the formation of β-amyloid peptide in a mammal, comprising administering to the mammal a therapeutically effective amount of a compound described herein.

Also provided herein are methods of inhibiting the aggregation of β-amyloid peptide into neurotoxic plaques in a mammal, comprising administering to the mammal a therapeutically effective amount of a compound described herein.

Also provided herein are methods of treating Alzheimer's disease, comprising administering a compound described herein to a mammal in need thereof.

Also provided herein are uses of a compound described herein in the manufacture of a medicament for the treatment of a disease referred to herein.

The actual route of administration of a compound, a composition, or a combination disclosed herein used can be determined by a person of ordinary skill in the art by taking into account factors, including, without limitation, the type of esterase- or cholinesterase-related disease, the location of the esterase- or cholinesterase-related disease, the cause of the esterase- or cholinesterase-related disease, the severity of the esterase- or cholinesterase-related disease, the duration of treatment desired, the degree of relief desired, the duration of relief desired, the particular compound, composition, or combination, the rate of excretion of the compound, composition, or combination used, the pharmacodynamics of the compound, composition, or combination used, the nature of the other compounds to be included in the composition or combination, the particular route of administration, the particular characteristics, history and risk factors of the individual, such as, e.g., age, weight, general health and the like, the response of the individual to the treatment, or any combination thereof. An effective dosage amount of a compound, a composition, or a combination disclosed herein may thus be determined by the person of ordinary skill in the art considering all criteria and utilizing his best judgment on the individual's behalf.

In some embodiments, a compound described herein, is administered orally in an effective amount. For example, in some embodiments, an effective amount in a dosage form can be at least about 0.1 mg, at least about 1 mg, at least about 2 mg, at least about 5 mg, or at least about 10 mg, up to about 100 mg, up to about 200 mg, up to about 300 mg, up to about 400 mg, or up to about 500 mg, of the compound.

The potential as a therapeutic for Alzheimer's disease, or other conditions, may be improved if the compound in question is able to cross the blood brain barrier. In some embodiments, a sufficient amount of a compound described herein, is administered so that at least about 10 ng, at least about 50 ng, at least about 100 ng, up to about 1 μg, up to about 1 mg, or up to about 100 mg, of the compound crosses the blood brain barrier.

In some embodiments, sufficient amount of a compound described herein, is administered so that the compound has a concentration of at least about 10⁻¹⁰ M, at least about 10⁻⁹ M, at least about 10⁻⁸ M, up to about 10⁻⁶ M, or up to about 10⁻⁵ M, in the brain.

Aerosol formulations for treatment of the conditions referred to above in the average adult human are preferably arranged so that each metered dose or “puff” of aerosol contains 20 μg to 1,000 μg of the compound of the disclosure. The overall daily dose with an aerosol will be within the range a 100 μg to 10 mg. Administration may be several times daily, for example 2, 3, 4 or 8 times, giving for example, 1, 2 or 3 doses each time.

### Kits

In some embodiments, provided herein are packaged compounds, packaged compositions, or packaged pharmaceutical compositions, comprising a container holding a therapeutically effective amount of a compound described herein, and instructions for using the compound in accordance with one or more of the methods provided herein.

The present compounds and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (e.g. Irgacure 184, 2959), preferably water-soluble initiators (e.g. Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (e.g. by the addition of specific product information leaflets) into suitable containers (boxes, etc.).

According to further embodiments, the present compounds can also be provided in kit form combined with other components necessary for administration of the material to the patient. For example, disclosed kits, such as for use in the treatment of cancer, can further comprise, for example, administration materials.

The kits are designed in various forms based on the specific deficiencies they are designed to treat.

The compounds or compositions provided herein may be prepared and placed in a container for storage at ambient or elevated temperature. When the compound or composition is stored in a polyolefin plastic container as compared to a polyvinyl chloride plastic container, discoloration of the compound or composition may be reduced, whether dissolved or suspended in a liquid composition (e.g., an aqueous or organic liquid solution), or as a solid. Without wishing to be bound by theory, the container may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). Some containers also include the capacity to reduce adherence or adsorption of the active agent to the surface of the container. Some containers also include the capacity to reduce exposure of the container's contents to infrared light, or a second component with such a capacity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper, cardboard, paperboard, metallic film, or foil, or a combination thereof, container to further reduce exposure of the container's contents to UV, visible, or infrared light. Compounds and compositions benefiting from reduced discoloration, decomposition, or both during storage, include eye drop solutions or implants that include a compound or composition thereof provided herein. The compounds or compositions provided herein may need storage lasting up to, or longer than, three months; in some cases up to, or longer than one year. The containers may be in any form suitable to contain the contents; for example, a bag, a bottle, or a box.

The following examples further illustrate aspects of the present disclosure. However, they are in no way a limitation of the teachings or disclosure as described herein.

### EXAMPLES

### Example 1: Effects of Compounds on Cells

Porcine umbilical stem cells are cultured in Neurobasal Medium supplemented with B27 (Invitrogen) and antibiotics for various periods of with varying concentrations of compounds disclosed herein. Cells are collected, washed, counted, and analyzed for viability by Trypan Blue staining.

In addition, uptake of compounds by porcine umbilical stem cells, human umbilical cord cells, and neurons are measured after 24 and 48 hours of culture.

### Example 2: Brain Permeability

An animal study using two groups (n=7/group) of male Long-Evans rats (200-225 g, Charles River, Portage, MI) is conducted to determine whether the compounds disclosed herein cross the blood brain barrier. Rats are injected intraperitoneally with either 0.2 mL vehicle control or 0.2 mL of a 10 mg/mL solution of compound in the appropriate solvent. Thirty minutes after treatment, animals are deeply anesthetized with isoflurane and then decapitated. Brains are removed quickly, rinsed in ice cold phosphate buffer saline (pH 7.4) and block dissected and flash frozen in liquid nitrogen and stored at −80° C. until assayed.

The tissue from animals injected with the compounds is processed as follows: i) brain tissue (1.5 g) is allowed to thaw on ice in a Dounce homogenizer; ii) 10 mL of methylene chloride is added and the tissue extracted with 20 strokes of the loose fitting pestle. The homogenate is clarified by centrifugation and the supernatant is extracted three times with methylene chloride using a separatory funnel. The organic phase is collected, pooled, and dried over magnesium sulfate. The eluate is then concentrated down to a volume of approximately 200 μl under nitrogen and analyzed by GC/MS for the presence of the compound. A solution of neat compound serves as control.

### Example 3: Inhibition of β-Amyloid Peptides

One of the more significant factors affecting patients with Alzheimer's disease is the elevated level of neurotoxic β-amyloid peptide. The most prominent β-amyloid peptides are referred to as Aβ40 and Aβ42 peptides. For in vitro studies neuroblastoma cells were used as a biological model for evaluating β-amyloid peptide production. Cells are cultured in Eagles MEM containing Glutamax, penicillin and streptomycin, and 0.5% fetal calf serum. To monitor the effects of the compounds, cells are grown for 24 hrs in the presence or absence of compound. The supernatant is collected and concentrated using a 2K centrifugal membrane filter. The concentrated solution is then analyzed by ELISA for Aβ40 and Aβ42 peptides. The results are expressed in μg/mL, are shown below.

### Example 4: Effect of Compounds on Aggregation of β-Amyloid Peptides

Neurotoxic plaques, i.e., aggregates of β-amyloid peptide, bind thioflavin T (ThT) and show significant fluorescence. In contrast, the individual peptide molecules do not bind ThT and therefore do not show any fluorescence.

Aβ (1-40) peptide was solubilized in hexafluoroisopropanol (HFIP). A solution of 25 μM ThT was prepared in 0.025 M phosphate buffer, pH 6.0. Peptide was dissolved in 0.01 M PBS; 0.1M NaCl, pH7.4 to a final concentration of 30 μM. Inhibitors were prepared in methanol. The final concentration of HFIP and methanol were 2% and 10% (v/v) respectively. The control sample consisted peptide incubated in PBS/2% HFI/10% methanol without inhibitor. All samples were mixed and incubated at 25° C. for 2 hours. Fluorescence was measured by mixing 470 μL of ThT solution with 30 μL of the protein solution. Excitation and emission wavelengths were 440 nm with 5 nm slit width and 485 nm with 10 nm slit width, respectively. Biological activity was calculated as percent of inhibitory activity of inhibitor at varying concentration was calculated using the following equation: Vi=100−[(Fi−Fb)/F0]×100. The higher the Vi, the less aggregation of amyloid peptide.

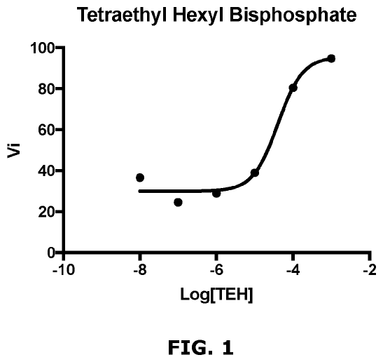

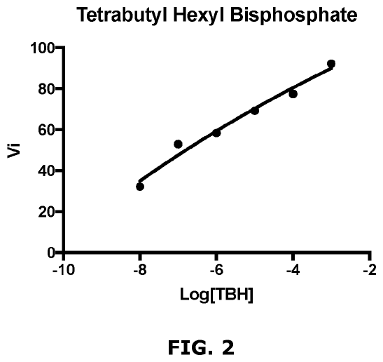

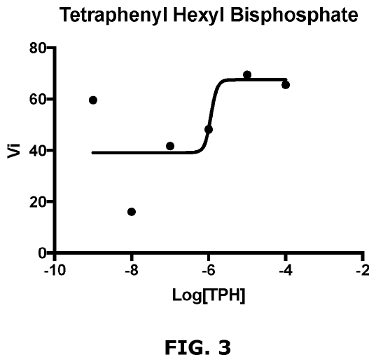

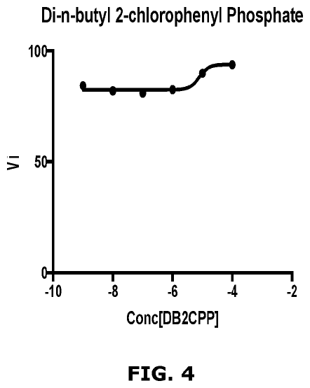

Exemplary compounds, tetraethyl hexyl biphosphate (FIG. 1), tetrabutyl hexyl bisphosphonate (FIG. 2), and tetraphenyl hexyl biphosphonate (FIG. 3) significantly inhibited aggregation of β-amyloid peptides, while di-n-butyl 2-chlorophenyl phosphate did not inhibit amyloid peptide aggregation (FIG. 4).

### Example 5: BCHE/ACHE Activity with Di-n-butyl 2-chlorophenyl phosphate (DB2CIPP)

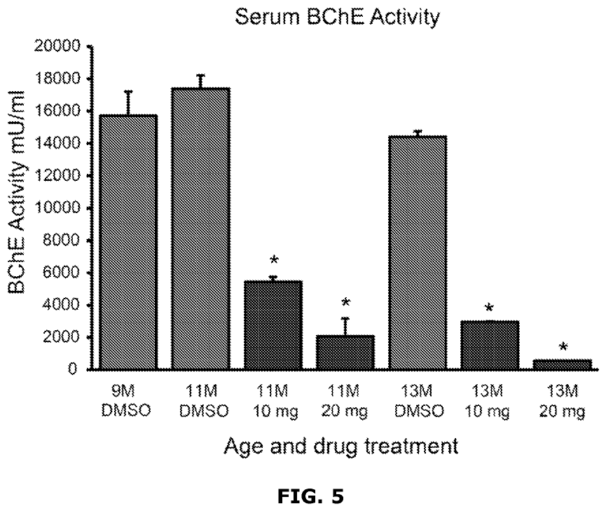

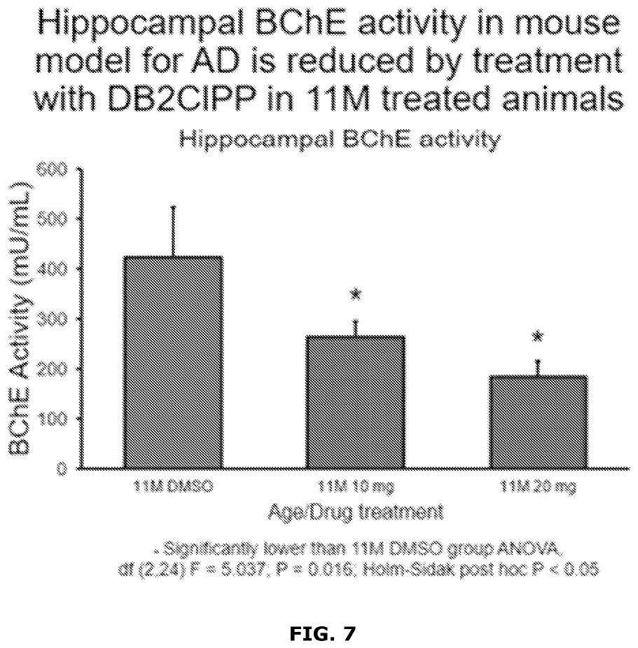

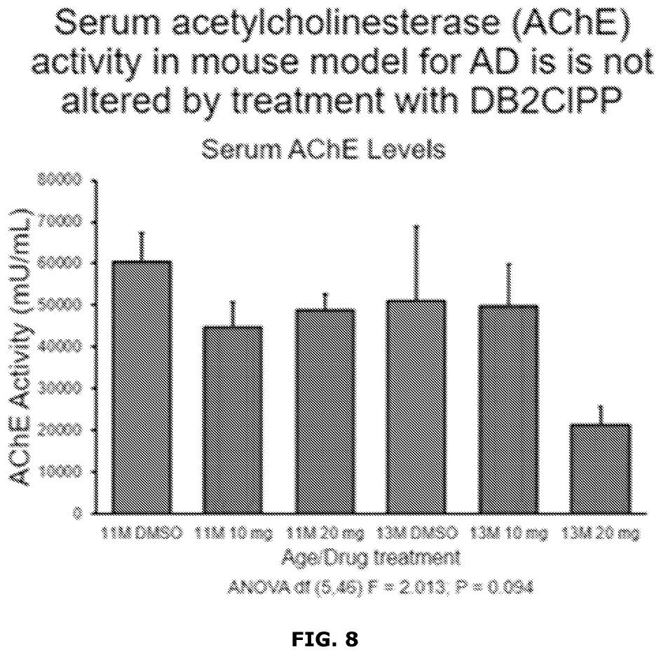

DB2CIPP Testing Results Inhibition of BChE in brain and serum. Di-n-butyl 2 chlorophenyl phosphate (DB2CIPP) inhibits BChE in brain and serum without inhibiting AChE in serum (see FIG. 5, FIG. 7, and FIG. 8). In FIG. 5, in vivo testing results of DB2CIPP show significant inhibition of serum butyrylcholinesterase activity in transgenic Alzheimer's mice using 10 mg and 20 mg doses of DB2CIPP over 11 months and 13 months. Other in vivo tests also show the ability of DB2CIPP to cross the blood brain barrier (BBB) and inhibit brain butyrylcholinesterase activity. In FIG. 7, in vivo testing results of DB2CIPP show significant inhibition of hippocampal butyrylcholinesterase activity in transgenic Alzheimer's mice using 10 mg and 20 mg doses of DB2CIPP over 11 months. In FIG. 8, in vivo testing results of DB2CIPP show serum acetylcholinesterase activity in transgenic Alzheimer's mice using 10 mg and 20 mg doses of DB2CIPP over 11 months and 13 months is not significantly altered.

### Example 6: β-Amyloid Plaque Inhibition with DB2CIPP

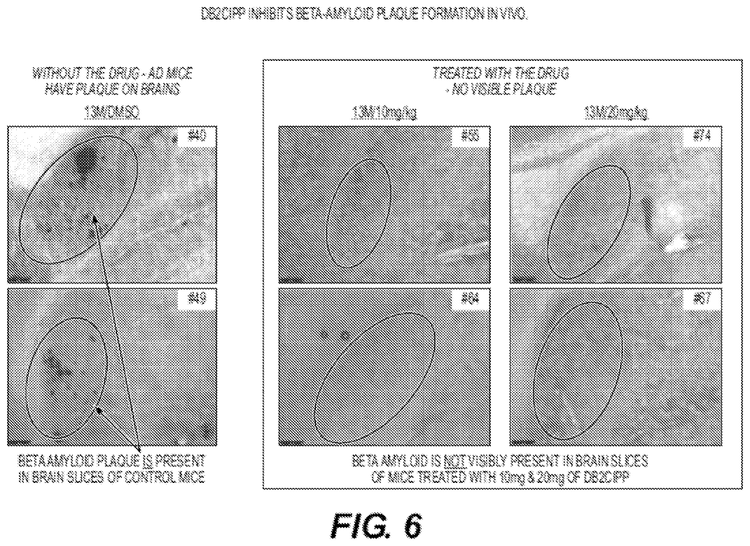

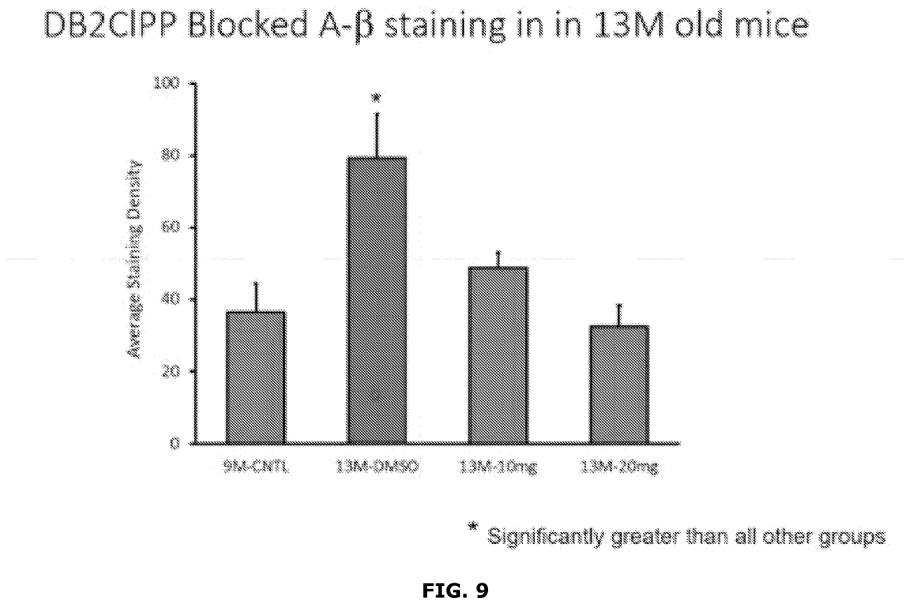

In FIG. 6, in vivo testing results of DB2CIPP show significant inhibition of 3-amyloid plaque formation in transgenic Alzheimer's mice. Images show immunohistochemistry of 13-month transgenic Alzheimer's disease mice brains at subiculum region of hippocampus, treated with DB2CIPP and DMSO control beginning at 9 months. Evidence shows that inflammation and elevated butyrylcholinesterase is associated with the pathogenesis of Alzheimer's disease and dementia, as a resulting effect on increasing amyloid-β precursor protein (APP) expression, cleavage and elevated population and/or reduced clearance of amyloid-β peptides, Aβ40 and Aβ42, and the extracellular aggregates of amyloid β (Aβ) plaques and intracellular neurofibrillary tangles made of hyperphosphorylated tau protein in cortical and limbic areas of the human brain. Selective inhibition of butyrylcholinesterase in the brain is widely documented as a potential treatment for Alzheimer's disease and dementia, by improving cognition and modulating neuropathological markers of the disease. As an inhibitor of butyrylcholinesterase, DB2CIPP may also be used to inhibit or slow the progression of the Alzheimer's-like symptoms seen in long COVID. FIG. 9 shows DB2CIPP reduced beta amyloid plaques as seen by immunopositive staining in the hippocampus compared to untreated transgenic Alzheimer's mice using 10 mg or 20 mg doses of Di-n-butyl 2-chlorophenyl phosphate (DB2CIPP) over 13 months.

### Example 7: BCHE or ACHE Inhibition Study

The compounds referred to herein have BCHE inhibitory activity. The BCHE IC₅₀ or Kᵢ for certain of the compounds referred to herein is shown in Table 3. To determine the inhibitory values, enzymes were incubated for 2 hours in the presence of varying concentrations of compound prior to activity measurements. Activity is then measured colorimetrically upon addition of substrate and Ellman's reagent.

TABLE 3

| | | BCHE | | ACHE | |
|---|---|---|---|---|---|
| | BCHE | Kᵢ ± | ACHE | Kᵢ ± | |
| | IC₅₀ | SEM | IC₅₀ | SEM | Mode of |
| Compound | (μM) | (μM) | (μM) | (μM) | Inhibition |
| 9 | 0.00069 | | 47 | | Irreversible^{g} |
| 27 | | 1.0 ± | | | Reversible |
| | | 0.4 | | | |
| 35 | | 1.9 ± | | | Reversible |
| | | 0.4 | | | |
| 53 | 8.26 | | | | |
| 54 | 8.20 | | | | |
| 55 | 35.4 | | | | |
| 56 | | 228 ± | | | Reversible |
| | | 19 | | | |
| 57 | 40.1 | | | | |
| 58 | 11.1 | | | | |
| 59 | 0.79 | | | | |
| 60 | 88.1 | | | | |
| 61 | 2.72 | | | | |
| 62 | | 4 ± | | | Reversible |
| | | 1 | | | |
| 63 | 3.95 | | | | |
| 64 | 2.39 | | | | |
| 65 | 31.2 | | | | |
| 66 | 1.20 | | | | |
| 67 | | | | | Reversible |
| 69 | | 0.21 ± | | | |
| | | 0.02 | | | |
| 71 | | 0.24 ± | | | |
| | | 0.01 | | | |
| 72 | | 0.25 ± | | | |
| | | 0.01 | | | |
| 73 | | 0.34 ± | | | |
| | | 0.05 | | | |
| 80 | | 0.067 ± | | | |
| | | 0.001 | | | |
| 81 | | 0.1 ± | | | |
| | | 0.02 | | | |
| 82 | | 0.14 ± | | | |
| | | 0.03 | | | |
| 85 | 0.08 (mM) | | | | |
| 89 | | | | | Irreversible^{g} |
| 90 | | | | | Irreversible^{g} |
| 91 | 0.066 (mM) | | | | |
| 92 | | | | | Irreversible^{g} |
| 93 | | 1.7 ± | | 6.5 ± | |
| | | 0.3 | | 0.8 | |
| | | (mM) | | (mM) | |
| 94 | | 0.055 ± | | 3.1 ± | |
| | | | | 0.7 | |
| | | 0.008 | | (mM) | |
| | | (mM) | | | |
| 95 | | 0.08 ± | | ND^{b} | |
| | | 0.02 | | | |
| | | (mM) | | | |
| 96 | | 0.043 ± | | ND^{c} | |
| | | 0.007 | | | |
| | | (mM) | | | |
| 97 | | 0.006 ± | | ND^{d} | |
| | | 0.002^{a} | | | |
| | | (mM) | | | |
| 98 | | 0.007 ± | | ND^{e} | |
| | | 0.001^{a} | | | |
| | | (mM) | | | |
| 99 | | 10 ± | | ND^{f} | |
| | | 0.03 | | | |
| | | (mM) | | | |

^{a}Partial competitive.
^{b}<20% Inhibition at [lc] = 5 mM and [ATC] = 0.1 mM.
^{c}No inhibition at [1d] = 1 mM and [ATC] = 0.1 mM.
^{d}<8% Inhibition at [le] = 3 mM and [ATC] = 0.1 mM.
^{e}No inhibition at [1f] = 3 mM and [ATC] = 0.1 mM.
^{f}<8% inhibition at [2] = 3 mM and [ATC] = 0.1 mM.
^{g}enzyme covalently modified.

### Example 8: Relative Activity of BCHE and ACHE

In order to establish the inhibitor's differential inhibitory ability against the two enzymes (see Tables 4 and 5), a relative enzyme inhibitory value (“Relative Activity”) was obtained. The relative activities were measured by treating each enzyme with either an overwhelming amount of the inhibitor (50-1000 μM) or none, and establishing the enzyme activity under the two conditions. Table 4 lists the Relative Activity (=Activity of Inhibitor Incubated enzyme/Activity of unincubated enzyme) against BCHE and ACHE.

For example, if the enzyme activity was unchanged under the two conditions, then the relative activity would be (nearly) one and the inhibitor was deemed to be a very poor inhibitor of the enzyme. This was observed for ACHE when treated with inhibitors 56 and 61. Meanwhile, enzyme activity decreased significantly when BCHE was exposed to excess amounts of inhibitors 56 and 61, with 61 showing a higher inhibition of BCHE than 56.

TABLE 4
Relative Activity of BCHE and ACHE

| Compound | BCHE Activity Ratio* | ACHE Activity Ratio* |
|---|---|---|
| 56 | 0.33 ± 0.03 | 0.93 ± 0.03 |
| 61 | 0.10 ± 0.00 | 0.97 ± 0.03 |

In order to compare the relative inhibitory activity of the five compounds 27, 35, 89, 90 and 92 against BCHE and ACHE, a solution of each enzyme was incubated with 20 μM of the inhibitor for 2 minutes, and the enzyme activity determined. Table 5 lists the Relative Activity (=Activity of Inhibitor incubated enzyme/Activity of unincubated enzyme).

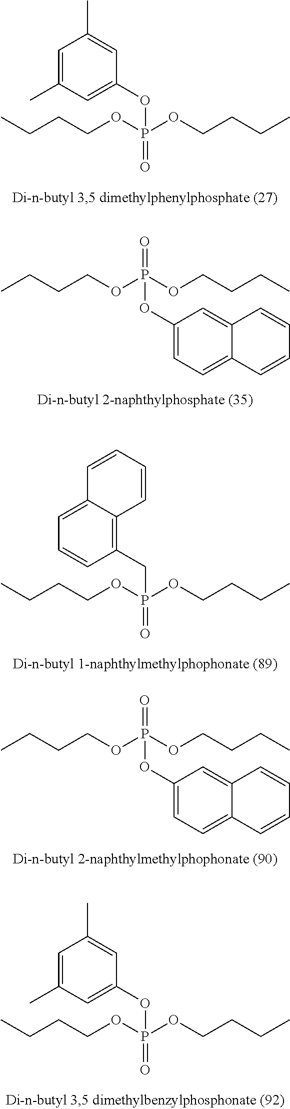

TABLE 5

| Compound | Relative Activity of BCHE | Relative Activity of ACHE |
|---|---|---|
| 27 | 0.45 | 0.93 |
| 35 | 0.10 | 0.84 |
| 89 | 0.47 | 0.96 |
| 90 | 0.45 | 0.95 |
| 92 | 0.64 | 0.99 |

### Example 9: Tau

To determine whether a compound herein inhibits Tau protein formation, transgenic mouse models (Taconic Alzheimer Model 2508 or triple transgenic mouse models with early onset overexpression of tau (Model Trem2^{R47H NSS} from the UCI MODEL-AD Group)) are treated with test compound, e.g., di-n-butyl 2-naphthylmethylphosphonate (dibutyl (naphthalen-2-ylmethyl)phosphonate), di-n-butyl 3,5-dimethylbenzylphosphonate (dibutyl (3,5-dimethylbenzyl)phosphonate), or di-n-butyl 2-chlorophenyl phosphate, or control vehicle. The treatments are started just prior to the onset of tau formation in the brain and continue for three months. Levels of Tau expression are measured by western blot and immunohistochemistry in the brain. Comparisons are made among animals that received a test compound and control injections as well as tissue collected from animals at the time treatments are initiated. It is found that mice treated with test compound have less Tau expression than control treated groups. Further, test compound treated animals are similar to the tissue harvested from mice at the onset of treatment.

### Example 10: Inflammation

To determine whether a compound herein reduces inflammation, inflammatory indicators are measured in the animals treated for Tau expression in Example 9. Levels of AD-related cytokines are measured using a kit that screens for a panel of inflammatory cytokines and chemokines which include but not limited to interleukin-1 (IL-1), tumor necrosis factor-alpha (TNF-alpha), interleukin-6 (IL-6), interleukin-10 (IL-10), transforming growth factor-beta (TGF-beta). Infiltration of activated microglia is also measured as a marker of neuroinflammation in the brain regions of interest. It is found that test compound reduces inflammatory indicators and activated microglial infiltration compared to untreated mice and the tissue harvested from mice at the start of treatment.

While the disclosure has been described in detail and with reference to specific embodiments thereof, it will be apparent to one skilled in the art that various changes and modifications can be made without departing from the spirit and scope of the disclosure.

Groupings of alternative elements or embodiments disclosed herein may be referred to and claimed individually or in any combination with other members of the group or other elements found herein.

The contents of all references (including literature references, issued patents, published patent applications, and co-pending patent applications) cited throughout this application are hereby expressly incorporated herein in their entireties. Unless otherwise defined, all technical and scientific terms used herein are accorded the meaning commonly known to one with ordinary skill in the art.

Those skilled in the art will recognize, or be able to ascertain, using no more than routine experimentation, many equivalents of the specific embodiments of the invention described herein. Such equivalents are intended to be encompassed by the following claims.

We claim:

1. A compound, which is

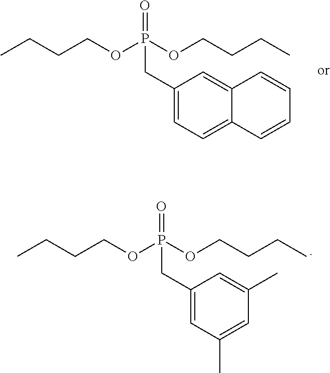 [SMILES: CCCCOP(=O)(Cc1ccc2ccccc2c1)OCCCC ; CCCCOP(=O)(Cc1cc(C)cc(C)c1)OCCCC]

2. A composition, comprising the compound of claim 1.

3. The composition of claim 2, which is a pharmaceutical composition further comprising a pharmaceutically acceptable carrier.

4. A method of treating an esterase-related disease, e.g., a cholinesterase-related disease, e.g., an ACHE-related or BCHE-related disease, in a subject in need thereof, comprising administering to the subject a therapeutically effective amount of the compound of claim 1 or the composition of claim 2 or claim 3.

5. The method of claim 4, wherein the esterase-related disease includes a disease associate with the cholinergic anti-inflammatory pathway.

6. The method of claim 4, wherein the esterase-related disease includes an autoimmune disease.

7. The method of claim 4, wherein the esterase-related disease includes an inflammatory disease.

8. The method of claim 4, wherein the esterase-related disease includes a disease associated with cholinergic dysfunction, and wherein by treating the disease associated with cholinergic dysfunction, the symptomatic effects of ACh deficiency are treated.

9. The method of claim 4, wherein the esterase-related disease includes, but is not limited to, long COVID disease, or symptoms thereof, which may be associated with inflammation, cholinergic dysfunction, or formation of beta-amyloid plaques, or a combination thereof, and wherein by treating the disease associated with cholinergic dysfunction, the symptomatic effects of ACh deficiency are treated.

10. The method of claim 9, wherein the long COVID disease includes inflammatory response, cholinergic dysfunction, or Alzheimer's-like symptoms.

11. A method of treating a disease in a subject in need thereof, comprising administering a therapeutically effective amount of the compound of claim 1 or the composition of claim 2 or claim 3 to the subject, wherein the disease is selected from inflammatory diseases, diseases associated with cholinergic dysfunction, autoimmune diseases, neurodegenerative disorders including Alzheimer's disease (AD), mild cognitive impairment (MCI), dementia with Lewy Bodies, subcortical vascular dementia, Parkinson's disease (PD), Huntington's disease (HD), and amyotrophic lateral sclerosis (ALS), or a combination thereof, and wherein by treating diseases associated with cholinergic dysfunction, the symptomatic effects of ACh deficiency are treated.

12. A method of treating a disease in a subject in need thereof, comprising administering a therapeutically effective amount of the compound of claim 1 or the composition of claim 2 or claim 3 to the subject, wherein the disease is selected from an inflammatory disease including, type 2 diabetes mellitus, hypertension, insulin resistance, hyperlipidemia, obesity, heart disease, metabolic syndrome, hyperthyroidism, nephrotic syndrome/kidney disease, proliferative diabetic retinopathy, age-related macular degeneration, chronic alcoholism, a viral infection, or a combination thereof.

13. The method of claim 12, wherein the inflammatory disease includes type 2 diabetes mellitus, metabolic syndrome, an ophthalmic disease, or a viral infection, or a combination thereof.

14. A method of treating a disease associated with cholinergic dysfunction, wherein by treating the disease associated with cholinergic dysfunction, the symptomatic effects of ACh deficiency are treated, comprising administering a therapeutically effective amount of the compound of claim 1 or the composition of claim 2 or claim 3 to the subject.

15. The method of claim 14, wherein the disease associated with cholinergic dysfunction is selected from Alzheimer's disease, mild cognitive impairment (MCI), dementia with Lewy Bodies, myasthenia gravis, multiple sclerosis (MS), autism spectrum disorder (ASD), Parkinson's disease, Huntington's disease, Lambert-Eaton myasthenic syndrome (LEMS), subcortical vascular dementia, opioid addiction, opiate use disorder, bipolar disorder, schizophrenia, metabolic syndrome, traumatic brain injury, or glaucoma, among other cholinergic disorders.

16. The method of claim 14, wherein the disease associated with cholinergic dysfunction includes dementia with Lewy Bodies, myasthenia gravis, multiple sclerosis (MS), autism spectrum disorder (ASD), opioid addiction, bipolar disorder, schizophrenia, an autoimmune disorder, or a combination thereof.

17. The method of any of claims 4-16, wherein the subject includes a genetic predisposition to increased risk of having AD or increased prevalence of beta-amyloid peptides or formation of beta-amyloid plaques.

18. The method of claim 17, wherein the genetic predisposition includes APOE e4 Gene, other Late-Onset Alzheimer's Genes (ABCA7, CLU, CR1, PICALM, PLD3, TREM2, or SORL1, or a combination thereof), Young-Onset Alzheimer's, Family History and Genetics Mutations, genetic mutations of amyloid precursor protein (APP), Presenilin 1 (PSEN1), Presenilin 2 (PSEN2), and Down Syndrome Mutations.

19. The method of claim 17, wherein the genetic predisposition includes APOE e4 gene, Young-Onset Alzheimer's, or Down Syndrome, or a combination thereof.

20. A method of modulating (e.g., inhibiting or reducing) expression or production of amyloid-β precursor protein (APP) in a subject in need thereof, comprising administering a therapeutically effective amount of the compound of claim 1 or the composition of claim 2 or claim 3 to the subject.

21. A method of modulating (e.g., inhibiting or reducing) expression or production of intracellular neurofibrillary tangles made of hyperphosphorylated tau protein in a subject in need thereof, comprising administering a therapeutically effective amount of the compound of claim 1 or the composition of claim 2 or claim 3 to the subject.

22. A method of modulating differentiation of a stem cell, comprising contacting the stem cell with an effective amount of the compound of claim 1 or the composition of claim 2 or claim 3.

23. A method of treating an esterase-related disease, e.g., a cholinesterase-related disease, e.g., an ACHE-related or BCHE-related disease, in a subject in need thereof, comprising administering to the subject a therapeutically effective amount of a compound, or a pharmaceutical composition comprising the compound, and a pharmaceutically acceptable carrier, wherein the compound is selected from:
A) Formula 1: 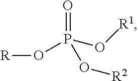
or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2: 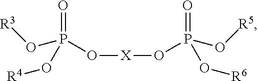
or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A: 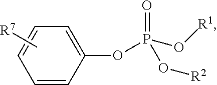
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B: 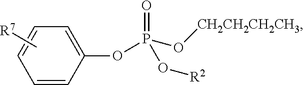
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C: 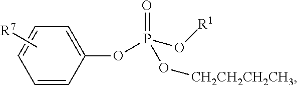
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D: 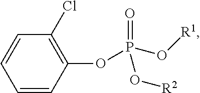
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A: 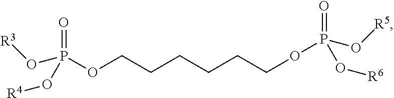
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B: 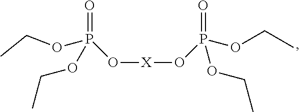
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C: 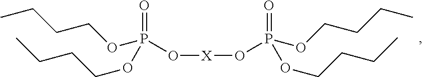
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D: 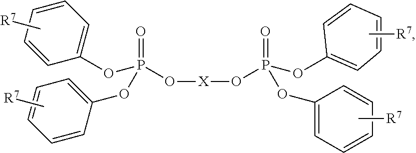
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3: 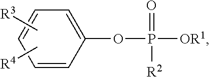
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
L) Formula 4: 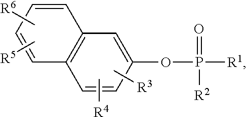
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5: 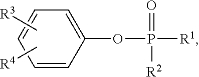
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or
R⁶ is H, C₁₋₆ alkyl, O—(C₁₋₆ alkyl), or an electron withdrawing group (e.g., halo, CN, or NO₂); or
N) Formula 6: 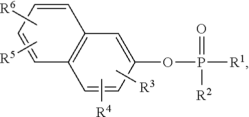
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7: 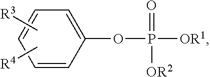
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8: 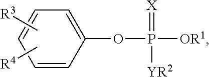
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or
R⁶ is H, C₁₋₆ alkyl, O—(C₁₋₆ alkyl), or an electron withdrawing group (e.g., halo, CN, or NO₂); or
Q) Formula 9: 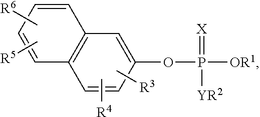
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂),
wherein the esterase-related disease includes:
a disease associate with the cholinergic anti-inflammatory pathway;
an autoimmune disease;
an inflammatory disease;
a disease associated with cholinergic dysfunction;
long COVID disease, or symptoms thereof, which may be associated with inflammation, cholinergic dysfunction, or formation of beta-amyloid plaques, or a combination thereof.

24. The method of claim 23, wherein the long COVID disease includes inflammatory response, cholinergic dysfunction, or Alzheimer's-like symptoms.

25. A method of treating a disease in a subject in need thereof, comprising administering a therapeutically effective amount of a compound, or a pharmaceutical composition comprising the compound, and a pharmaceutically acceptable carrier, wherein the compound is selected from:
A) Formula 1: 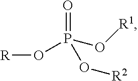
or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2: 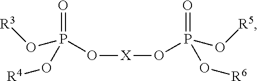
or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A: 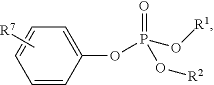
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B: 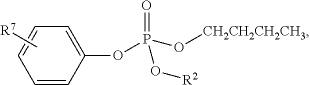
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C: 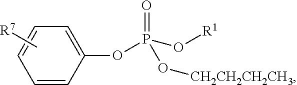
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D: 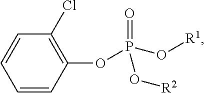
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A: 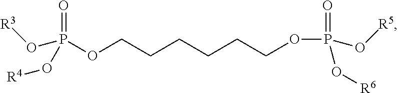
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B: 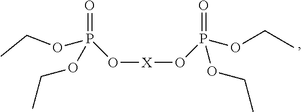
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C: 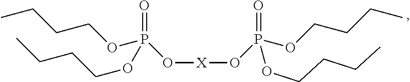
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D: 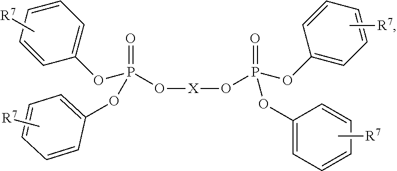
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3: 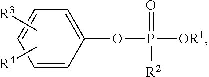
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
L) Formula 4: 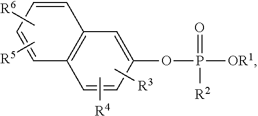
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5: 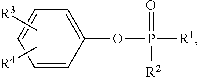
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
N) Formula 6: 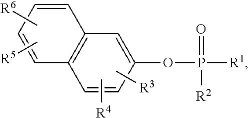
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7: 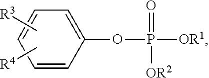
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or
R⁶ is H, C₁₋₆ alkyl, O—(C₁₋₆ alkyl), or an electron withdrawing group (e.g., halo, CN, or NO₂); or
P) Formula 8: 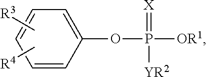
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
Q) Formula 9: 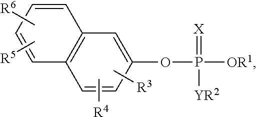
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂),
wherein the disease is selected from inflammatory diseases, diseases associated with cholinergic dysfunction, autoimmune diseases, neurodegenerative disorders including Parkinson's disease (PD), Huntington's disease (HD), and amyotrophic lateral sclerosis (ALS), or a combination thereof.

26. A method of treating a disease in a subject in need thereof, comprising administering a therapeutically effective amount of a compound, or a pharmaceutical composition comprising the compound, and a pharmaceutically acceptable carrier, wherein the compound is selected from:
A) Formula 1: 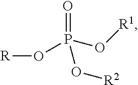
or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2: 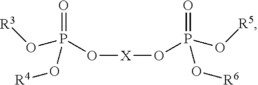
or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A: 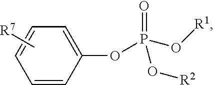
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B: 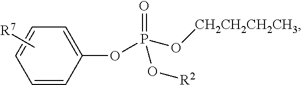
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C: 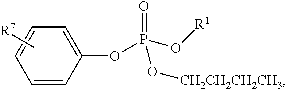
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D: 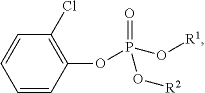
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A: 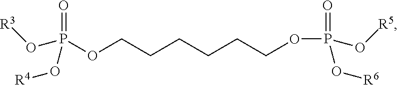
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B: 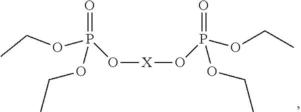
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C: 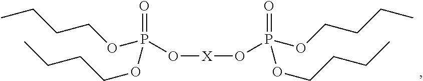
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D: 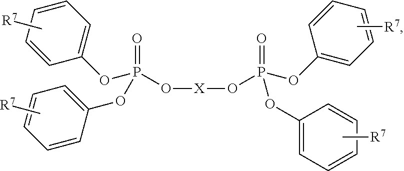
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3: 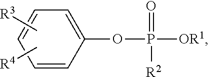
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
L) Formula 4: 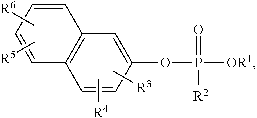
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5: 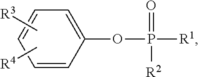
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or
R⁶ is H, C₁₋₆ alkyl, O—(C₁₋₆ alkyl), or an electron withdrawing group (e.g., halo, CN, or NO₂); or
N) Formula 6: 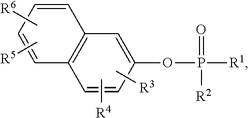
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7: 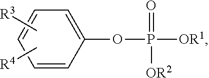
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8: 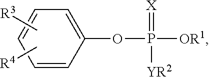
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or
R⁶ is H, C₁₋₆ alkyl, O—(C₁₋₆ alkyl), or an electron withdrawing group (e.g., halo, CN, or NO₂); or
Q) Formula 9: 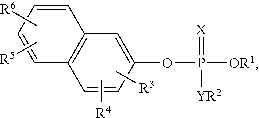
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂),
wherein the disease is selected from an inflammatory disease including, type 2 diabetes mellitus, hypertension, insulin resistance, hyperlipidemia, obesity, heart disease, metabolic syndrome, hyperthyroidism, nephrotic syndrome/kidney disease, proliferative diabetic retinopathy, age-related macular degeneration, chronic alcoholism, a viral infection, or a combination thereof.

27. The method of claim 26, wherein the inflammatory disease includes type 2 diabetes mellitus, metabolic syndrome, an ophthalmic disease, or a viral infection, or a combination thereof.

28. A method of treating a disease associated with cholinergic dysfunction in a subject in need thereof, comprising administering to the subject a therapeutically effective amount of a compound, or a pharmaceutical composition comprising the compound, and a pharmaceutically acceptable carrier, wherein the compound is selected from:
A) Formula 1: 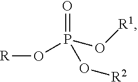
or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2: 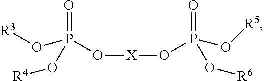
or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A: 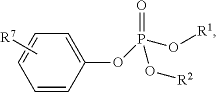
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B: 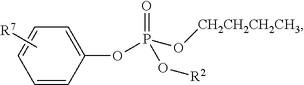
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C: 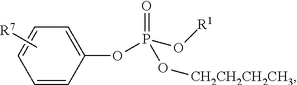
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D: 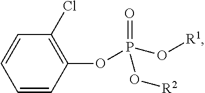
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A: 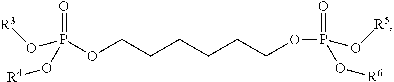
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B: 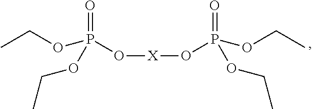
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C: 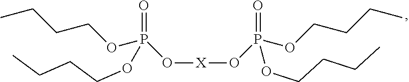
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D: 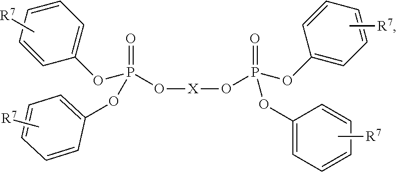
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3: 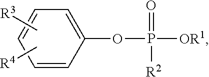
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
L) Formula 4: 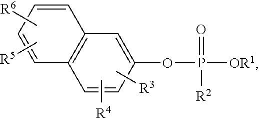
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5: 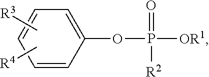
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
N) Formula 6: 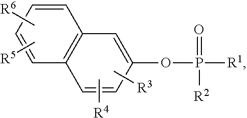
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7: 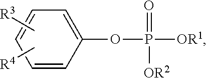
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8: 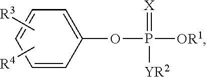
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
Q) Formula 9: 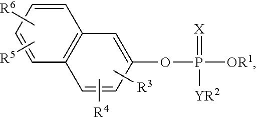
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂).

29. The method of claim 28, wherein the disease associated with cholinergic dysfunction is selected from dementia with Lewy Bodies, myasthenia gravis, multiple sclerosis (MS), autism spectrum disorder (ASD), Parkinson's disease, Huntington's disease, Lambert-Eaton myasthenic syndrome (LEMS), subcortical vascular dementia, opioid addiction, opiate use disorder, bipolar disorder, schizophrenia, metabolic syndrome, or glaucoma, among other cholinergic disorders.

30. The method of claim 28, wherein the disease associated with cholinergic dysfunction includes dementia with Lewy Bodies, myasthenia gravis, multiple sclerosis (MS), autism spectrum disorder (ASD), opioid addiction, bipolar disorder, schizophrenia, an autoimmune disorder, or a combination thereof.

31. The method of any of claims 23-30, wherein the subject includes a genetic predisposition to increased risk of having AD or increased prevalence of beta-amyloid peptides or formation of beta-amyloid plaques.

32. The method of claim 31, wherein the genetic predisposition includes APOE e4 Gene, other Late-Onset Alzheimer's Genes (ABCA7, CLU, CR1, PICALM, PLD3, TREM2, or SORL1, or a combination thereof), Young-Onset Alzheimer's, Family History and Genetics Mutations, genetic mutations of amyloid precursor protein (APP), Presenilin 1 (PSEN1), Presenilin 2 (PSEN2), and Down Syndrome Mutations.

33. The method of claim 31, wherein the genetic predisposition includes APOE e4 gene, Young-Onset Alzheimer's, or Down Syndrome, or a combination thereof.

34. A method of modulating (e.g., inhibiting or reducing) expression or production of amyloid-β precursor protein (APP) in a subject in need thereof, comprising administering to the subject a therapeutically effective amount of a compound, or a pharmaceutical composition comprising the compound, and a pharmaceutically acceptable carrier, wherein the compound is selected from:
A) Formula 1: 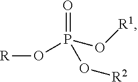
or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2: 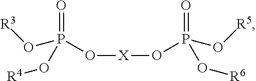
or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A: 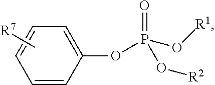
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B: 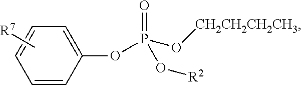
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C: 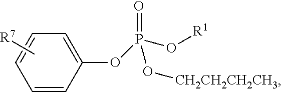
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D: 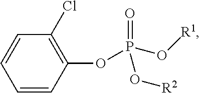
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A: 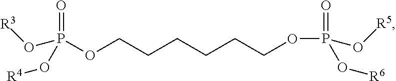
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B: 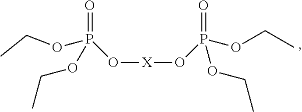
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C: 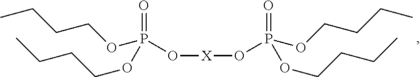
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D: 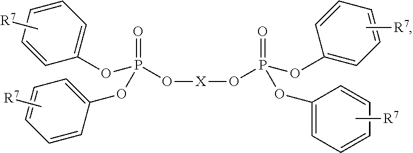
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3: 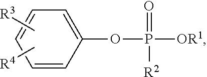
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
L) Formula 4: 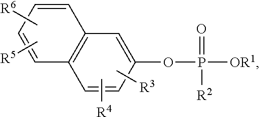
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5: 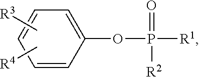
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
N) Formula 6: 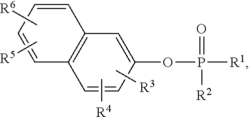
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7: 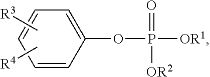
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8: 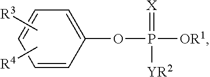
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
Q) Formula 9: 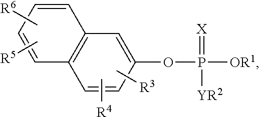
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂).

35. A method of modulating (e.g., inhibiting or reducing) expression or production of intracellular neurofibrillary tangles made of hyperphosphorylated tau protein in a subject in need thereof, comprising administering to the subject a therapeutically effective amount of a compound, or a pharmaceutical composition comprising the compound, and a pharmaceutically acceptable carrier, wherein the compound is selected from:
A) Formula 1: 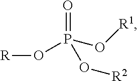
or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2: 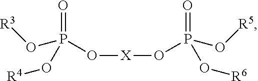
or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A: 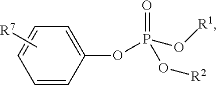
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B: 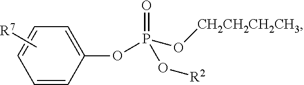
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C: 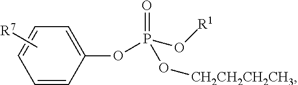
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D: 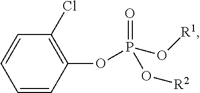
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A: 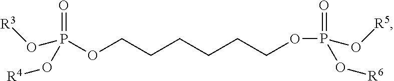
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B: 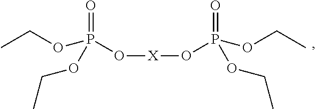
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C: 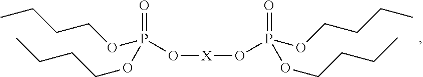
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D: 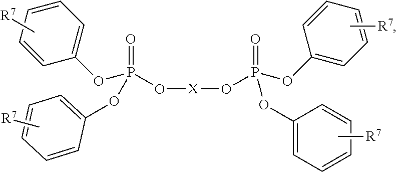
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3: 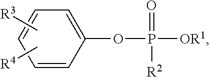
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
L) Formula 4: 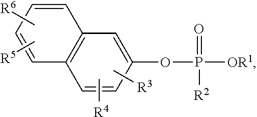
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5: 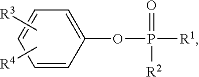
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
N) Formula 6: 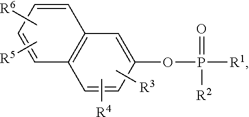
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or
R⁶ is H, C₁₋₆ alkyl, O—(C₁₋₆ alkyl), or an electron withdrawing group (e.g., halo, CN, or NO₂); or
O) Formula 7: 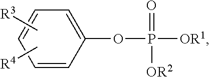
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8: 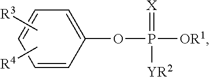
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
Q) Formula 9: 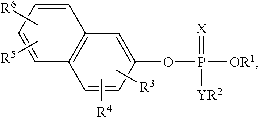
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂).

36. A method of modulating differentiation of a stem cell, comprising contacting the stem cell with an effective amount of a compound, or a pharmaceutical composition comprising the compound, and a pharmaceutically acceptable carrier, wherein the compound is selected from:
A) Formula 1: 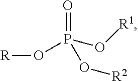
or a pharmaceutically acceptable salt thereof,
wherein R is C₅₋₁₀ aryl, or C₅₋₆ heteroaryl containing 1-4 heteroatoms selected, independently, from N, O, or S, wherein the C₅₋₁₀ aryl, or C₅₋₆ heteroaryl can be optionally substituted with 1 to 5 of, independently, R⁷;
R¹ and R² are, independently, C₁₋₁₂ alkyl, and the C₁₋₁₂ alkyl can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
B) Formula 2: 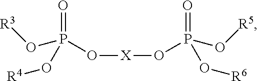
or a pharmaceutically acceptable salt thereof,
wherein R³, R⁴, R⁵ and R⁶ are independently C₅₋₁₀ aryl, C₅₋₆ heteroaryl containing 1-4 heteroatoms selected, independently, from N, O, or S, or C₁₋₁₂ alkyl, wherein the C₅₋₁₀ aryl, C₅₋₆ heteroaryl, and C₁₋₁₂ alkyl can be optionally substituted with R⁷;
X is C₁₋₁₂ alkyl which can be optionally substituted with R⁷;
each R⁷ is, independently, halogen, cyano, C₁₋₆ alkyl, C₂₋₆ alkenyl, C₂₋₆ alkynyl, —OR^{a}, —C(O)R^{a}, —OC(O)R^{a}, —C(O)OR^{a}, —C(O)NR^{a}R^{b}, —NR^{a}R^{b}, —NR^{a}S(O)₂R^{b}, —S(O)₁₋₂R^{a}, or —S(O)₂NR^{a}R^{b}; and
R^{a} and R^{b} are, independently, H or C₁₋₆ alkyl; or
C) Formula 1A: 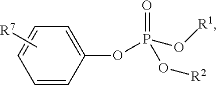
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
D) Formula 1B: 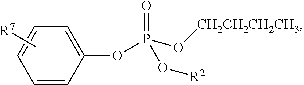
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
E) Formula 1C: 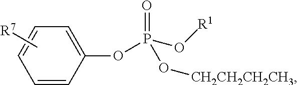
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
F) Formula 1D: 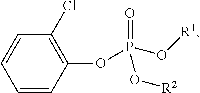
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
G) Formula 2A: 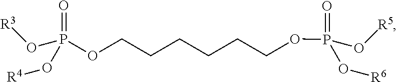
or a pharmaceutically acceptable salt thereof,
wherein, R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
H) Formula 2B: 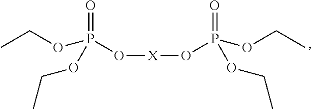
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
I) Formula 2C: 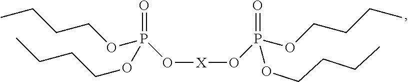
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
J) Formula 2D: 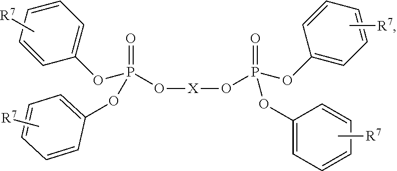
or a pharmaceutically acceptable salt thereof,
wherein,
R¹ is optionally substituted C₁₋₁₂ alkyl;
R² is optionally substituted C₁₋₁₂ alkyl;
each R³, R⁴, R⁵ and R⁶ are, independently, C₅₋₁₀ aryl, C₅₋₆ heteroaryl containing 1-4 heteroatoms selected from N, O and S, or C₁₋₁₂ alkyl, wherein the C₅₋₁₀ aryl, C₅₋₆ heteroaryl, and C₁₋₁₂ alkyl can be optionally substituted with R⁷;
R⁷ is halogen, cyano, C₁₋₆ alkyl, C₂₋₆ alkenyl, C₂₋₆ alkynyl, OR^{a}, C(O)R^{a}, C(O)OR^{a}, C(O)NR^{a}R^{b}, NR^{a}R^{b}, NR^{a}S(O)₂R^{b}, S(O)₁₋₂R^{a}, or S(O)₂NR^{a}R^{b}, wherein R^{a} and R^{b} are independently H or C₁₋₆ alkyl; and
X is C₁₋₁₂ alkyl which can be optionally substituted with R⁷; or
K) Formula 3: 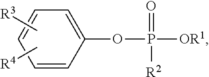
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
L) Formula 4: 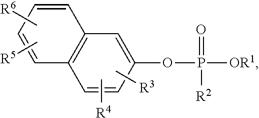
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
M) Formula 5: 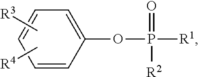
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
N) Formula 6: 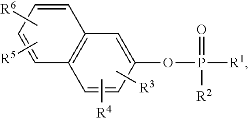
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
O) Formula 7: 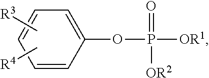
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
P) Formula 8: 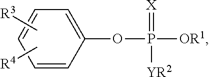
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or NO₂);
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂); or
Q) Formula 9: 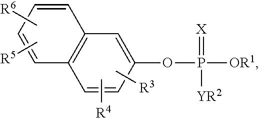
or a pharmaceutically acceptable salt thereof,
wherein
X is O or S;
Y is O or N;
R¹ is H, C₁₋₆ alkyl, C₁₋₆ alkenyl, or C₆₋₁₂ aryl;
R² is H, C₁₋₆ alkyl, C₁₋₆ alkenyl, or C₆₋₁₂ aryl;
R³ is H, C₁₋₆ alkyl, O—(C₁₋₆ alkyl), or an electron withdrawing group (e.g., halo, CN, or
R⁴ is H, C₁₋₆ alkyl, O—(C₁₋₆ alkyl), or an electron withdrawing group (e.g., halo, CN, or NO₂);
R⁵ is H, C₁₋₆ alkyl, O—(C₁₋₆ alkyl), or an electron withdrawing group (e.g., halo, CN, or NO₂); and
R⁶ is H, C₁₋₆ alkyl, O—(C₁₋₆ alkyl), or an electron withdrawing group (e.g., halo, CN, or NO₂).

37. The method of one of claims 23-36, wherein the compound is:
2-Bromophenyl di-n-butyl phosphate;
3-Bromophenyl n-dibutyl phosphate;
Dibutyl 3-chlorophenyl phosphate;
4-Bromophenyl di-n-butyl phosphate;
2-Chlorophenyl dimethyl phosphate;
2-Chlorophenyl diethyl phosphate;
2-Chlorophenyl di-n-propyl phosphate;
2-Chlorophenyl di-i-propyl phosphate;
Di-n-butyl 2-chlorophenyl phosphate;
Di-i-butyl 2-Chlorophenyl phosphate;
2-Chlorophenyl di-n-pentyl phosphate;
2-Chlorophenyl dicyclohexyl phosphate;
Di-n-butyl 4-chlorophenyl phosphate;
Di-n-butyl 2,4-dichlorophenyl phosphate;
Di-n-butyl 2-fluorophenyl phosphate;
Di-n-butyl 3-fluorophenyl phosphate;
Dibutyl 2-chlorophenyl thiophosphate;
n-Butyl bis(2,4-dichlorophenyl) phosphate;
Butyl bis(2-chlorophenyl) phosphate;
2-Chlorophenyl diphenyl phosphate;
Di-n-butyl 4-nitrophenyl phosphate;
Di-n-butyl 3-nitrophenyl phosphate;
Di-n-butyl 2-methylphenyl phosphate;
Di-n-butyl 3-methylphenyl phosphate;
Di-n-butyl 4-methylphenyl phosphate;
Di-n-butyl 3,4-dimethylphenyl phosphate;
Di-n-butyl 3,5-dimethylphenyl phosphate;
Diethyl 3,5-dimethylphenyl phosphate;
Di-n-butyl 3,4,5-trimethylphenyl phosphate;
Benzyl di-n-butyl phosphate;
Di-n-butyl 2-methylbenzyl phosphate;
Di-n-butyl 3-methylbenzyl phosphate;
Di-n-butyl 4-methylbenzyl phosphate;
Di-n-butyl 1-napthyl phosphate;
Di-n-butyl 2-napthyl phosphate;
Diethyl 2-napthyl phosphate;
Di-n-butyl 2-phenylethyl phosphate;
Butyl 2-chlorophenyl hexylphosphonate;
Bis(2-chlorophenyl) ethylphosphonate;
Butyl 2-chlorophenyl ethylphosphonate;
Bis(3-nitrophenyl) ethylphosphonate;
Butyl p-nitrophenyl ethylphosphonate;
Bis (p-nitrophenyl) ethylphosphonate;
Bis(2-chlorophenyl) hexylphosphonate;
Bis(2-chlorophenyl) n-butylphosphonate;
Bis(2-chlorophenyl) ethylphosphonate;
2-Chlorophenyl di-n-pentylphosphinate;
2-Chlorophenyl di-n-butylphosphinate;
Phenyl di-n-pentylphosphinate;
4-nitrophenyl dipentylphosphinate;
O-n-butyl-O-2-chlorophenyl-N-hexyl phosphoramidate;
Bis(2-chlorophenyl) N-hexyl phosphoramidate;
Tetraethyl propane-1,3-diyl bisphosphate;
Tetraethyl butane-1,4-diyl bisphosphate;
Tetraethyl pentane-1,5-diyl bisphosphate;
Tetraethyl hexane-1,6-diyl bisphosphate;
Tetraethyl heptane-1,7-diyl bisphosphate;
Tetraethyl 3-oxapentane-1,5-diyl bisphosphate;
Tetrabutyl propane-1,3-diyl bisphosphate;
Tetrabutyl butane-1,4-diyl bisphosphate;
Tetrabutyl pentane-1,5-diyl bisphosphate;
Tetrabutyl hexane-1,6-diyl bisphosphate;
Tetrabutyl heptane-1,7-diyl bisphosphate;
Tetrabutyl octane-1,8-diyl bisphosphate;
Tetrabutyl 3-oxapentane-1,5-diyl bisphosphate;
Tetrabutyl 3-hexene-1,6-diyl bisphosphate;
Tetraphenyl hexane-1,6-diyl bisphosphate;
O,O,O′,O′-tetrabutyl S,S′-(ethane-1,2-diyl) Bis(phosphorothioate);
O,O,O′,O′-tetrabutyl S,S′-(propane-1,3-diyl) Bis(phosphorothioate);
O,O,O′,O′-tetrabutyl S,S′-(butane-1,4-diyl) Bis(phosphorothioate);
O,O,O′,O′-tetrabutyl S,S′-(pentane-1,5-diyl) Bis(phosphorothioate);
O,O,O′,O′-tetrabutyl S,S′-(hexane-1,6-diyl) Bis(phosphorothioate);
O,O,O′,O′-tetrabutyl S,S′-(heptane-1,7-diyl) Bis(phosphorothioate);
O,O,O′,O′-tetrabutyl S,S′-(octane-1,8-diyl) Bis(phosphorothioate);
O,O,O′,O′-tetrabutyl S,S′-(nonane-1,9-diyl) Bis(phosphorothioate);
O,O,O′,O′-tetrabutyl S,S′-(decane-1,10-diyl) Bis(phosphorothioate);
S′,S′-(hexane-1,6-diylbis(azenediyl)) Bis(2-oxoethane-2,1-diyl) O,O,O′,O′-Tetraethyl Diphosphorodithioate;
S′,S′-(butane-1,4-diylbis(azenediyl)) Bis(2-oxoethane-2,1-diyl) O,O,O′,O′-Tetrabutyl Diphosphorodithioate;
S′,S′-(pentane-1,5-diylbis(azenediyl)) Bis(2-oxoethane-2,1-diyl) O,O,O′,O′-Tetrabutyl Diphosphorodithioate;
S′,S′-(hexane-1,6-diylbis(azenediyl)) Bis(2-oxoethane-2,1-diyl) O,O,O′,O′-Tetrabutyl Diphosphorodithioate;
S′,S′-(heptane-1,7-diylbis(azenediyl)) bis(2-oxoethane-2,1-diyl) O,O,O′,O′-Tetrabutyl Diphosphorodithioate;
S′,S′-(octane-1,8-diylbis(azenediyl)) Bis(2-oxoethane-2,1-diyl) O,O,O′,O′-Tetrabutyl Diphosphorodithioate;
S′,S′-(nonane-1,9-diylbis(azenediyl)) Bis(2-oxoethane-2,1-diyl) O,O,O′,O′-Tetrabutyl Diphosphorodithioate;
S′,S′-(decane-1,10-diylbis(azenediyl)) Bis(2-oxoethane-2,1-diyl) O,O,O′,O′-Tetrabutyl Diphosphorodithioate;
Di-n-butyl 4-methylbenzylphosphonate;
Di-n-butyl 4-fluorobenzylphosphonate;
Di-n-butyl 4-methoxybenzylphosphonate;
Di-n-butyl 4-chlorobenzylphosphonate;
Di-n-butyl 1-naphthylmethylphosphonate;
Di-n-butyl benzylphosphonate;
Di-n-butyl 3,5-dimethylbenzylphosphonate;
Dimethyl phenyl phosphate;
Diethyl phenyl phosphate;
Di-n-propyl phenyl phosphate;
Di-n-butyl phenyl phosphate;
Di-n-pentyl phenyl phosphate;
Di-cyclohexyl phenyl phosphate; or
(±)-n-Butyl ethyl phenyl phosphate;
or a pharmaceutically acceptable salt thereof.

38. A compound described herein for use as described herein.
